(19) United States
(12) Patent Application Publication
(10) Pub. No.: US 2024/0199597 A1
(43) Pub. Date: Jun. 20, 2024

(54) SMALL MOLECULES FOR THE TREATMENT OF KINASE-RELATED DISEASES

(71) Applicant: Athos Therapeutics, Inc., Torrance, CA (US)

(72) Inventors: Dimitrios Iliopoulos, Los Angeles, CA (US); David G. Ho, Monterey Park, CA (US); Iordanis Karagiannidis, North Hollywood, CA (US); Phithi Nguyen, Anaheim, CA (US); Dimitra Chalkia, Fullerton, CA (US)

(21) Appl. No.: 18/551,281

(22) PCT Filed: Mar. 22, 2022
(86) PCT No.: PCT/US2022/071268

§ 371 Date: Sep. 19, 2023

Related U.S. Application Data
(60) Provisional application No. 63/165,604, filed on Mar. 24, 2021.

(51) Int. Cl. C07D 417/14; A61K 31/517; A61K 31/5377; A61K 31/541; A61P 35/00; C07D 401/14; C07D 403/14; C07D 405/14; C07D 409/14; C07D 413/14

(52) U.S. Cl. CPC C07D 417/14; A61K 31/517; A61K 31/5377; A61K 31/541; A61P 35/00; C07D 401/14; C07D 403/14; C07D 405/14; C07D 409/14; C07D 413/14

(57) ABSTRACT

Disclosed herein are quinazolinyl compounds, compositions, and methods of use thereof. The compounds may be used in the treatment of kinase-related disorders (including cancer, autoimmune disease, and Duchenne muscular dystrophy).

### CROSS REFERENCE TO RELATED APPLICATIONS

The present application claims the benefit of priority to U.S. Provisional Patent Application No. 63/165,604, filed Mar. 24, 2021, the entirety of which is hereby incorporated by reference herein.

### Field

The present application relates to the fields of chemistry and medicine. More specifically, the application relates to compounds that are useful in the treatment of kinase-related disorders (including cancer, autoimmune disease, and Duchenne muscular dystrophy).

### BACKGROUND

### Description of the Related Technology

A protein kinase selectively modifies other proteins by covalently adding phosphates to them (phosphorylation). Phosphorylation usually results in a functional change of the target protein (substrate) by changing enzyme activity, cellular location, or association with other proteins. A protein kinase inhibitor is a type of enzyme inhibitor that blocks the action of one or more protein kinases. Phosphorylation regulates many biological processes, and protein kinase inhibitors can be used to treat diseases due to hyperactive protein kinases (including mutant or overexpressed kinases in cancer, autoimmune disease, and Duchenne muscular dystrophy) or to modulate cell functions to overcome other disease drivers.

### SUMMARY

Several embodiments disclosed herein pertain to quinazolinyl compounds, methods of using quinazolinyl compounds, compositions comprising quinazolinyl compounds, and methods of treatment using quinazolinyl compounds. Quinazolinyl compounds may be represented by the following structure and numbering convention:

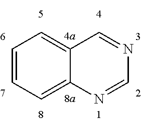

In several embodiments, the quinazolinyl compound is substituted at the 2-position, the 4-position, the 6-position, the 7-position, or combinations of any one of the foregoing. In several embodiments, the quinazoline compound comprises an amine substituent bonded to the quinazoline ring at the 4-position. In several embodiments, the amine at the 4-position is bonded to a heteroaryl or heterocyclic substituent. In several embodiments, the quinazoline compound comprises a methoxy group at the 6-position. In several embodiments, the quinazoline compound comprises an alkoxy or alkynyl group at the 7-position. In several embodiments, the alkoxy or alkynyl group at the 7-position comprises a pendant heterocyclic group.

As disclosed elsewhere herein, in several embodiments, the quinazolinyl compound is substituted at the 2-position. In several embodiments, the substituent at the 2-position may include an optionally substituted 6-10 membered aryl, optionally substituted 3-10 membered heterocyclyl, optionally substituted 5-10 membered heteroaryl, optionally substituted carbamide, —CN, amino, monosubstituted amino, a disubstituted amino, or combinations of the foregoing.

In several embodiments, the 2-position substituent may be optionally substituted as disclosed herein. In several embodiments, when the 2-position substituent comprises one or more optional substituents, the one or more optional substitutions may be independently selected from the group consisting of C₁-C₃ alkyl, halo, cyano, hydroxy, and C₁-C₃ alkoxy. In several embodiments, when the 2-position substituent comprises one or more optional substituents, the one or more optional substitutions may be independently selected from the group consisting amino, —OH, optionally substituted C₁-C₆ alkyl, and halogen.

As disclosed elsewhere herein, in several embodiments, the quinazolinyl compound is substituted at the 4-position with an amine. In several embodiments, the quinazoline compound comprises the amine at the 4-position comprising a cyclic substituent (either pendant or directly bonded to the amine) or the amine is part of a cyclic substituent. In several embodiments, the substituent at the 4-position may be an optionally substituted heterocyclyl.

In several embodiments, the 4-position substituent may be optionally substituted as disclosed herein. In several embodiments, when the 4-position substituent comprises one or more optional substituents, the one or more optional substitutions may be independently selected from the group consisting of C₁-C₃ alkyl, C₁-C₃ alkenyl, halo, cyano, hydroxy, and C₁-C₃ alkoxy. In several embodiments, when the 4-position substituent comprises one or more optional substituents, the one or more optional substitutions may be independently selected from the group consisting amino, —OH, optionally substituted C₁-C₆ alkyl, and halogen.

As disclosed elsewhere herein, in several embodiments, the quinazolinyl compound is substituted at the 7-position with alkoxy or (heterocyclyl)alkynyl. In several embodiments, the alkoxy comprises an optionally substituted 2-10 membered heteroalkyl.

In several embodiments, the 7-position may be optionally substituted as described herein. In several embodiments, when the 7-position substituent comprises one or more optional substituents, the one or more optional substitutions may be independently selected from the group consisting of C₁-C₃ alkyl, halo, cyano, hydroxy, and C₁-C₃ alkoxy. In several embodiments, when the 7-position substituent comprises one or more optional substituents, the one or more optional substitutions may be independently selected from the group consisting amino, —OH, optionally substituted C₁-C₆ alkyl, and halogen.

Several embodiments disclosed herein pertain to quinazolinyl compounds of Formula (I), methods of using those quinazolinyl compounds, compositions comprising thos quinazolinyl compounds, and methods of treatment using those quinazolinyl compounds:

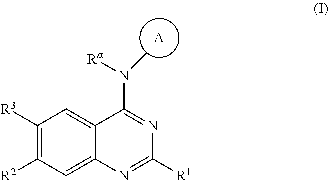

or a stereoisomer, tautomer, or pharmaceutically acceptable salt thereof. In several embodiments, R′ is selected from the group consisting of optionally substituted 6-10 membered aryl, optionally substituted 3-10 membered heterocyclyl, optionally substituted 5-10 membered heteroaryl, optionally substituted carbamide, —CN, and —NR⁴R⁵; each of R⁴ and R⁵ is independently selected from hydrogen, optionally substituted C₁-C₆ alkyl, or optionally substituted C₃-C₆ carbocyclyl; or alternatively, R⁴ and R⁵ taken together form an optionally substituted 3-10 membered heterocyclyl; R² is-OR⁶ or optionally substituted (heterocyclyl)alkynyl; R⁶ is selected from the group consisting of methyl, optionally substituted 2-10 membered heteroalkyl, (carbocyclyl)alkyl, and (heterocyclyl)alkyl; R³ is selected from the group consisting of hydrogen, halogen, and C₁₋₆ alkoxy; R^{a} is hydrogen or optionally substituted C₁-C₁₀ alkyl; and the A ring is an optionally substituted heteroaryl or an optionally substituted heterocyclyl.

In several embodiments, the compound of Formula (I) is represented by one or more of the following structures:

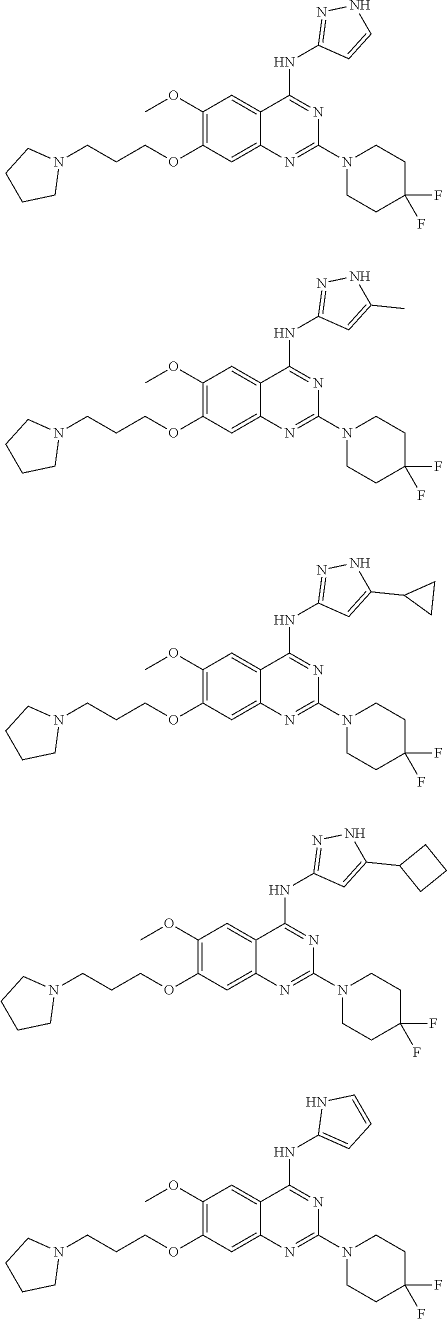 [SMILES: COc1cc2c(Nc3cc[nH]n3)nc(N3CCC(F)(F)CC3)nc2cc1OCCCN1CCCC1 ; COc1cc2c(Nc3cc(C)[nH]n3)nc(N3CCC(F)(F)CC3)nc2cc1OCCCN1CCCC1 ; COc1cc2c(Nc3cc(C4CC4)[nH]n3)nc(N3CCC(F)(F)CC3)nc2cc1OCCCN1CCCC1 ; COc1cc2c(Nc3cc(C4CCC4)[nH]n3)nc(N3CCC(F)(F)CC3)nc2cc1OCCCN1CCCC1 ; COc1cc2c(Nc3ccc[nH]3)nc(N3CCC(F)(F)CC3)nc2cc1OCCCN1CCCC1]

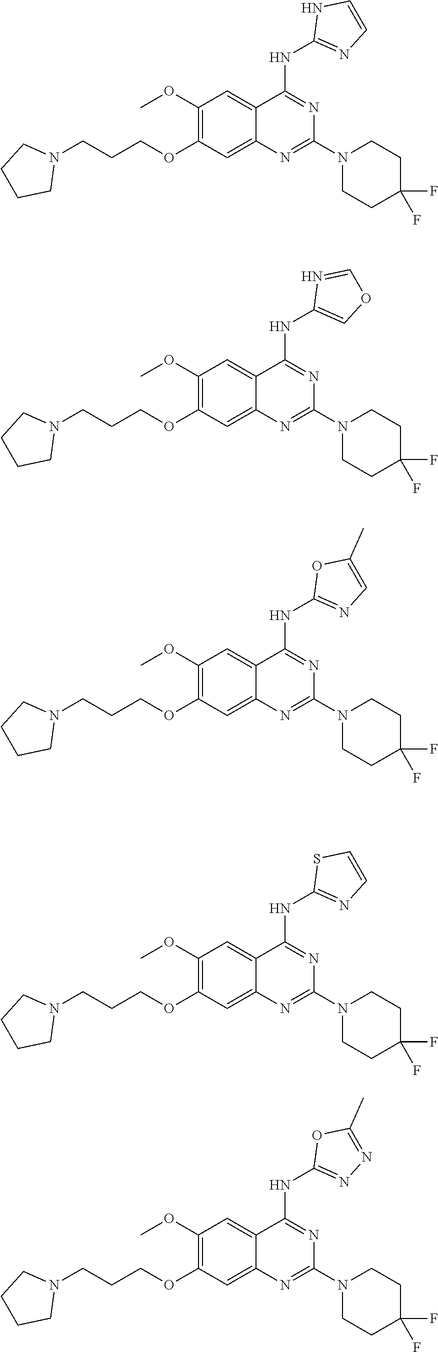

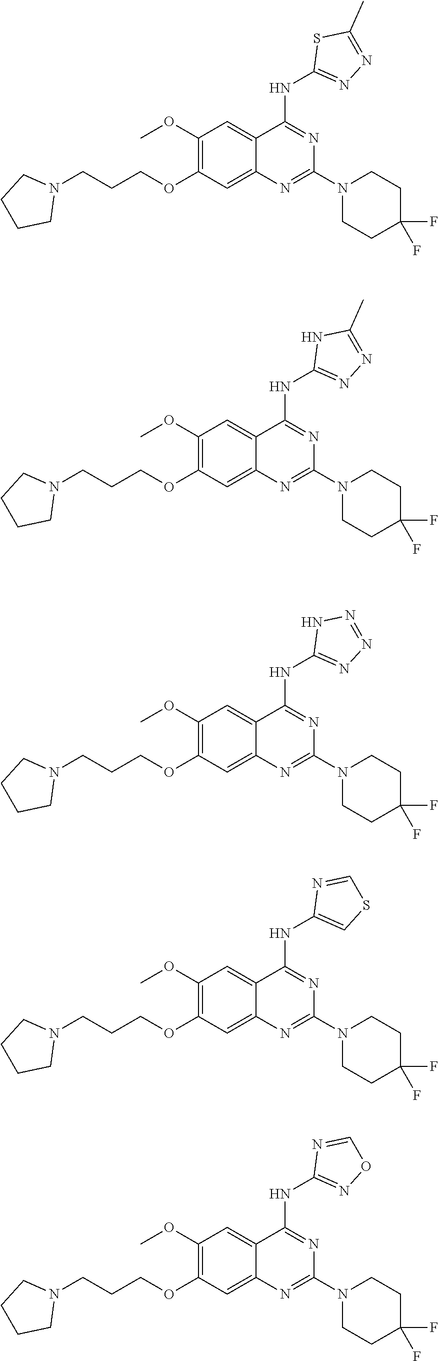 [SMILES: COc1cc2c(Nc3nnc(C)s3)nc(N3CCC(F)(F)CC3)nc2cc1OCCCN1CCCC1 ; COc1cc2c(Nc3nnc(C)[nH]3)nc(N3CCC(F)(F)CC3)nc2cc1OCCCN1CCCC1 ; COc1cc2c(Nc3nnn[nH]3)nc(N3CCC(F)(F)CC3)nc2cc1OCCCN1CCCC1 ; COc1cc2c(Nc3cscn3)nc(N3CCC(F)(F)CC3)nc2cc1OCCCN1CCCC1 ; COc1cc2c(Nc3ncon3)nc(N3CCC(F)(F)CC3)nc2cc1OCCCN1CCCC1]

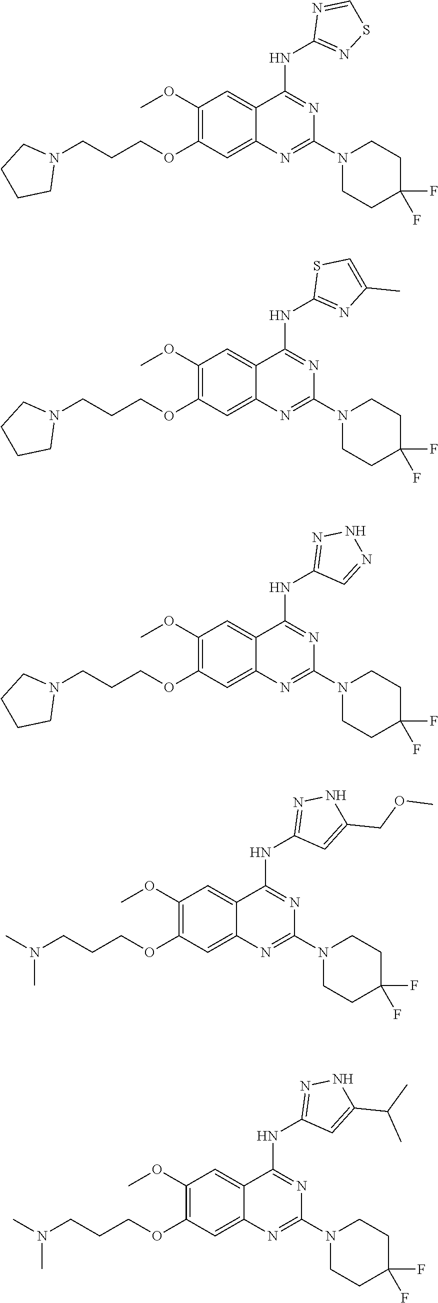

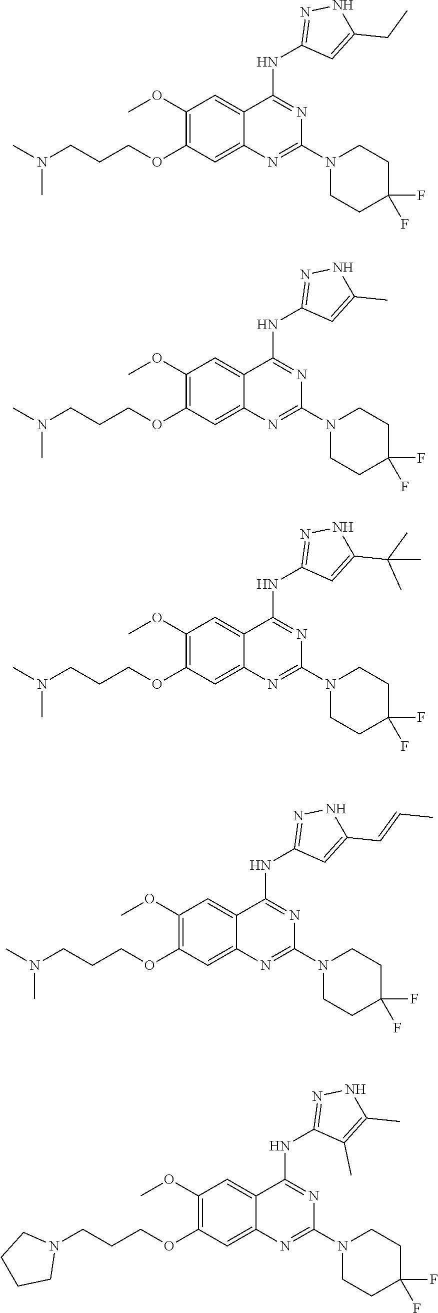

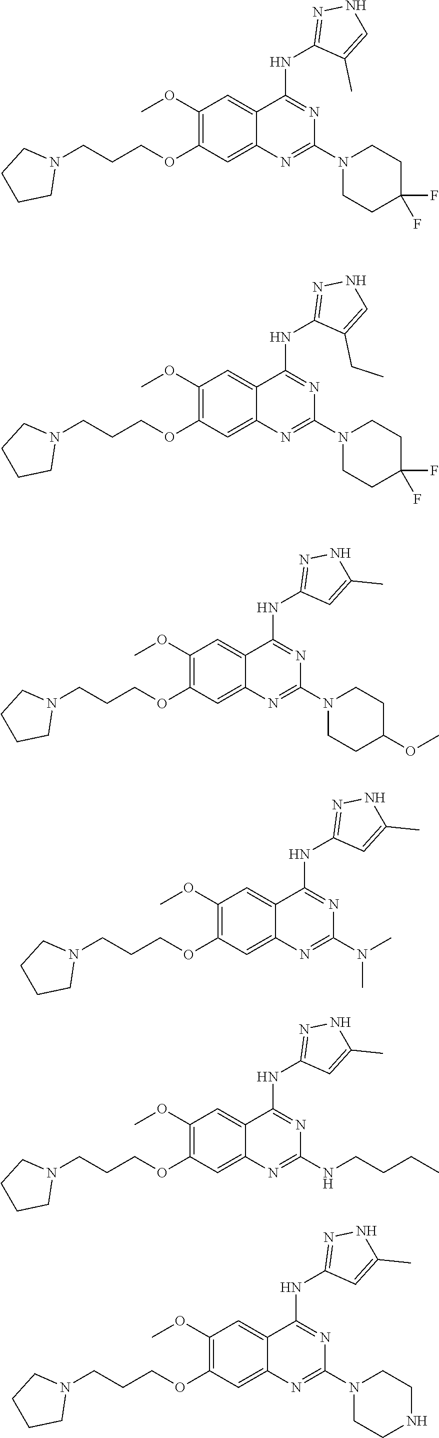

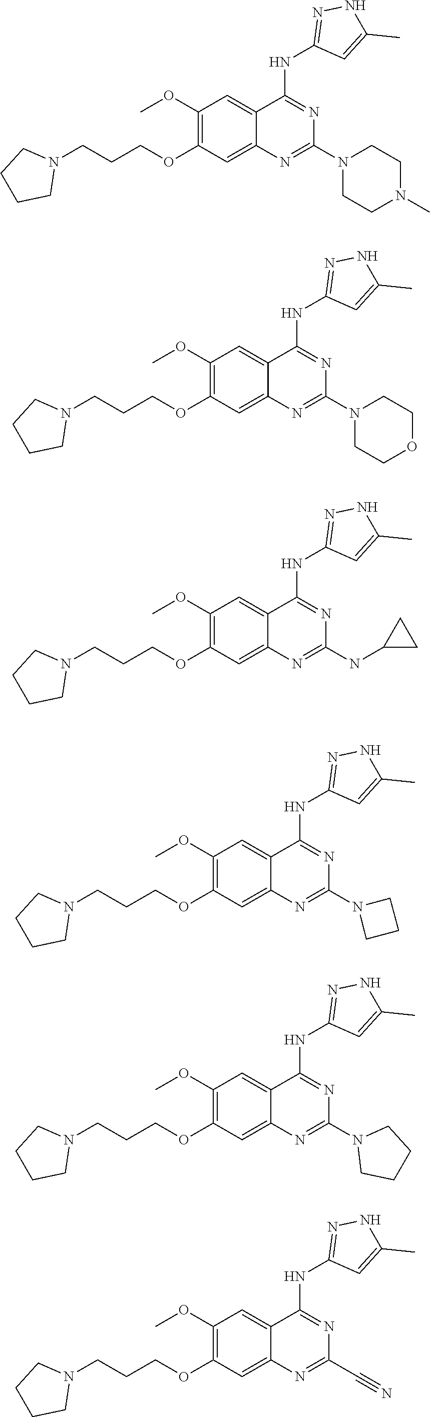

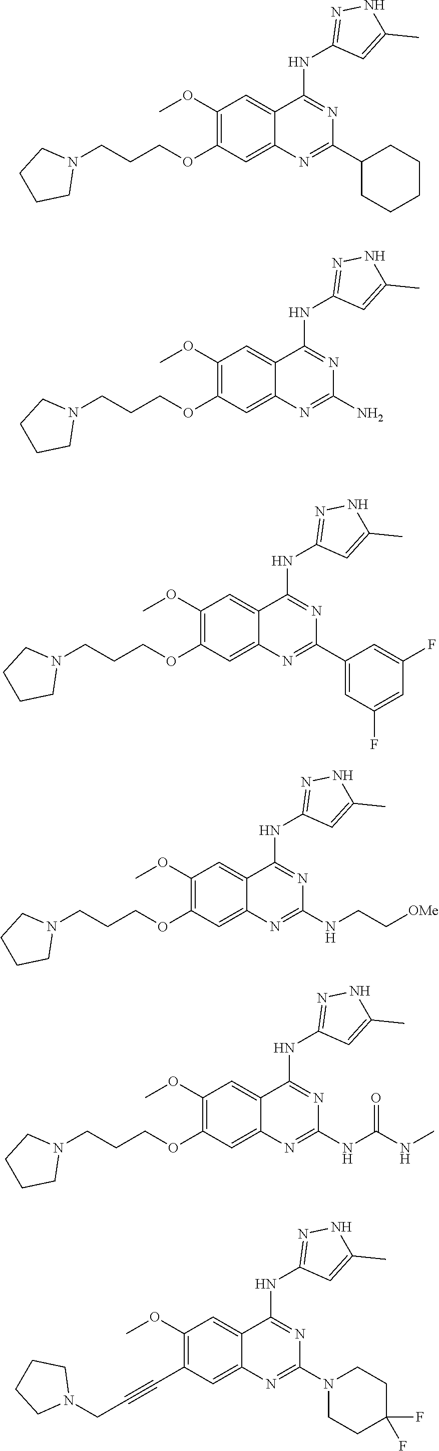 [SMILES: COc1cc2c(Nc3cc(C)[nH]n3)nc(C3CCCCC3)nc2cc1OCCCN1CCCC1 ; COc1cc2c(Nc3cc(C)[nH]n3)nc(N)nc2cc1OCCCN1CCCC1 ; COc1cc2c(Nc3cc(C)[nH]n3)nc(-c3cc(F)cc(F)c3)nc2cc1OCCCN1CCCC1 ; COCCNc1nc(Nc2cc(C)[nH]n2)c2cc(OC)c(OCCCN3CCCC3)cc2n1 ; CNC(=O)Nc1nc(Nc2cc(C)[nH]n2)c2cc(OC)c(OCCCN3CCCC3)cc2n1 ; COc1cc2c(Nc3cc(C)[nH]n3)nc(N3CCC(F)(F)CC3)nc2cc1C#CCN1CCCC1]

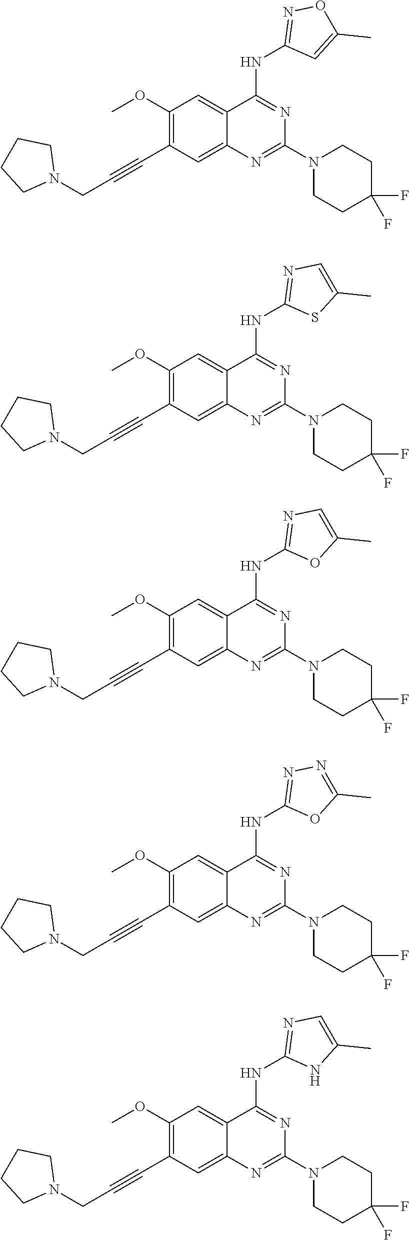

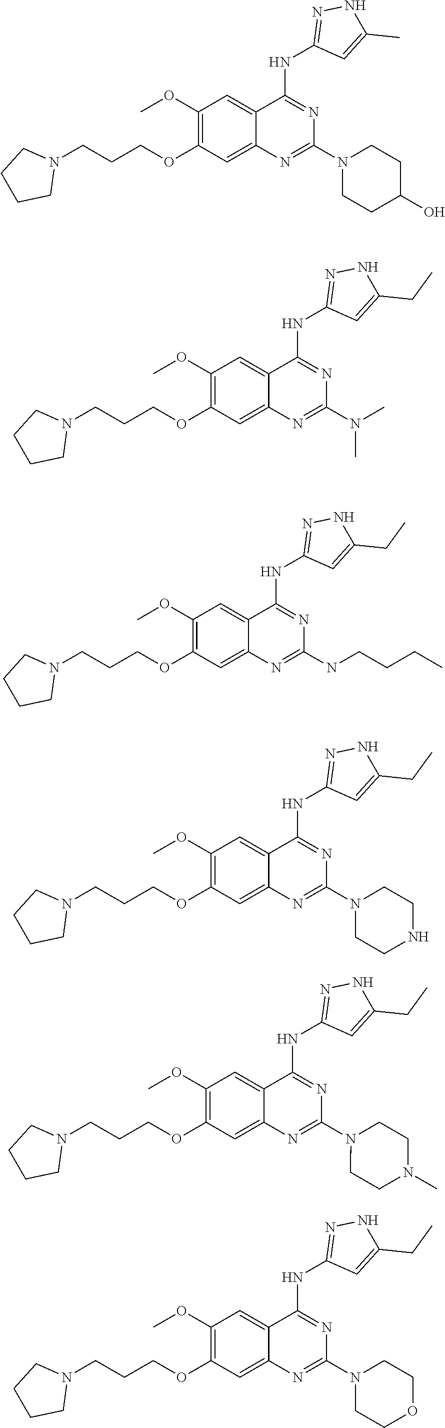

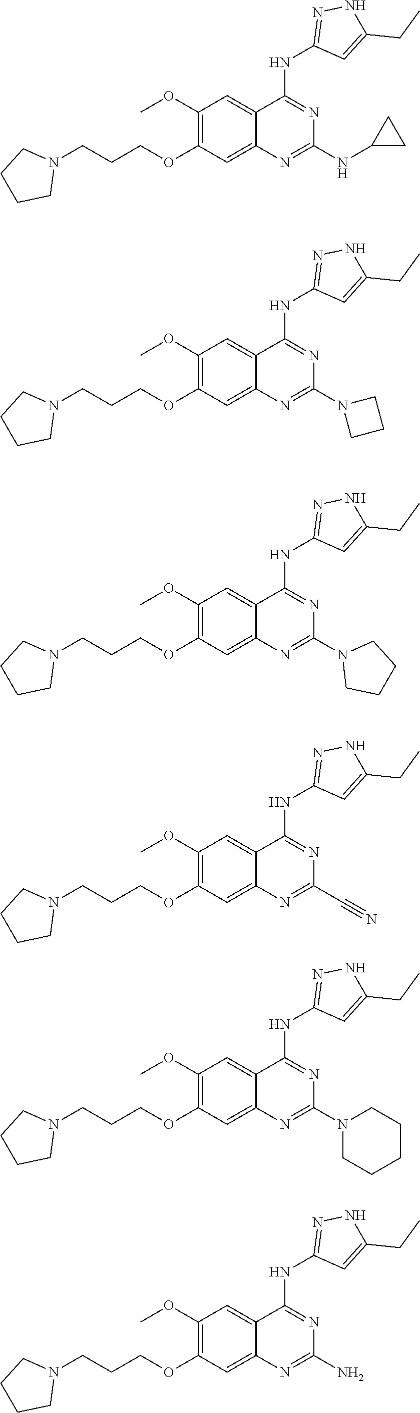

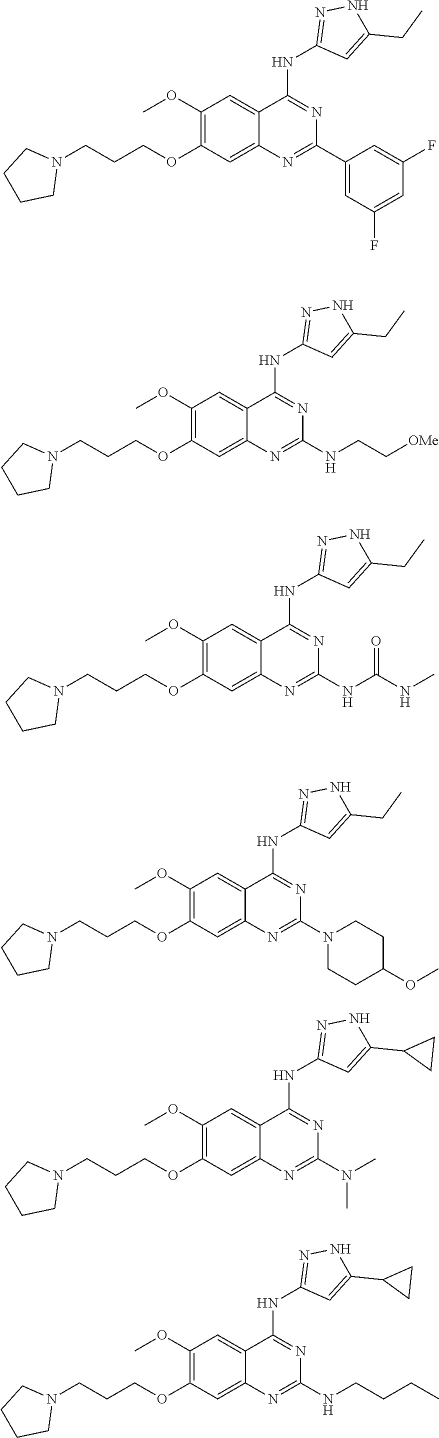

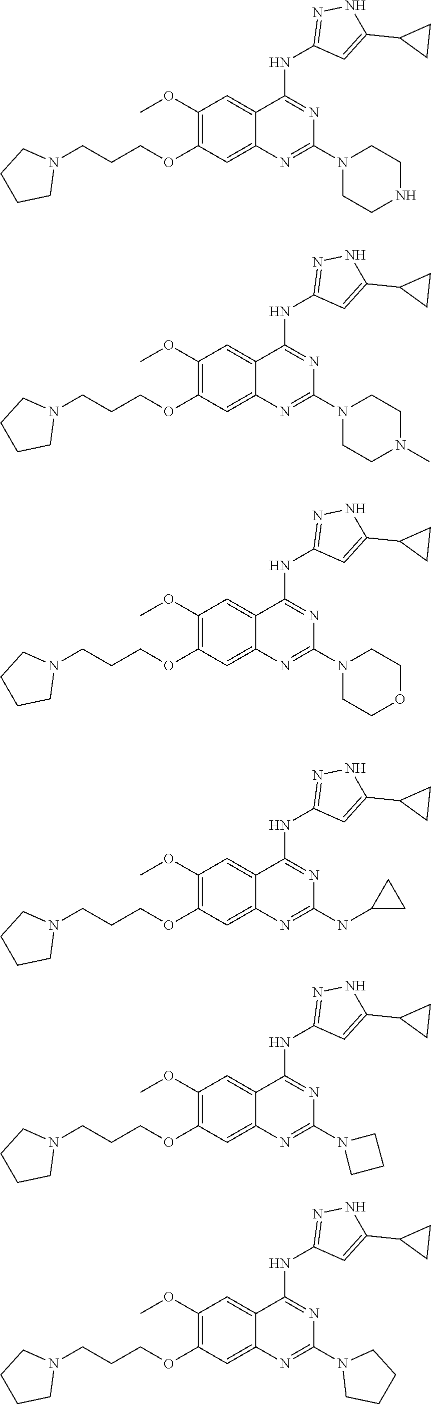

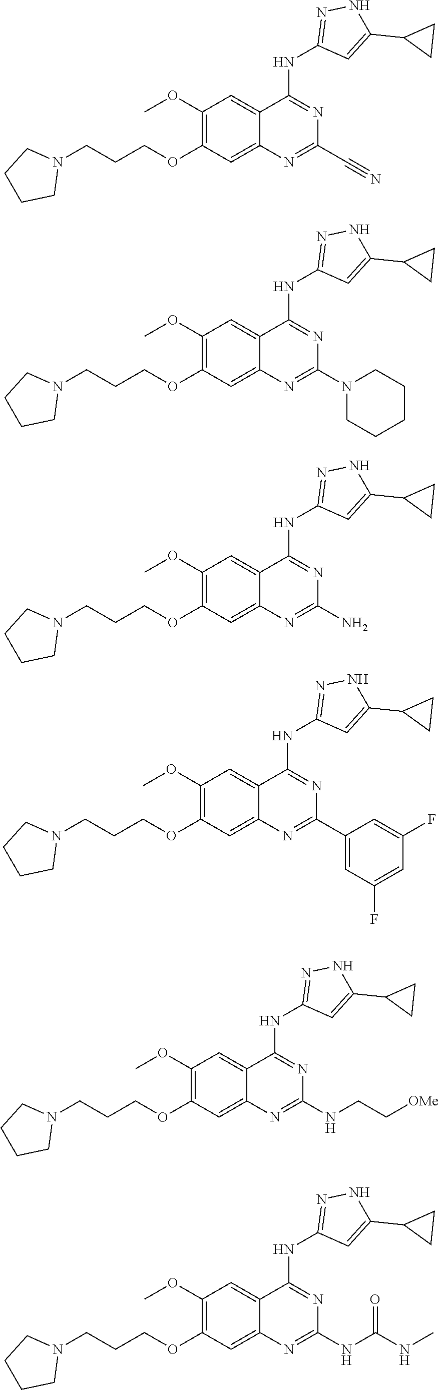

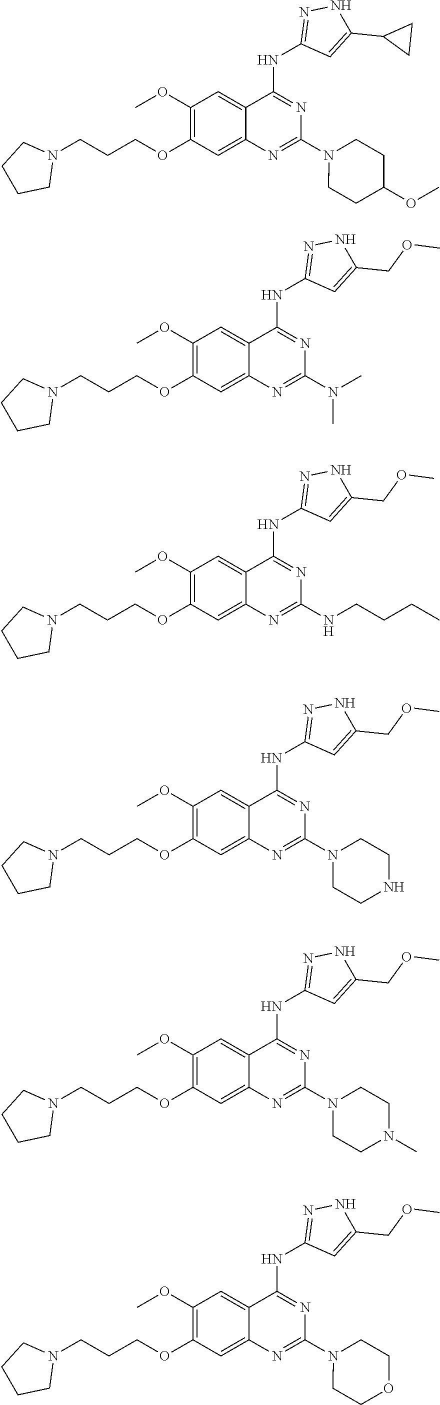

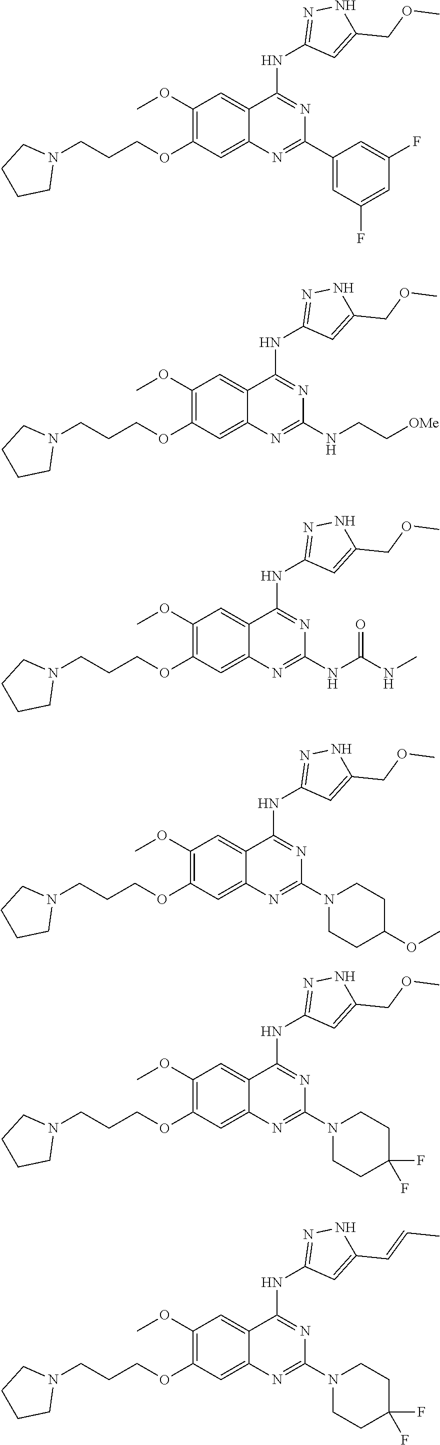

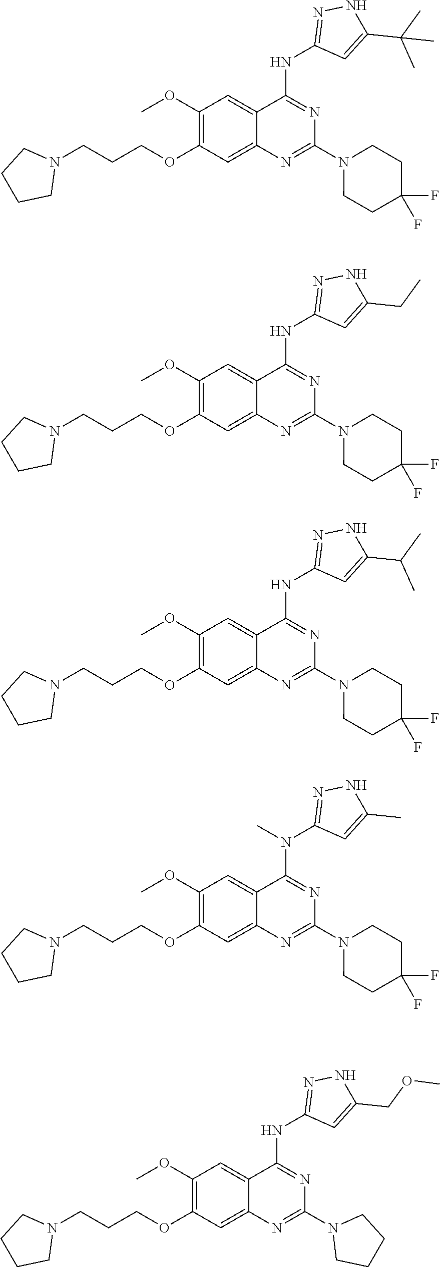

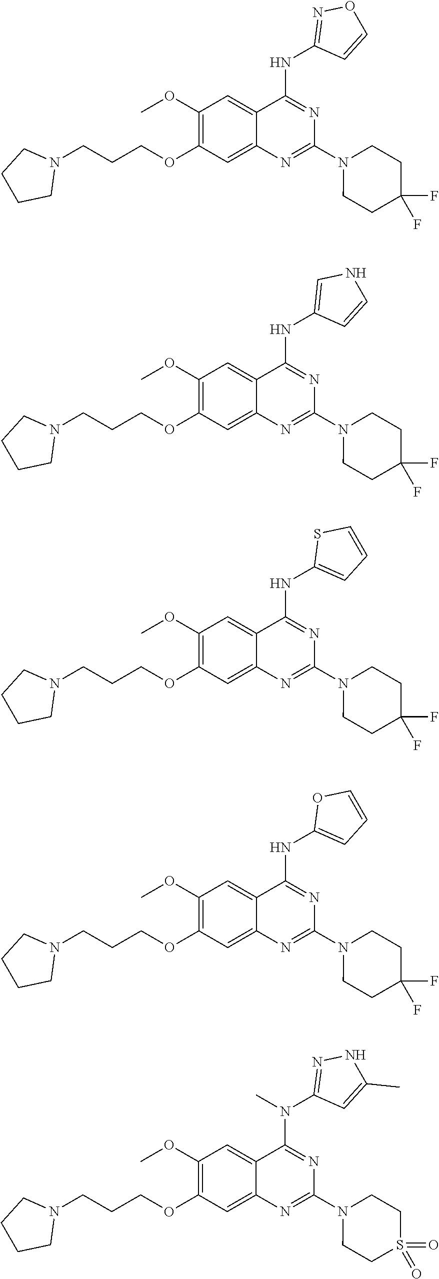 [SMILES: COc1cc2c(Nc3ccon3)nc(N3CCC(F)(F)CC3)nc2cc1OCCCN1CCCC1 ; COc1cc2c(Nc3cc[nH]c3)nc(N3CCC(F)(F)CC3)nc2cc1OCCCN1CCCC1 ; COc1cc2c(Nc3cccs3)nc(N3CCC(F)(F)CC3)nc2cc1OCCCN1CCCC1 ; COc1cc2c(Nc3ccco3)nc(N3CCC(F)(F)CC3)nc2cc1OCCCN1CCCC1 ; COc1cc2c(N(C)c3cc(C)[nH]n3)nc(N3CCS(=O)(=O)CC3)nc2cc1OCCCN1CCCC1]

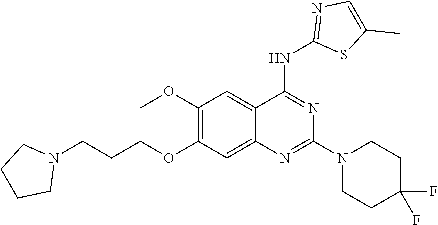 [SMILES: COc1cc2c(Nc3ncc(C)s3)nc(N3CCC(F)(F)CC3)nc2cc1OCCCN1CCCC1]

In several embodiments, the disclosed quinazolinyl compounds target directly and/or inhibit one or more protein kinases. In several embodiments, the disclosed quinazolinyl compounds can be especially advantageous in the context of the treatment of kinase-related diseases. In several embodiments, the disclosed quinazolinyl compounds can be especially advantageous in the context of the treatment of kinase-related cancers, autoimmune disease, and Duchenne muscular dystrophy. In several embodiments, the compounds as disclosed herein are characterized by their ability to bind one or more of protein kinases to treat and/or prevent cancer, autoimmune disease, and Duchenne muscular dystrophy (DMD). In several embodiments, the protein kinase is selected from the group consisting of CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and LTK. In several embodiments, the protein kinase is selected from the group consisting of abl, Akt. Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck. IGF-1R, INS-R, Jak, KDR, Lck. Lyn, MEK, p38, PDGFR, PIK, PKC, PYK2, Ros, Tie1, Tie2. Trk, Yes, and Zap70. In several embodiments, the protein kinase is selected from the group consisting of CLK1, CLK4, PLK4, FLT3, JNK1. In several embodiments, the disclosed quinazolinyl compounds target directly and/or inhibit protein kinases involved in the mitogen activated protein kinase (MAPK) signaling pathway. In several embodiments, the disclosed quinazolinyl compounds target directly and/or inhibit lipid kinases (e.g., to treat cancer, autoimmune disease, and/or DMD). In several embodiments, the disclosed quinazolinyl compounds target directly and/or inhibit lipid kinases (e.g. PI3K) constitute a separate group of kinases with structural similarity to protein kinases.

### BRIEF DESCRIPTION OF THE DRAWINGS

FIGS. 1A-1E provide various embodiments disclosed herein. FIG. 1A provides the general structure of a quinazolinyl structure, which may be substituted with any one of Substituent A at position 2. Substituent B at position 4, Substituent C at position 6, and Substituent D at position 7.

FIG. 1B provides a list of Substituent A structures. This list is nonlimiting and Substituent A may include other structures (as disclosed elsewhere herein). Any of these substituents may be further optionally substituted by replacing a —H atom for a substituent.

FIG. 1C provides a list of Substituent B structures. This list is nonlimiting and Substituent B may include other structures (as disclosed elsewhere herein). Any of these substituents may be further optionally substituted by replacing a —H atom for a substituent.

FIG. 1D provides a list of Substituent C structures. This list is nonlimiting and Substituent C may include other structures (as disclosed elsewhere herein). Any of these substituents may be further optionally substituted by replacing a —H atom for a substituent.

FIG. 1E provides a list of Substituent D structures. This list is nonlimiting and Substituent D may include other structures (as disclosed elsewhere herein). Any of these substituents may be further optionally substituted by replacing a —H atom for a substituent.

FIG. 2 provides an exemplary reaction scheme for a method of making a structure of Formula (I).

FIG. 3A provides results of studies of tumor growth rates in test subjects treated with an embodiment of the quinazolinyl compounds disclosed herein.

FIG. 3B provides results of studies of tumor mass in test subjects treated with an embodiment of the quinazolinyl compounds disclosed herein.

### DETAILED DESCRIPTION

Several embodiments disclosed herein pertain to quinazolinyl compounds, methods of using quinazolinyl compounds (e.g., for kinase inhibition and/or to treat kinase related disorders), compositions comprising quinazolinyl compounds, and methods of making quinazolinyl compounds. In several embodiments, a quinazoline compound comprises a quinazoline core. In several embodiments, the quinazoline compound comprises an amine heteroaryl substituent bonded to the quinazoline ring at the 4-position. In several embodiments, the quinazoline compound comprises a methoxy group at the 6-position. In several embodiments, the quinazoline compound comprises an alkoxy group at the 7-position. In several embodiments, the quinazoline compound comprises a pendant cyclic group (e.g., a five member heterocyclic group, such as a pyrrolidine) at the 7-position, connected to the bicycle either with an alkyne or an alkoxy group. In several embodiments, quinazolinyl structures as disclosed herein may be used in to inhibit kinases and/or for the treatment of kinase-related disorders. The following description provides context and examples, but should not be interpreted to limit the scope of the inventions covered by the claims that follow in this specification or in any other application that claims priority to this specification. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” (or “substituted or unsubstituted”) if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine(alkyl), a di-substituted amine(alkyl), a diamino-group, a polyamino, a diether-group, and a polyether-.

In some embodiments, substituted group(s) is (are) substituted with one or more substituent(s) individually and independently selected from C₁-C₄ alkyl, amino, hydroxy, and halogen.

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—. (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{x} and R^{y} of an NR^{x}R^{y} group are indicated to be “taken together,” it means that they are covalently bonded to one another to form a ring:

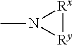

Likewise, when two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

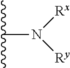

and R^{x} and R^{y} are defined as selected from the group consisting of hydrogen and alkyl, or R^{x} and R^{y} together with the nitrogen to which they are attached form a heterocyclyl (or R^{x} and R^{y} “taken together” form a heterocyclyl), it is meant that R^{x} and R^{y} can be selected from hydrogen or alkyl, or alternatively, the substructure has structure:

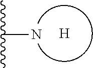

where ring H is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

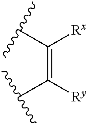

and R^{x} and R^{y} are defined as selected from the group consisting of hydrogen and alkyl, or R^{x} and R^{y} together with the atoms to which they are attached form an aryl or carbocyclyl (or R^{x} and R^{y} “taken together” form a carbocyclyl), it is meant that R^{x} and R^{y} can be selected from hydrogen or alkyl, or alternatively, the substructure has structure:

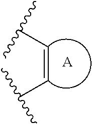

where A is an aryl ring or a carbocyclyl containing the depicted double bond.

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 20 carbon atoms (whenever it appears herein, a numerical range such as “1 to 20” refers to each integer in the given range; e.g., “1 to 20 carbon atoms” means that the alkyl group may consist of 1, 2, 3, 4, 5, 6, 7, 8, 9, 10, 11, 12, 13, 14, 15, 16, 17, 18, 19, or 20 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted. By way of example only, “C₁-C₅ alkyl” indicates that there are 1 to 5 carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

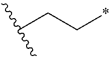 [SMILES: *CC*]

to represent ethylene. The alkylene group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group

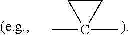

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted. The alkenyl group may have 2 to 20 carbon atoms (whenever it appears herein, a numerical range such as “2 to 20” refers to each integer in the given range; e.g., “2 to 20 carbon atoms” means that the alkenyl group may consist of 2 carbon atoms, 3 carbon atoms, etc., up to and including 20 carbon atoms). The alkenyl group may also be a medium size alkenyl having 2 to 12 carbon atoms. The alkenyl group could also be a lower alkenyl having 2 to 6 carbon atoms. An alkenyl group may be represented in the same manner as used for an alkyl. For example, a “C₂-C₅ alkenyl” includes 1-propenyl (e.g., a “C₃ alkenyl”), 1-butenyl (e.g., a “C₄ alkenyl”), 2-butenyl (e.g., a “C₄ alkenyl”), 1-pentenyl (e.g., a “C₅ alkenyl”), and the like.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted. The alkynyl group may have 2 to 20 carbon atoms (whenever it appears herein, a numerical range such as “2 to 20” refers to each integer in the given range; e.g., “2 to 20 carbon atoms” means that the alkynyl group may consist of 2 carbon atoms, 3 carbon atoms, etc., up to and including 20 carbon atoms). The alkynyl group may also be a medium size alkynyl having 2 to 12 carbon atoms. The alkynyl group could also be a lower alkynyl having 2 to 6 carbon atoms. An alkynyl group may be represented in the same manner as used for an alkyl. For example, a “C₂-C₅ alkynyl” includes 1-propynyl (e.g., a “C₃ alkynyl”), 1-butynyl (e.g., a “C₄ alkynyl”), 2-butynyl (e.g., a “C₄ alkynyl”), 1-pentynyl (e.g., a “C₅ alkynyl”), and the like.

As used herein, “cycloalkyl” refers to a completely saturated (no double or triple bonds) mono- or multi-cyclic (such as bicyclic) hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic (such as bicyclic) hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “aryl” refers to a carbocyclic (all carbon) monocyclic or multicyclic (such as bicyclic) aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted. As used herein, “heteroaryl” refers to a monocyclic or multicyclic (such as bicyclic) aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heteroalkyl” refers to a straight or branched hydrocarbon chain (e.g., alkyl) containing one or more heteroatoms. A heteroatom is given its plain and ordinary meaning in organic chemistry, which includes an element other than carbon, including but not limited to, nitrogen (e.g., amino, mono-substituted amine, di-substituted amine, etc.), oxygen (e.g., alkoxy, ether, hydroxyl, etc.), sulfur, and halogens. The heteroalkyl group may have 1 to 20 carbon atoms although the present definition also covers the occurrence of the term “heteroalkyl” where no numerical range is designated. The heteroalkyl group may also be a medium size heteroalkyl having 1 to 12 carbon atoms. The heteroalkyl group could also be a lower heteroalkyl having 1 to 6 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain. A heteroalkyl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioamides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrroledione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “cycloalkyl(alkyl)” refer to an cycloalkyl group connected, as a substituent, via a lower alkylene group. The lower alkylene and cycloalkyl group of a cycloalkyl(alkyl) may be substituted or unsubstituted.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer to a heterocyclic or a heteroalicyclic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted. An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl), or heterocyclyl(alkyl); or R_{A} and R_{B} taken together form a heteroaryl or heterocycle. An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-carbamyl” group refers to an “—OC(═O)N(R_{A})—R” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “carbamide” group refers to an “—NHC(═O)N(R_{A})—R” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A carbamide may be substituted or unsubstituted.

An “carbonate” group refers to an “—OC(═O)O—R” group in which R can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A carbonate may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl); or R_{A} and R_{B} taken together form a heteroaryl or heterocycle. An O-carbamyl may be substituted or unsubstituted. An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl); or R_{A} and R_{B} taken together form a heteroaryl or heterocycle. A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl); or R_{A} and R_{B} taken together form a heteroaryl or heterocycle. An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

As used herein, a “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

As used herein, “nitro” group refers to an “—NO₂” group.

As used herein, a “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

As used herein, a “sulfinyl” group refers to an “—S(═O)—R” group in which R can be the same as defined with respect to sulfenyl. A sulfinyl may be substituted or unsubstituted.

As used herein, a “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The terms “amino” and “unsubstituted amino” as used herein refer to a —NH₂ group.

As used herein, a “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. A mono-substituted amine group can include, for example, a mono-alkylamine group, a mono-C₁-C₆ alkylamine group, a mono-arylamine group, a mono-C₆-C₁₀ arylamine group and the like. Examples of mono-substituted amine groups include, but are not limited to, —NH(methyl), —NH(phenyl) and the like.

As used herein, a “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl); or R_{A} and R_{B} taken together form a heteroaryl or heterocycle. R_{A} and R_{B} can independently be substituted or unsubstituted. A di-substituted amine group can include, for example, a di-alkylamine group, a di-C₁-C₆ alkylamine group, a di-arylamine group, a di-C₆-C₁₀ arylamine group and the like. Examples of di-substituted amine groups include, but are not limited to, —N(methyl)₂, −N(phenyl)(methyl), —N(ethyl)(methyl) and the like.

As used herein, “mono-substituted amine(alkyl)” group refers to a mono-substituted amine as provided herein connected, as a substituent, via a lower alkylene group. A mono-substituted amine(alkyl) may be substituted or unsubstituted. A mono-substituted amine(alkyl) group can include, for example, a mono-alkylamine(alkyl) group, a mono-C₁-C₆ alkylamine(C₁-C₆ alkyl) group, a mono-arylamine(alkyl group), a mono-C₆-C₁₀ arylamine(C₁-C₆ alkyl) group and the like. Examples of mono-substituted amine(alkyl) groups include, but are not limited to, —CH₂NH(methyl), —CH₂NH(phenyl), —CH₂CH₂NH(methyl), —CH₂CH₂NH(phenyl) and the like.

As used herein, “di-substituted amine(alkyl)” group refers to a di-substituted amine as provided herein connected, as a substituent, via a lower alkylene group. A di-substituted amine(alkyl) may be substituted or unsubstituted. A di-substituted amine(alkyl) group can include, for example, a dialkylamine(alkyl) group, a di-C₁-C₆ alkylamine(C₁-C₆ alkyl) group, a di-arylamine(alkyl) group, a di-C₆-C₁₀ arylamine(C₁-C₆ alkyl) group and the like. Examples of di-substituted amine(alkyl)groups include, but are not limited to, —CH₂N(methyl)₂. —CH₂N(phenyl)(methyl), —CH₂N(ethyl)(methyl), —CH₂CH₂N(methyl)₂, —CH₂CH₂N(phenyl)(methyl), —NCH₂CH₂(ethyl)(methyl) and the like.

As used herein, the term “diamino-” denotes an a “—N(R_{A})R_{B}—N(R_{C})(R_{D})” group in which R_{A}. R_{C}, and R_{D} can be independently a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “N” groups and can be (independently of R_{A}, R_{C}, and R_{D}) a substituted or unsubstituted alkylene group. R_{A}, R_{B}, R_{C}, and R_{D} can independently further be substituted or unsubstituted.

As used herein, the term “polyamino” denotes a “—(N(R_{A})R_{B}—)ₙ—N(R_{C})(R_{D})”. For illustration, the term polyamino can comprise —N(R_{A})alkyl-N(R_{A})alkyl-N(R_{A})alkyl-N(R_{A})alkyl-H. In some embodiments, the alkyl of the polyamino is as disclosed elsewhere herein. While this example has only 4 repeat units, the term “polyamino” may consist of 1, 2, 3, 4, 5, 6, 7, 8, 9, or 10 repeat units. R_{A}. R_{C}, and R_{D} can be independently a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “N” groups and can be (independently of R_{A}. R_{C}, and R_{D}) a substituted or unsubstituted alkylene group. R_{A}, R_{C}, and R_{D} can independently further be substituted or unsubstituted. As noted here, the polyamino comprises amine groups with intervening alkyl groups (where alkyl is as defined elsewhere herein).

As used herein, the term “ether” denotes a repeating -alkyl-O-alkyl group. For illustration, the term ether can comprise —(C₁-C₆ alkyl)-O—(C₁-C₆ alkyl). In some embodiments, the alkyl of the polyether is as disclosed elsewhere herein. As used herein, the term “diether-” denotes an a “—OR_{B}O—R_{A}” group in which R_{A} can be a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “O” groups and can be a substituted or unsubstituted alkylene group. R_{A} can independently further be substituted or unsubstituted.

As used herein, the term “polyether” denotes a repeating —(OR_{B}—)ₙOR_{A} group. For illustration, the term polyether can comprise -Oalkyl-Oalkyl-Oalkyl-Oalkyl-OR_{A}. In some embodiments, the alkyl of the polyether is as disclosed elsewhere herein. While this example has only 4 repeat units, the term “polyether” may consist of 1, 2, 3, 4, 5, 6, 7, 8, 9, or 10 repeat units. R_{A} can be a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{B} can be a substituted or unsubstituted alkylene group. R_{A} can independently further be substituted or unsubstituted. As noted here, the polyether comprises ether groups with intervening alkyl groups (where alkyl is as defined elsewhere herein and can be optionally substituted).

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms. As another example, C₂-C₆ alkynyl can include one, two, or three triple bonds.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

Two substituents may come together with the atom or atoms to which they are attached to form a ring that is spiro or fused with the rest of the compound.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

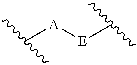

includes the substituent being oriented such that the A is attached at the leftmost attachment point of the molecule as well as the case in which A is attached at the rightmost attachment point of the molecule.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof. It is understood that, in any compound described herein having one or more chiral centers, all possible diastereomers are also envisioned. It is understood that, in any compound described herein all tautomers are envisioned. It is also understood that, in any compound described herein, all isotopes of the included atoms are envisioned. For example, any instance of hydrogen, may include hydrogen-1 (protium), hydrogen-2 (deuterium), hydrogen-3 (tritium) or other isotopes; any instance of carbon may include carbon-12, carbon-13, carbon-14, or other isotopes; any instance of oxygen may include oxygen-16, oxygen-17, oxygen-18, or other isotopes; any instance of fluorine may include one or more of fluorine-18, fluorine-19, or other isotopes; any instance of sulfur may include one or more of sulfur-32, sulfur-34, sulfur-35, sulfur-36, or other isotopes.

As used herein, the term “inhibitor” means any compound, molecule or composition that inhibits or reduces the activity of a target biomolecule. The inhibition can be achieved by, for example, blocking phosphorylation of the target (e.g., competing with adenosine triphosphate (ATP), a phosphorylating entity), by binding to a site outside the active site, affecting its activity by a conformational change, or by depriving kinases of access to the molecular chaperoning systems on which they depend for their cellular stability, leading to their ubiquitylation and degradation.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety. Quinazolinyl compounds as disclosed herein may be modified as prodrugs that release a quinazolinyl compound once inside the body of a subject.

The term “pro-drug ester” refers to derivatives of the compounds disclosed herein (e.g., quinazolinyl compounds) formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in U.S. Pat. No. 4,783,443A. Johnston et al., published Sep. 11, 1987 (incorporated by reference herein in its entirety).

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects.

The terms “treatment,” “treating.” “treat” and the like shall be given its ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including.” “containing.” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably.” “preferred.” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “or” should not be read as requiring mutual exclusivity among that group, but rather should be read as “and/or” unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Introduction

A number of diseases result from the improper regulation of the normal processes, including those that control cell division, differentiation, and apoptotic cell death. Protein kinases play a critical role in these regulatory processes. Kinase inhibitors have been used to treat diseases, such as cancer (e.g., by inhibiting mitotic processes). However, despite the fact that various inhibitors of kinases are known, there remains a need for selective inhibitors to be used for the treatment of diseases such as hyper-proliferative diseases, which offer one or more advantages over current compounds. Those advantages include: improved activity and/or efficacy; beneficial kinase selectivity profile according to the respective therapeutic need; improved side effect profile, such as fewer undesired side effects, lower intensity of side effects, or reduced (cyto)toxicity; improved targeting of mutant receptors in diseased cells; improved physicochemical properties, such as solubility/stability in water, body fluids, and/or pharmaceutical formulations; improved pharmacokinetic properties, allowing e.g. for dose reduction or an easier dosing scheme; easier drug substance manufacturing e.g. by shorter synthetic routes or easier purification. Several embodiments disclosed herein pertain to compounds that achieve one or more of these advantages (or others). Several embodiments disclosed herein pertain to compounds that address one or more deficiencies of known drug substances.

Disclosed herein are kinase inhibitors that a disrupt kinase activity and or inhibit protein kinases. In several embodiments, disclosed herein are quinazolinyl compounds, methods of using quinazolinyl compounds, compositions comprising quinazolinyl compounds, and methods of treatment using quinazolinyl compounds. In several embodiments, the disclosed quinazolinyl compounds are kinase inhibitors. In several embodiments, the disclosed quinazolinyl compounds are useful in methods of treating cancer, autoimmune disease, and/or DMD.

In several embodiments, the disclosed quinazolinyl compounds target directly and/or inhibit one or more protein kinases selected from the group consisting of CLK1. CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and LTK. In several embodiments, the disclosed quinazolinyl compounds target directly and/or inhibit one or more protein kinases selected from the group consisting of abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, and Zap70. In several embodiments, the disclosed quinazolinyl compounds target directly and/or inhibit protein kinases involved in the mitogen activated protein kinase (MAPK) signaling pathway. In several embodiments, the disclosed quinazolinyl compounds target directly and/or inhibit lipid kinases. In several embodiments, the disclosed quinazolinyl compounds target directly and/or inhibit lipid kinases (e.g. PI3K) constitute a separate group of kinases with structural similarity to protein kinases.

### Compounds

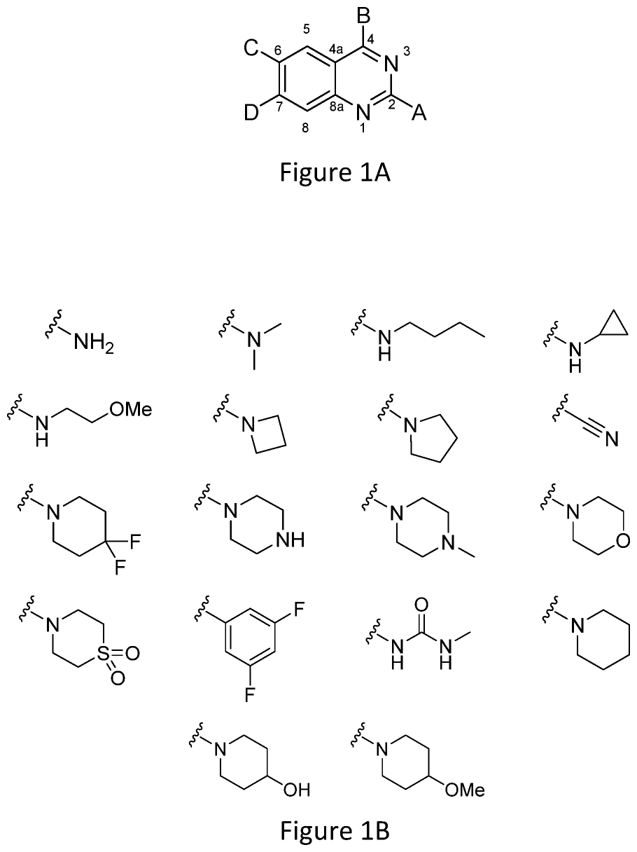

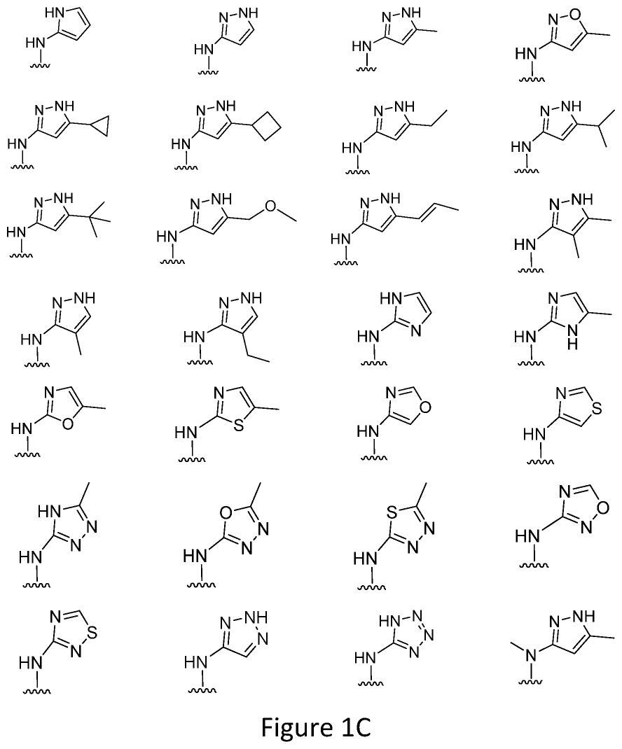

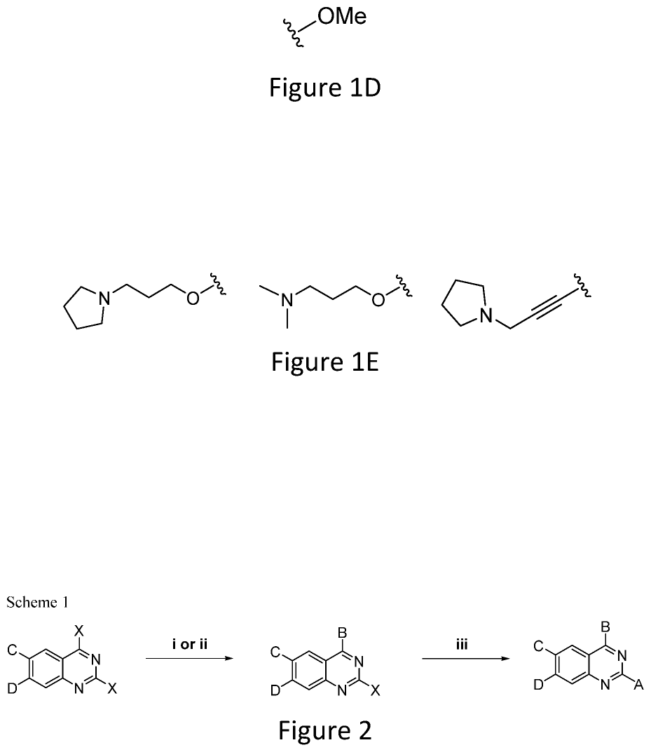

As disclosed elsewhere herein, several embodiments pertain to quinazolinyl compounds. In several embodiments, the quinazolinyl compounds are represented by one or more of Formulae (I), (IA), (IB), or (IC). In several embodiments, the quinazolinyl compound is represented by any one or more of the structures of FIGS. 1A-1E.

### Compounds of Formula (I)

Several embodiments pertain to quinazolinyl compounds having the structure of Formula (I) (or a stereoisomer, tautomer or pharmaceutically acceptable salt thereof):

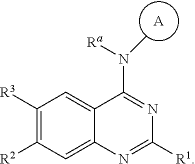

In several embodiments, R¹ is selected from the group consisting of optionally substituted 6-10 membered aryl, optionally substituted 3-10 membered heterocyclyl, optionally substituted 5-10 membered heteroaryl, optionally substituted carbamide, —CN, and —NR⁴R⁵. In several embodiments, each of R⁴ and R⁵ is independently selected from hydrogen, optionally substituted C₁-C₆ alkyl, or optionally substituted C₃-C₆ carbocyclyl; or alternatively, R⁴ and R⁵ taken together form an optionally substituted 3-10 membered heterocyclyl. In several embodiments, R² is —OR⁶ or optionally substituted (heterocyclyl)alkynyl. In several embodiments, R⁶ is selected from the group consisting of methyl, optionally substituted 2-10 membered heteroalkyl, and (heterocyclyl)alkyl. In several embodiments, R³ is selected from the group consisting of hydrogen, halogen, and C₁₋₆ alkoxy. In several embodiments, R^{a} is hydrogen or optionally substituted C₁-C₁₀ alkyl. In several embodiments, the A ring is an optionally substituted heteroaryl.

As disclosed elsewhere herein, in several embodiments, the A ring is an optionally substituted heteroaryl. In several embodiments, the A ring is an optionally substituted heteroaryl having 5 ring members. In several embodiments, the A ring comprises 1, 2, 3, or 4 heteroatoms. In several embodiments, when the A ring comprises one or more optional substituents, the optional substituents are as disclosed elsewhere herein. In several embodiments, when the A ring comprises one or more optional substituents, the one or more optional substitutions may be independently selected from the group consisting of optionally substituted C₁-C₁₀ alkyl, optionally substituted C₁-C₁₀ alkenyl, optionally substituted C₃-C₆ carbocyclyl, optionally substituted ether (e.g., optionally substituted —(C₁-C₆ alkyl)-O—(C₁-C₆ alkyl)), halo, cyano, hydroxy, and C₁-C₃ alkoxy. In several embodiments, when the A ring comprises one or more optional substituents, the one or more optional substitutions may be independently selected from the group consisting of optionally substituted C₁-C₁₀ alkyl and optionally substituted C₃-C₆ carbocyclyl. In several embodiments, when the A ring comprises one or more optional substituents, the one or more optional substitutions may be independently selected from the group consisting of C₁-C₃ alkyl, C₁-C₄ carbocyclyl, halo, cyano, hydroxy, and C₁-C₃ alkoxy. In several embodiments, when the A ring comprises one or more optional substituents, the one or more optional substitutions may be independently selected from the group consisting of C₁-C₃ alkyl and C₁-C₄ carbocyclyl.

In several embodiments, the A ring is represented by ring structure (AIa):

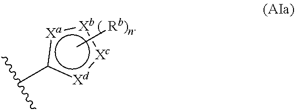

In several embodiments, each of X^{a}, X^{b}, X^{c}, and X^{d} are independently selected from the group consisting of C, N, O, and S. In several embodiments, any one or more of X^{a}, X^{b}, X^{c}, and X^{d} may be substituted by one or more R^{b} or H groups (e.g., where X^{a}, X^{b}, X^{c}, and/or X^{d} is a C or N atom). In several embodiments, each instance of R^{b}, where present, is independently selected from the group consisting of optionally substituted C₁-C₁₀ alkyl and optionally substituted C₃-C₆ carbocyclyl. In several embodiments, each instance of R^{b}, where present, is independently selected from the group consisting of optionally substituted C₁-C₃ alkyl, optionally substituted C₂-C₁₀ alkenyl, and optionally substituted C₃-C₄ carbocyclyl. In several embodiments, n is an integer selected from 0, 1, 2, 3, or 4. In several embodiments, n is 2. In several embodiments, n is 1. In several embodiments, n is 0. In several embodiments, each R^{b}, where present, replaces a —H bonded to a C or N atom within ring structure (AIa). In several embodiments, each instance of R^{b}, where present, is selected from the group consisting of:

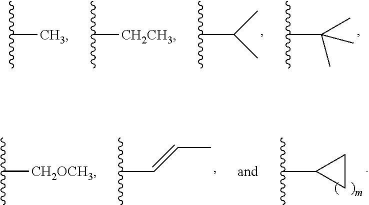

In several embodiments, m is an integer selected from 1, 2, 3, or 4. In several embodiments, m is an integer selected from 1 or 2. In several embodiments, each instance of R^{b}, where present, is selected from the group consisting of:

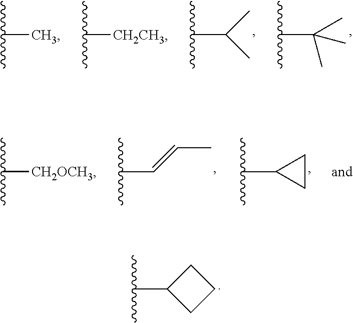

In several embodiments, ring structure (AIa) is further represented by a structure selected from the group consisting of:

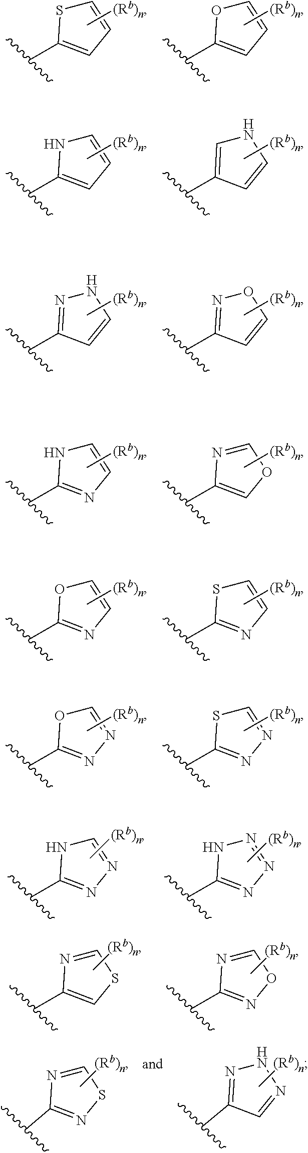

where each variable is as defined elsewhere herein. In several embodiments, as disclosed elsewhere herein, each R^{b}, where present, replaces a —H bonded to a C or N atom within ring structure (AIa).

In several embodiments, ring structure (AIa) is represented by a structure selected from the group consisting of:

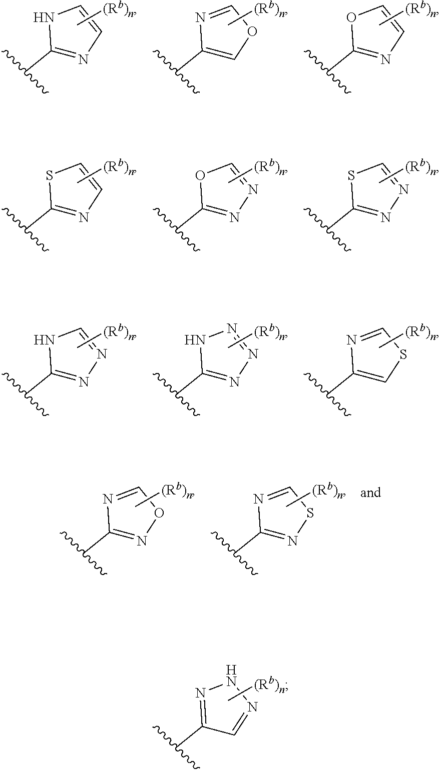

where each variable is as defined elsewhere herein. In several embodiments, as disclosed elsewhere herein, each R^{b}, where present, replaces a —H bonded to a C or N atom within ring structure (AIa).

In several embodiments, the A ring is a structure selected from the group consisting of the following structures:

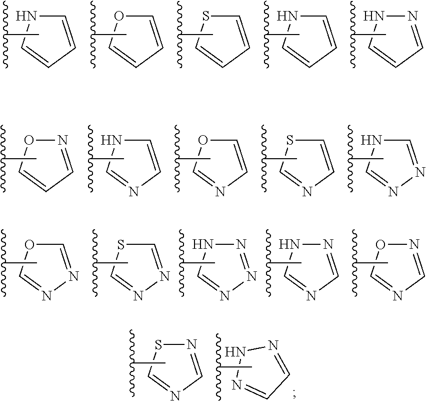

any one of which can be further optionally substituted by replacing one or more —H atoms of any carbon or nitrogen atom present with a substituent (such as optionally substituted C₁-C₁₀ alkyl and optionally substituted C₃-C₆ carbocyclyl).

In several embodiments, the A ring is a structure selected from the group consisting of:

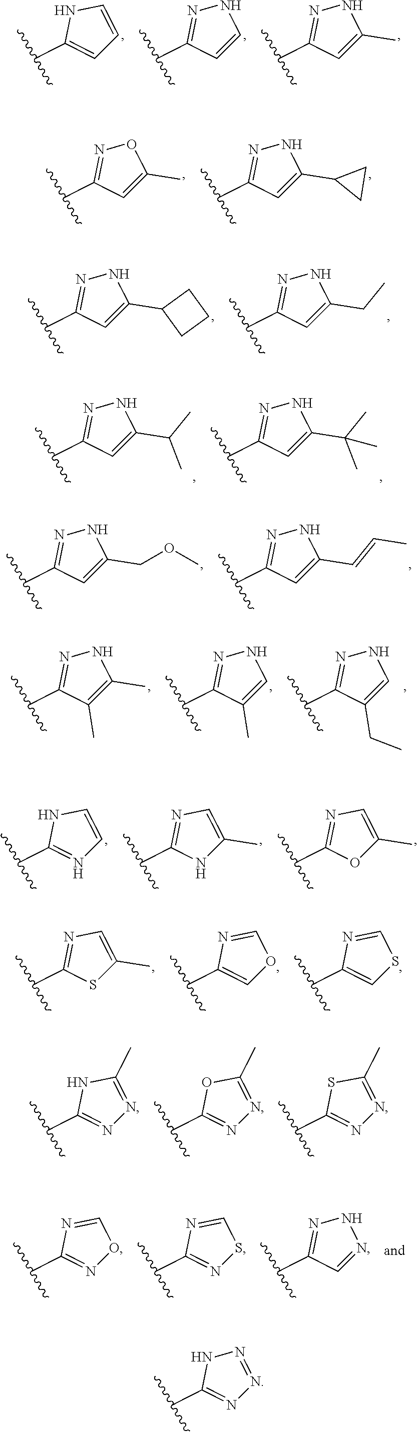

In some embodiments, the A ring is a structure is not represented by one or more of the following:

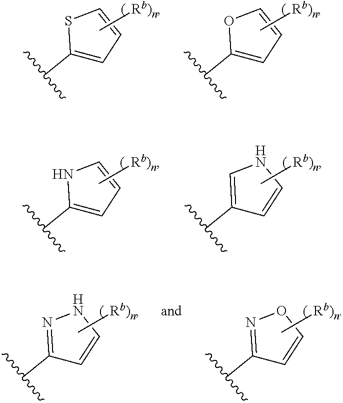

In some embodiments, the A ring is a structure is not represented by one or more of the following:

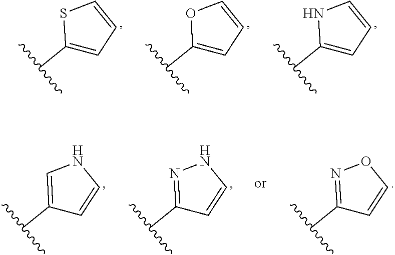 [SMILES: *c1cccs1 ; *c1ccco1 ; *c1ccc[nH]1 ; *c1cc[nH]c1 ; *c1cc[nH]n1 ; *c1ccon1]

As disclosed elsewhere herein, in several embodiments, R′ is selected from the group consisting of optionally substituted 6-10 membered aryl, optionally substituted 3-10 membered heterocyclyl, optionally substituted 5-10 membered heteroaryl, optionally substituted carbamide, —CN, and —NR⁴R⁵ (where R⁴ and R⁵ are as defined elsewhere herein). In several embodiments, where R¹ is a heteroaryl or heterocyclyl group, the ring may comprise 1, 2, 3, 4, or more heteroatoms. In several embodiments, R¹ is a structure selected from the group consisting of the following structures:

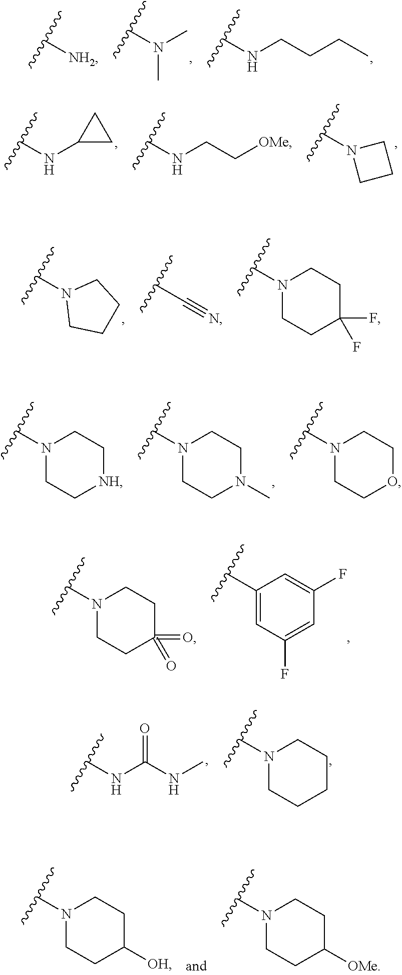

any one of which can be further optionally substituted by replacing one or more —H atoms of any carbon or nitrogen atom present with a substituent. In several embodiments, when an R¹ substituent comprises one or more optional substituents, the optional substituents are as disclosed elsewhere herein. In several embodiments, when an R¹ substituent comprises one or more optional substituents, the one or more optional substitutions may be independently selected from the group consisting of C₁-C₃ alkyl, halo, cyano, hydroxy, ether (e.g., —(C₁-C₆ alkyl)-O—(C₁-C₆ alkyl)), and C₁-C₃ alkoxy. In several embodiments, when an R¹ substituent comprises one or more optional substituents, the one or more optional substitutions may be independently selected from the group consisting amino, —OH, C₁-C₆ alkyl, and halogen.

As disclosed elsewhere herein, in several embodiments, R² is-OR⁶ or optionally substituted (heterocyclyl)alkynyl (where R⁶ is as disclosed elsewhere herein). In several embodiments, R² is a (heterocyclyl) alkynyl where the alkynyl is a C₂-C₆ alkynyl.

In several embodiments, R² is represented by the following structure:

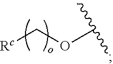

where R^{c} is a 3 to 8 member heterocyclyl having 1 to 2 heteroatoms or a 2-6 membered heteroalkyl having 1 to 2 heteroatoms. In several embodiments, R² is represented by the following structure:

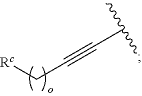

where R^{c} is a 3 to 8 member heterocyclyl having 1 to 2 heteroatoms. In several embodiments, R^{c} is a heterocyclyl with one heteroatom in the heterocyclic group. In several embodiments, R^{c} is represented by a structure as shown below:

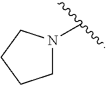

In several embodiments, R^{c} is a heteroalkyl with one heteroatom in the heteroalkyl group. In several embodiments, R^{c} is represented by a structure as shown below:

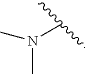

In several embodiments, o is an integer selected from 1, 2, 3, 4, or 5. In several embodiments, o is an integer selected from 1, 2, 3, or 4. In several embodiments, o is 3. In several embodiments, o is 1.

In several embodiments, R² is selected from the group consisting of:

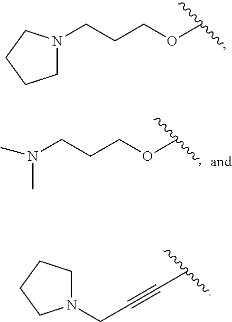

As disclosed elsewhere herein, in several embodiments, R³ is selected from the group consisting of hydrogen, halogen, and C₁₋₆ alkoxy. In several embodiments, R³ is —OMe.

As disclosed elsewhere herein, in several embodiments, R^{a} is hydrogen or optionally substituted C₁-C₁₀ alkyl. In several embodiments, R^{a} is hydrogen. In several embodiments, R^{a} is methyl.

In several embodiments of the structure of Formula (I), when R^{a} is —H, R³ is —OMe, R¹ is

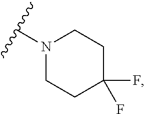 [SMILES: *N1CCC(F)(F)CC1]

and
R² is one of the following structures:

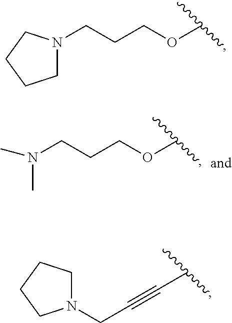

then the A-ring is not the following:

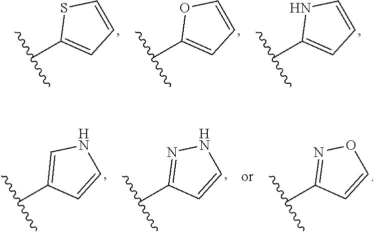 [SMILES: *c1cccs1 ; *c1ccco1 ; *c1ccc[nH]1 ; *c1cc[nH]c1 ; *c1cc[nH]n1 ; *c1ccon1]

In several embodiments, Formula (I) does not include any one of the following structures:

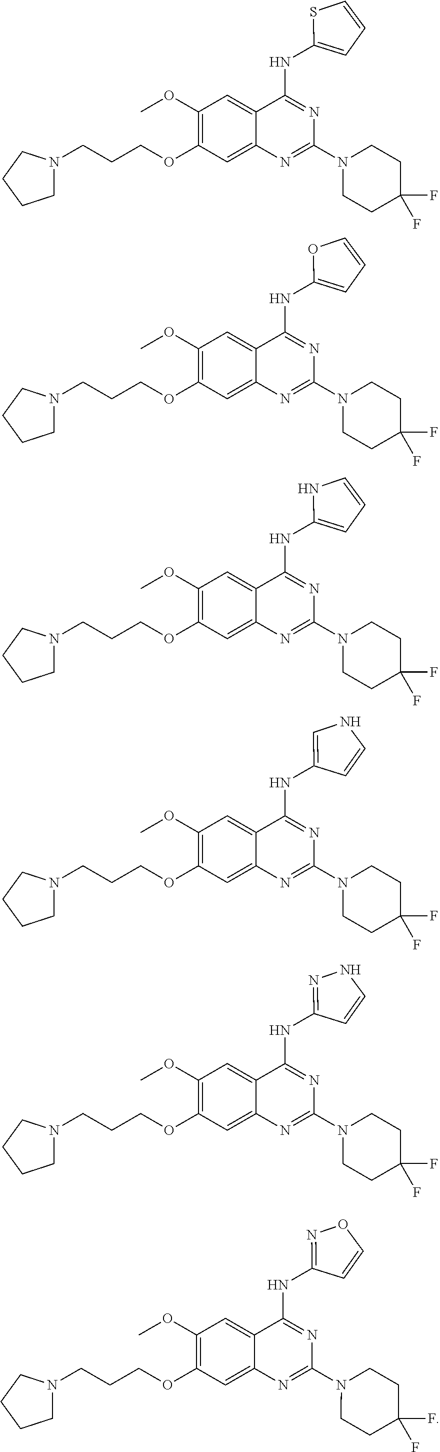

In several embodiments, Formula (I) does not include any one of the following structures:

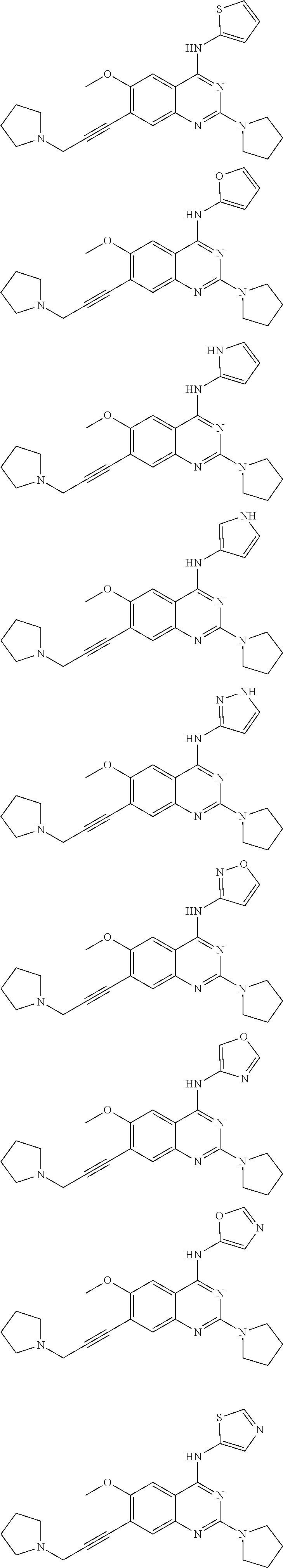

In several embodiments, Formula (I) does not include any one of the following structures:

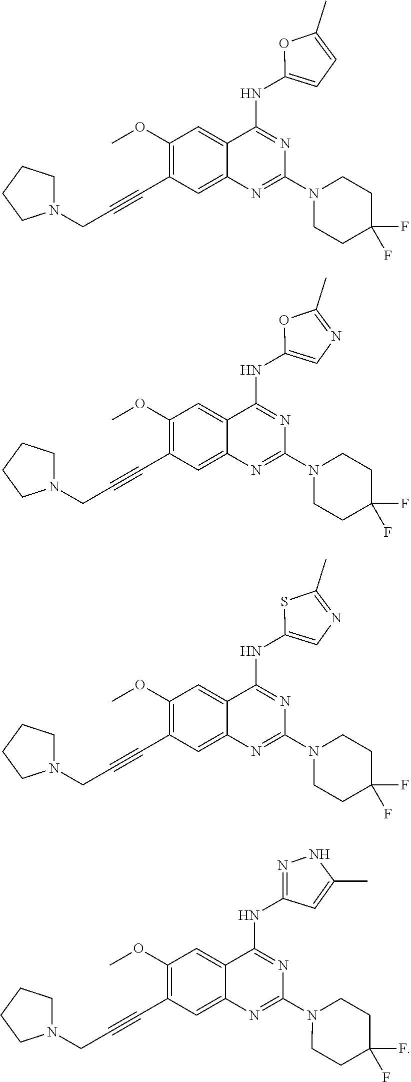

In several embodiments of the structure of Formula (I), when R² is:

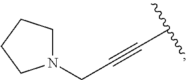

then R¹ is not:

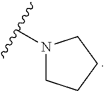 [SMILES: *N1CCCC1]

In several embodiments of the structure of Formula (I), when R² is:

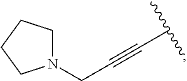

then R¹ is:

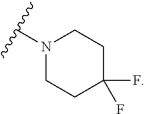 [SMILES: *N1CCC(F)(F)CC1]

In several embodiments, the compound of Formula (I) is represented by a compound selected from the group consisting of:

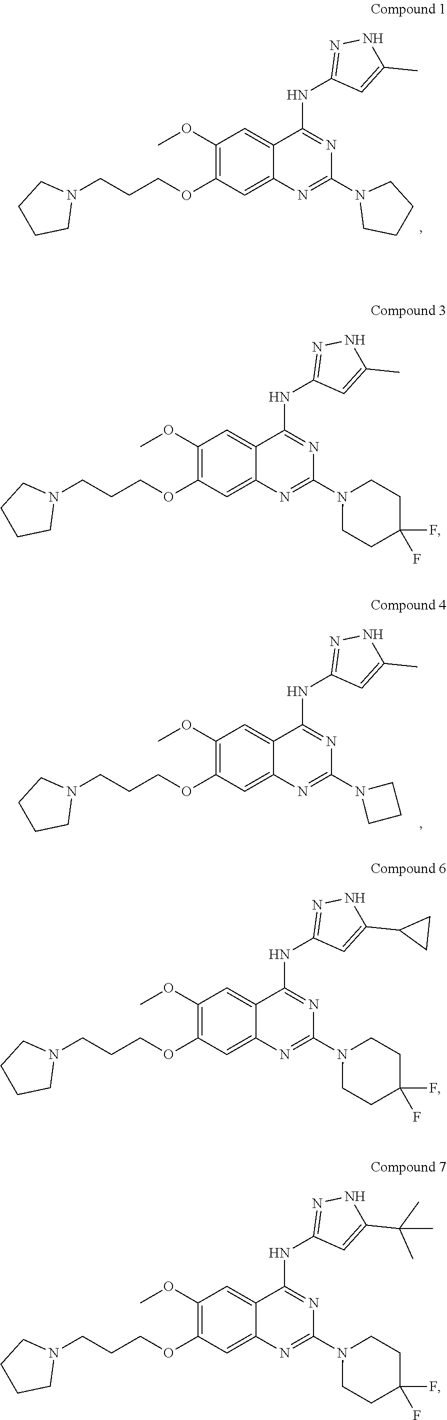 [SMILES: COc1cc2c(Nc3cc(C)[nH]n3)nc(N3CCCC3)nc2cc1OCCCN1CCCC1 ; COc1cc2c(Nc3cc(C)[nH]n3)nc(N3CCC(F)(F)CC3)nc2cc1OCCCN1CCCC1 ; COc1cc2c(Nc3cc(C)[nH]n3)nc(N3CCC3)nc2cc1OCCCN1CCCC1 ; COc1cc2c(Nc3cc(C4CC4)[nH]n3)nc(N3CCC(F)(F)CC3)nc2cc1OCCCN1CCCC1 ; COc1cc2c(Nc3cc(C(C)(C)C)[nH]n3)nc(N3CCC(F)(F)CC3)nc2cc1OCCCN1CCCC1]

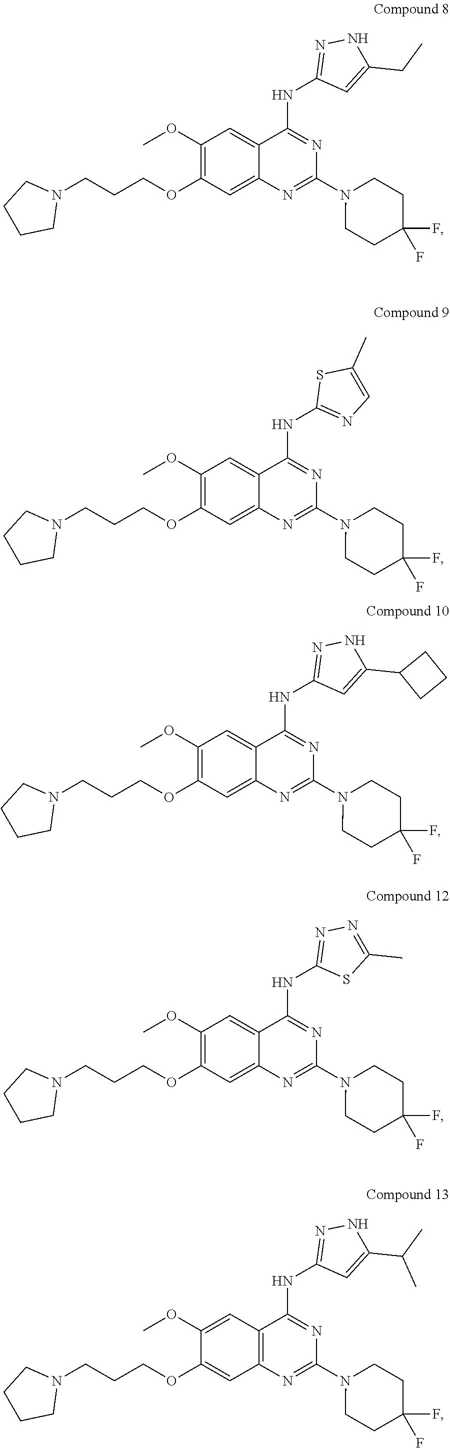 [SMILES: CCc1cc(Nc2nc(N3CCC(F)(F)CC3)nc3cc(OCCCN4CCCC4)c(OC)cc23)n[nH]1 ; COc1cc2c(Nc3ncc(C)s3)nc(N3CCC(F)(F)CC3)nc2cc1OCCCN1CCCC1 ; COc1cc2c(Nc3cc(C4CCC4)[nH]n3)nc(N3CCC(F)(F)CC3)nc2cc1OCCCN1CCCC1 ; COc1cc2c(Nc3nnc(C)s3)nc(N3CCC(F)(F)CC3)nc2cc1OCCCN1CCCC1 ; COc1cc2c(Nc3cc(C(C)C)[nH]n3)nc(N3CCC(F)(F)CC3)nc2cc1OCCCN1CCCC1]

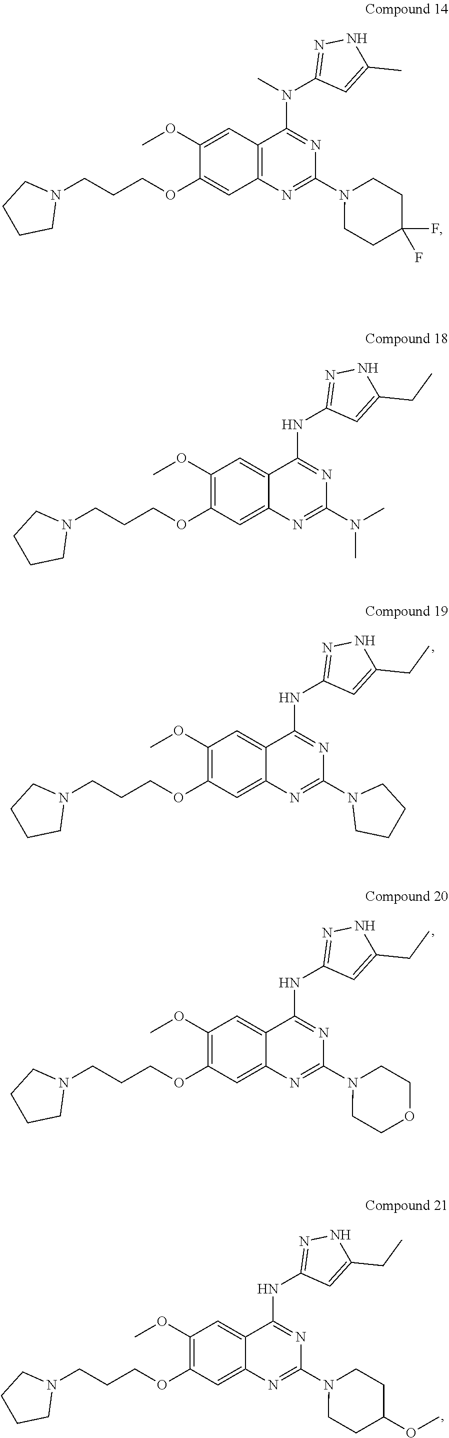 [SMILES: COc1cc2c(N(C)c3cc(C)[nH]n3)nc(N3CCC(F)(F)CC3)nc2cc1OCCCN1CCCC1 ; CCc1cc(Nc2nc(N(C)C)nc3cc(OCCCN4CCCC4)c(OC)cc23)n[nH]1 ; CCc1cc(Nc2nc(N3CCCC3)nc3cc(OCCCN4CCCC4)c(OC)cc23)n[nH]1 ; CCc1cc(Nc2nc(N3CCOCC3)nc3cc(OCCCN4CCCC4)c(OC)cc23)n[nH]1 ; CCc1cc(Nc2nc(N3CCC(OC)CC3)nc3cc(OCCCN4CCCC4)c(OC)cc23)n[nH]1]

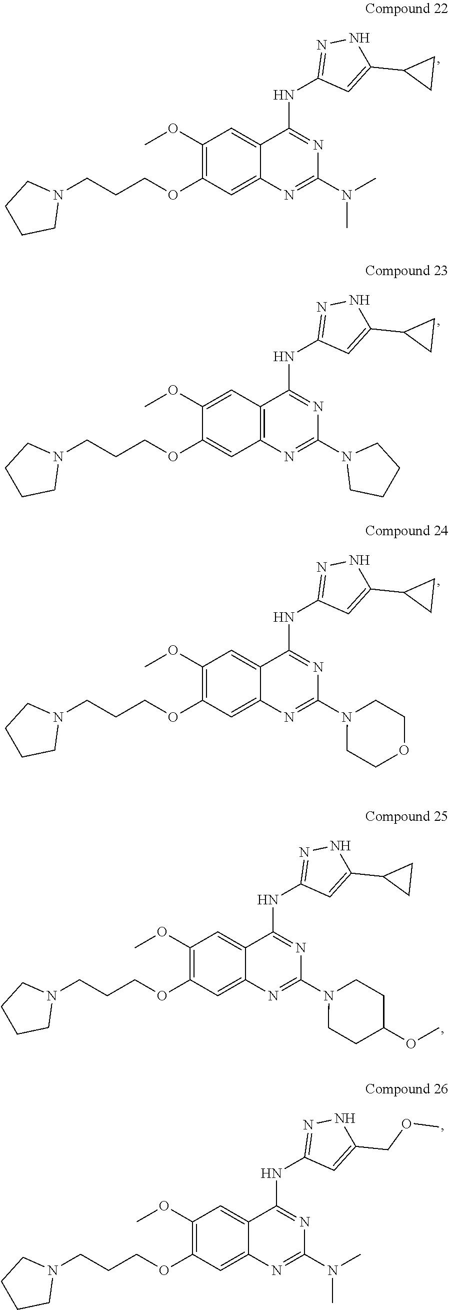 [SMILES: COc1cc2c(Nc3cc(C4CC4)[nH]n3)nc(N(C)C)nc2cc1OCCCN1CCCC1 ; COc1cc2c(Nc3cc(C4CC4)[nH]n3)nc(N3CCCC3)nc2cc1OCCCN1CCCC1 ; COc1cc2c(Nc3cc(C4CC4)[nH]n3)nc(N3CCOCC3)nc2cc1OCCCN1CCCC1 ; COc1cc2c(Nc3cc(C4CC4)[nH]n3)nc(N3CCC(OC)CC3)nc2cc1OCCCN1CCCC1 ; COCc1cc(Nc2nc(N(C)C)nc3cc(OCCCN4CCCC4)c(OC)cc23)n[nH]1]

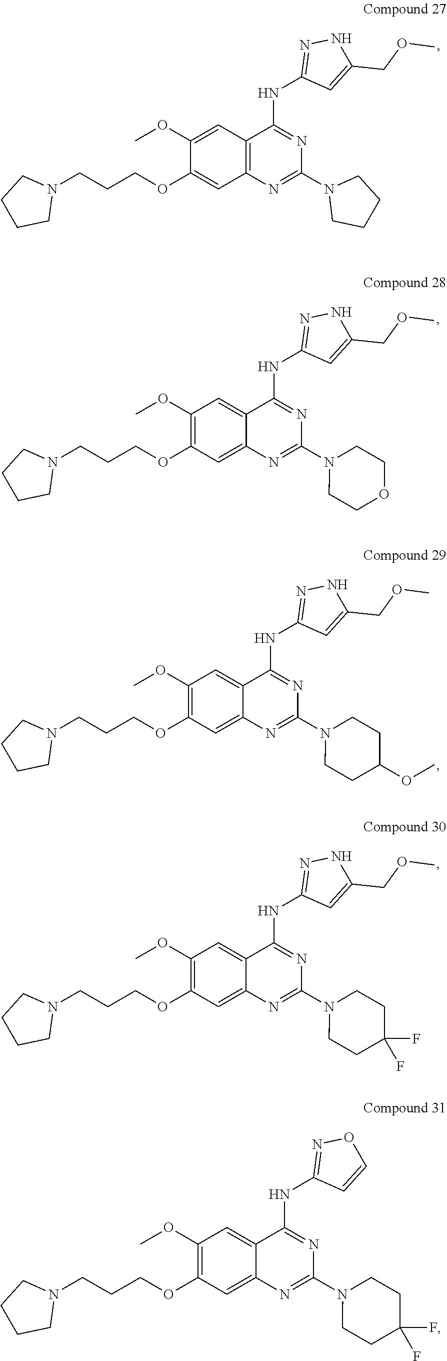 [SMILES: COCc1cc(Nc2nc(N3CCCC3)nc3cc(OCCCN4CCCC4)c(OC)cc23)n[nH]1 ; COCc1cc(Nc2nc(N3CCOCC3)nc3cc(OCCCN4CCCC4)c(OC)cc23)n[nH]1 ; COCc1cc(Nc2nc(N3CCC(OC)CC3)nc3cc(OCCCN4CCCC4)c(OC)cc23)n[nH]1 ; COCc1cc(Nc2nc(N3CCC(F)(F)CC3)nc3cc(OCCCN4CCCC4)c(OC)cc23)n[nH]1 ; COc1cc2c(Nc3ccon3)nc(N3CCC(F)(F)CC3)nc2cc1OCCCN1CCCC1]

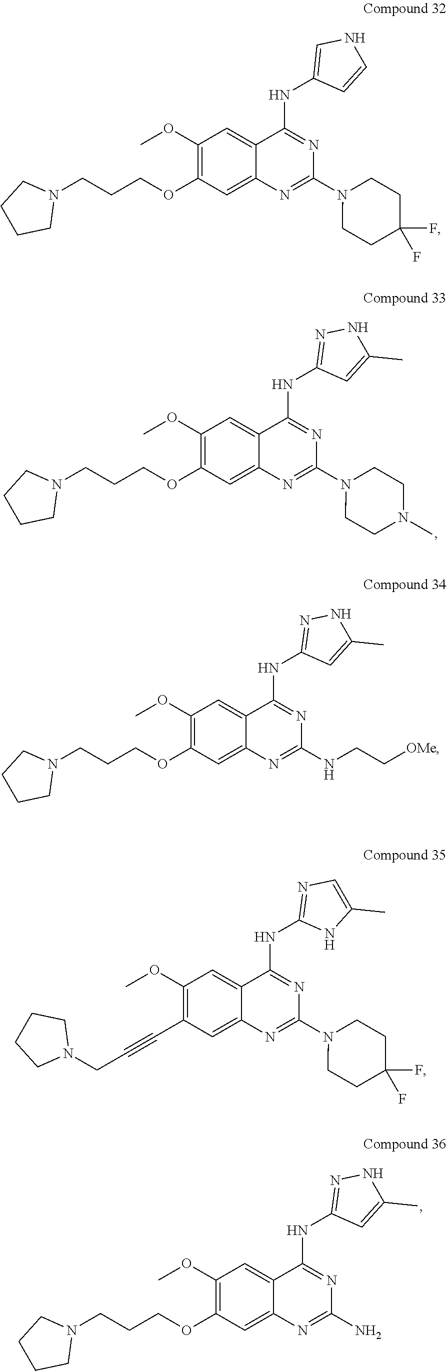 [SMILES: COc1cc2c(Nc3cc[nH]c3)nc(N3CCC(F)(F)CC3)nc2cc1OCCCN1CCCC1 ; COc1cc2c(Nc3cc(C)[nH]n3)nc(N3CCN(C)CC3)nc2cc1OCCCN1CCCC1 ; COCCNc1nc(Nc2cc(C)[nH]n2)c2cc(OC)c(OCCCN3CCCC3)cc2n1 ; COc1cc2c(Nc3ncc(C)[nH]3)nc(N3CCC(F)(F)CC3)nc2cc1C#CCN1CCCC1 ; COc1cc2c(Nc3cc(C)[nH]n3)nc(N)nc2cc1OCCCN1CCCC1]

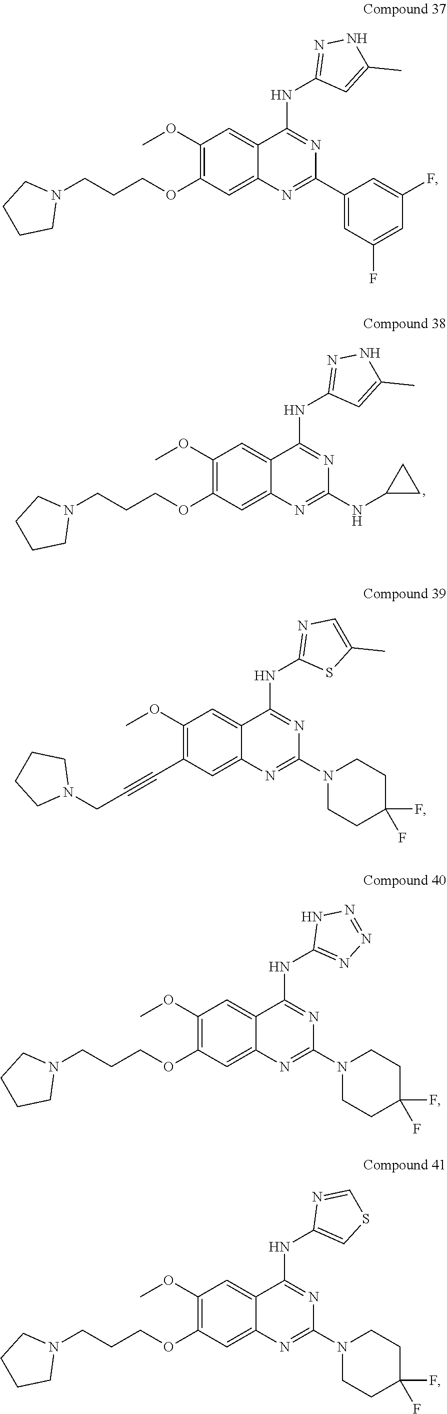 [SMILES: COc1cc2c(Nc3cc(C)[nH]n3)nc(-c3cc(F)cc(F)c3)nc2cc1OCCCN1CCCC1 ; COc1cc2c(Nc3cc(C)[nH]n3)nc(NC3CC3)nc2cc1OCCCN1CCCC1 ; COc1cc2c(Nc3ncc(C)s3)nc(N3CCC(F)(F)CC3)nc2cc1C#CCN1CCCC1 ; COc1cc2c(Nc3nnn[nH]3)nc(N3CCC(F)(F)CC3)nc2cc1OCCCN1CCCC1 ; COc1cc2c(Nc3cscn3)nc(N3CCC(F)(F)CC3)nc2cc1OCCCN1CCCC1]

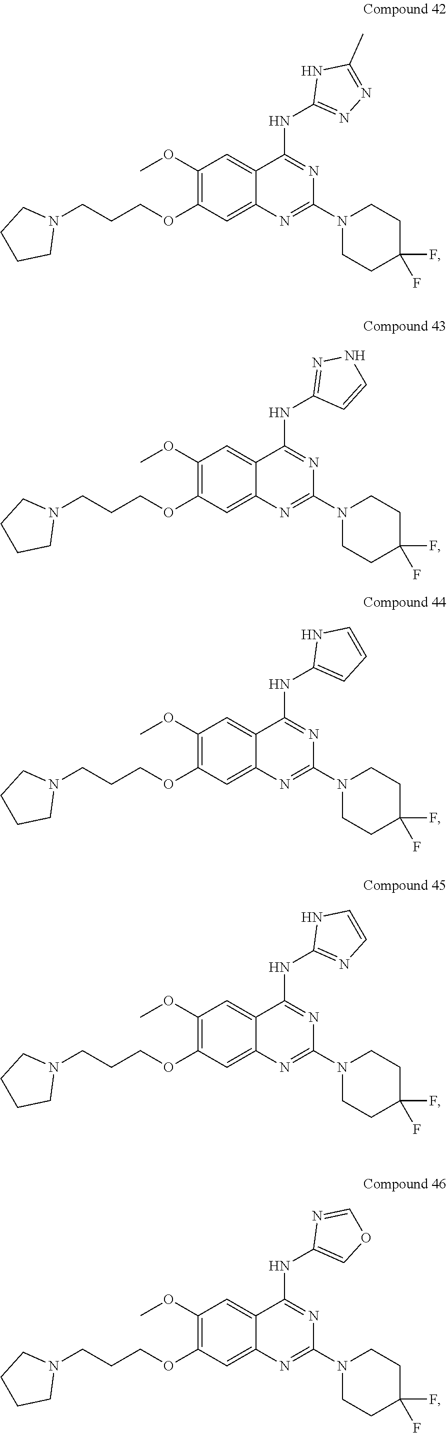 [SMILES: COc1cc2c(Nc3nnc(C)[nH]3)nc(N3CCC(F)(F)CC3)nc2cc1OCCCN1CCCC1 ; COc1cc2c(Nc3cc[nH]n3)nc(N3CCC(F)(F)CC3)nc2cc1OCCCN1CCCC1 ; COc1cc2c(Nc3ccc[nH]3)nc(N3CCC(F)(F)CC3)nc2cc1OCCCN1CCCC1 ; COc1cc2c(Nc3ncc[nH]3)nc(N3CCC(F)(F)CC3)nc2cc1OCCCN1CCCC1 ; COc1cc2c(Nc3cocn3)nc(N3CCC(F)(F)CC3)nc2cc1OCCCN1CCCC1]

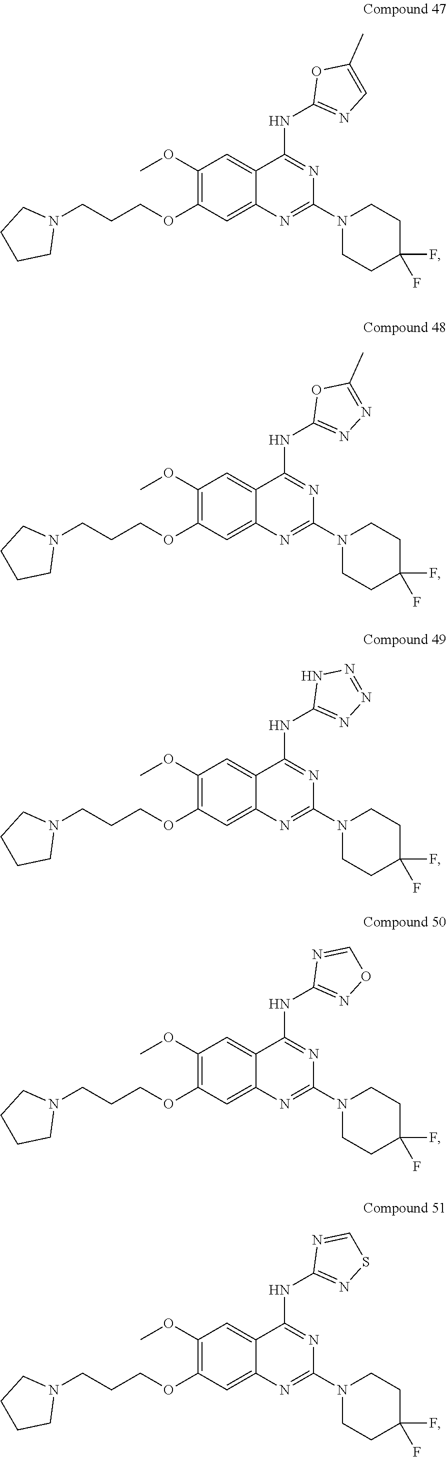 [SMILES: COc1cc2c(Nc3ncc(C)o3)nc(N3CCC(F)(F)CC3)nc2cc1OCCCN1CCCC1 ; COc1cc2c(Nc3nnc(C)o3)nc(N3CCC(F)(F)CC3)nc2cc1OCCCN1CCCC1 ; COc1cc2c(Nc3nnn[nH]3)nc(N3CCC(F)(F)CC3)nc2cc1OCCCN1CCCC1 ; COc1cc2c(Nc3ncon3)nc(N3CCC(F)(F)CC3)nc2cc1OCCCN1CCCC1 ; COc1cc2c(Nc3ncsn3)nc(N3CCC(F)(F)CC3)nc2cc1OCCCN1CCCC1]

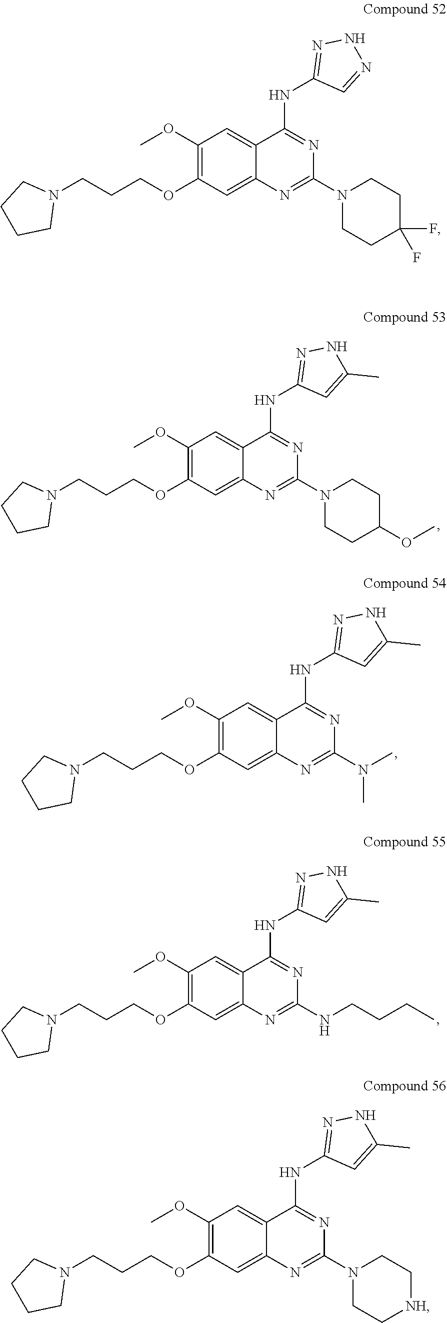 [SMILES: COc1cc2c(Nc3cn[nH]n3)nc(N3CCC(F)(F)CC3)nc2cc1OCCCN1CCCC1 ; COc1cc2c(Nc3cc(C)[nH]n3)nc(N3CCC(OC)CC3)nc2cc1OCCCN1CCCC1 ; COc1cc2c(Nc3cc(C)[nH]n3)nc(N(C)C)nc2cc1OCCCN1CCCC1 ; CCCCNc1nc(Nc2cc(C)[nH]n2)c2cc(OC)c(OCCCN3CCCC3)cc2n1 ; COc1cc2c(Nc3cc(C)[nH]n3)nc(N3CCNCC3)nc2cc1OCCCN1CCCC1]

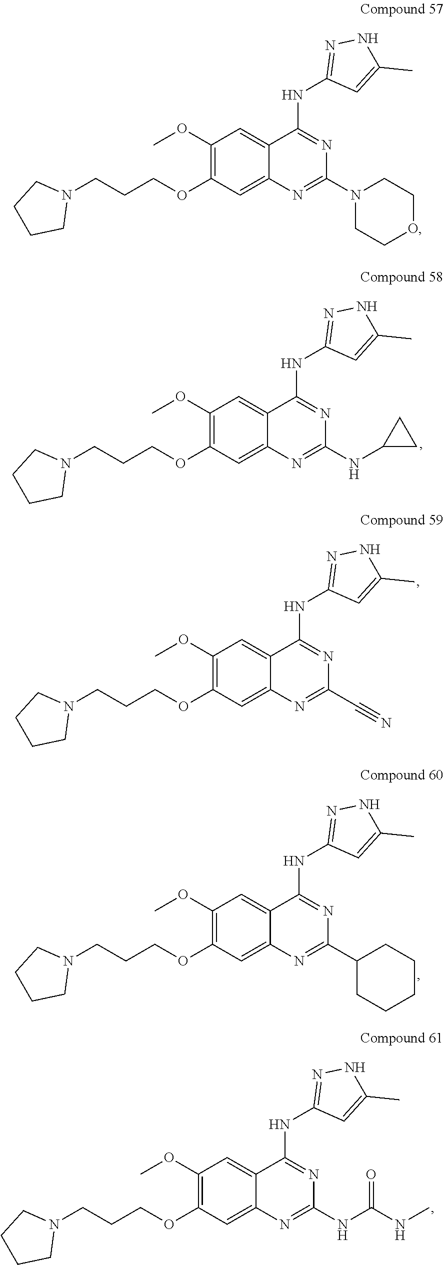 [SMILES: COc1cc2c(Nc3cc(C)[nH]n3)nc(N3CCOCC3)nc2cc1OCCCN1CCCC1 ; COc1cc2c(Nc3cc(C)[nH]n3)nc(NC3CC3)nc2cc1OCCCN1CCCC1 ; COc1cc2c(Nc3cc(C)[nH]n3)nc(C#N)nc2cc1OCCCN1CCCC1 ; COc1cc2c(Nc3cc(C)[nH]n3)nc(C3CCCCC3)nc2cc1OCCCN1CCCC1 ; CNC(=O)Nc1nc(Nc2cc(C)[nH]n2)c2cc(OC)c(OCCCN3CCCC3)cc2n1]

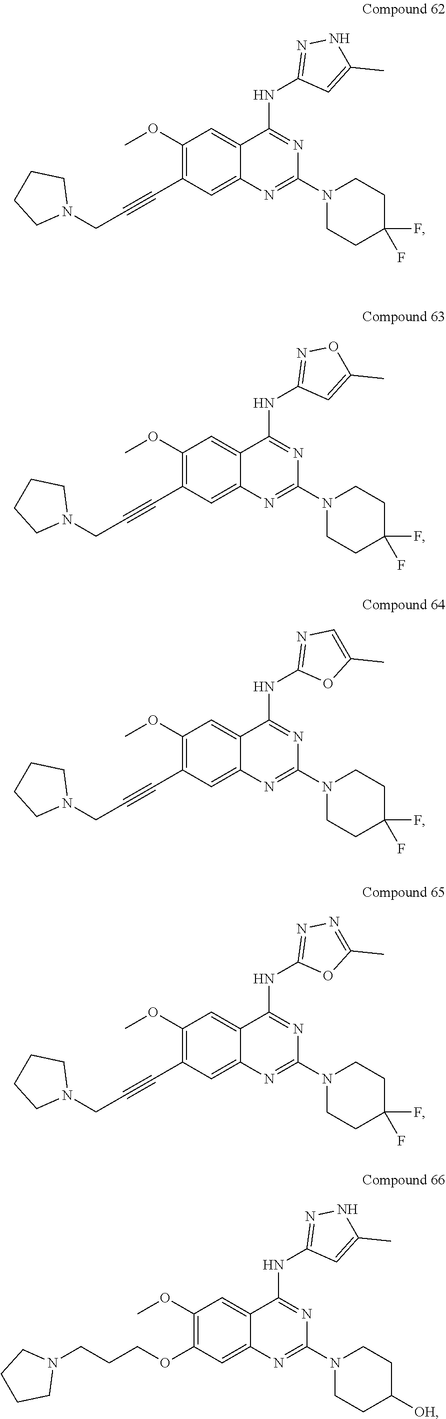 [SMILES: COc1cc2c(Nc3cc(C)[nH]n3)nc(N3CCC(F)(F)CC3)nc2cc1C#CCN1CCCC1 ; COc1cc2c(Nc3cc(C)on3)nc(N3CCC(F)(F)CC3)nc2cc1C#CCN1CCCC1 ; COc1cc2c(Nc3ncc(C)o3)nc(N3CCC(F)(F)CC3)nc2cc1C#CCN1CCCC1 ; COc1cc2c(Nc3nnc(C)o3)nc(N3CCC(F)(F)CC3)nc2cc1C#CCN1CCCC1 ; COc1cc2c(Nc3cc(C)[nH]n3)nc(N3CCC(O)CC3)nc2cc1OCCCN1CCCC1]

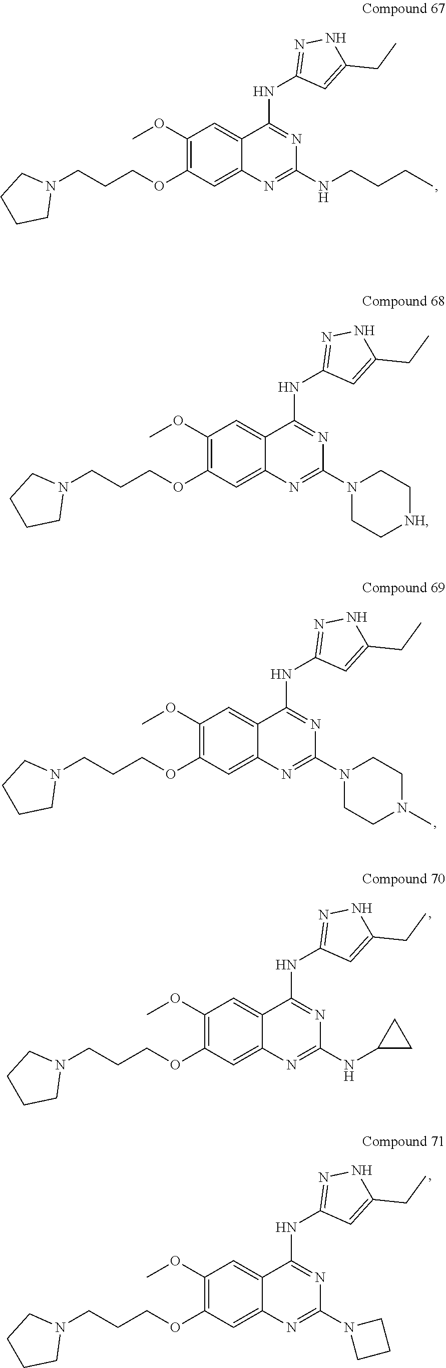 [SMILES: CCCCNc1nc(Nc2cc(CC)[nH]n2)c2cc(OC)c(OCCCN3CCCC3)cc2n1 ; CCc1cc(Nc2nc(N3CCNCC3)nc3cc(OCCCN4CCCC4)c(OC)cc23)n[nH]1 ; CCc1cc(Nc2nc(N3CCN(C)CC3)nc3cc(OCCCN4CCCC4)c(OC)cc23)n[nH]1 ; CCc1cc(Nc2nc(NC3CC3)nc3cc(OCCCN4CCCC4)c(OC)cc23)n[nH]1 ; CCc1cc(Nc2nc(N3CCC3)nc3cc(OCCCN4CCCC4)c(OC)cc23)n[nH]1]

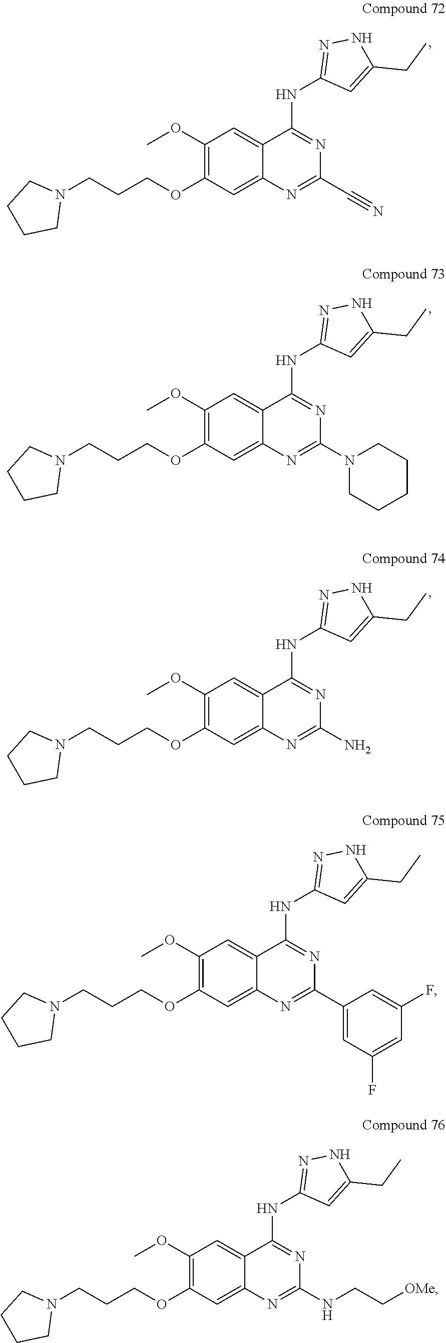 [SMILES: CCc1cc(Nc2nc(C#N)nc3cc(OCCCN4CCCC4)c(OC)cc23)n[nH]1 ; CCc1cc(Nc2nc(N3CCCCC3)nc3cc(OCCCN4CCCC4)c(OC)cc23)n[nH]1 ; CCc1cc(Nc2nc(N)nc3cc(OCCCN4CCCC4)c(OC)cc23)n[nH]1 ; CCc1cc(Nc2nc(-c3cc(F)cc(F)c3)nc3cc(OCCCN4CCCC4)c(OC)cc23)n[nH]1 ; CCc1cc(Nc2nc(NCCOC)nc3cc(OCCCN4CCCC4)c(OC)cc23)n[nH]1]

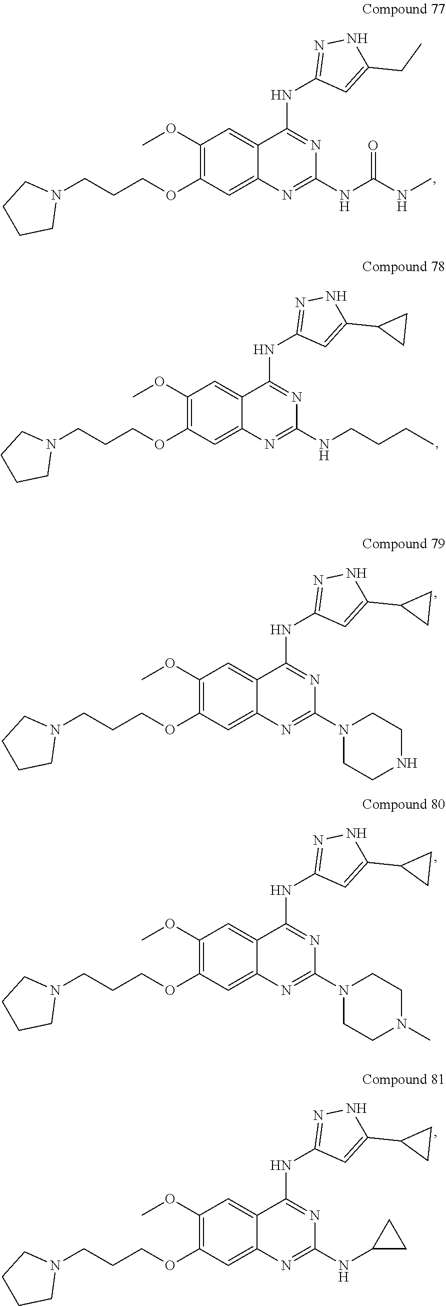 [SMILES: CCc1cc(Nc2nc(NC(=O)NC)nc3cc(OCCCN4CCCC4)c(OC)cc23)n[nH]1 ; CCCCNc1nc(Nc2cc(C3CC3)[nH]n2)c2cc(OC)c(OCCCN3CCCC3)cc2n1 ; COc1cc2c(Nc3cc(C4CC4)[nH]n3)nc(N3CCNCC3)nc2cc1OCCCN1CCCC1 ; COc1cc2c(Nc3cc(C4CC4)[nH]n3)nc(N3CCN(C)CC3)nc2cc1OCCCN1CCCC1 ; COc1cc2c(Nc3cc(C4CC4)[nH]n3)nc(NC3CC3)nc2cc1OCCCN1CCCC1]

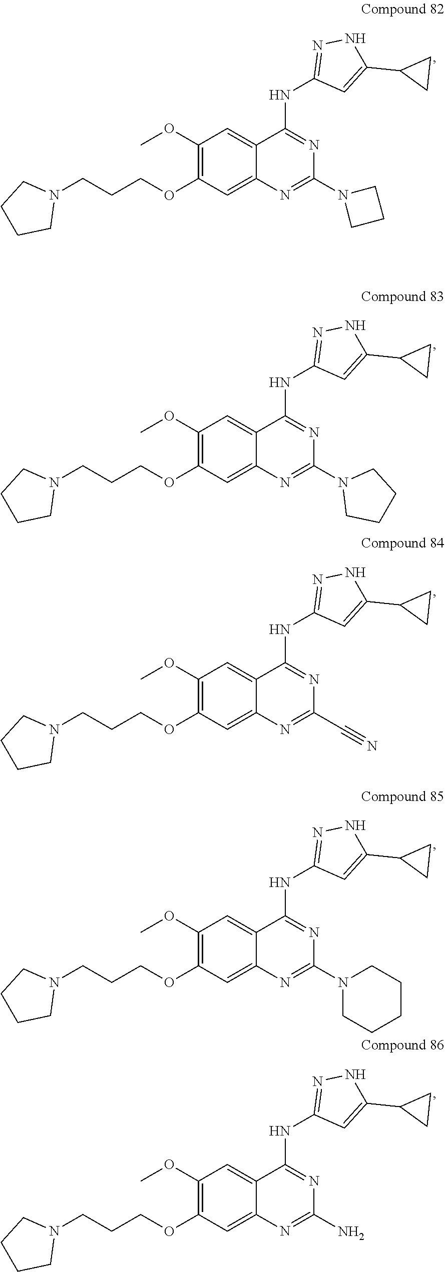 [SMILES: COc1cc2c(Nc3cc(C4CC4)[nH]n3)nc(N3CCC3)nc2cc1OCCCN1CCCC1 ; COc1cc2c(Nc3cc(C4CC4)[nH]n3)nc(N3CCCC3)nc2cc1OCCCN1CCCC1 ; COc1cc2c(Nc3cc(C4CC4)[nH]n3)nc(C#N)nc2cc1OCCCN1CCCC1 ; COc1cc2c(Nc3cc(C4CC4)[nH]n3)nc(N3CCCCC3)nc2cc1OCCCN1CCCC1 ; COc1cc2c(Nc3cc(C4CC4)[nH]n3)nc(N)nc2cc1OCCCN1CCCC1]

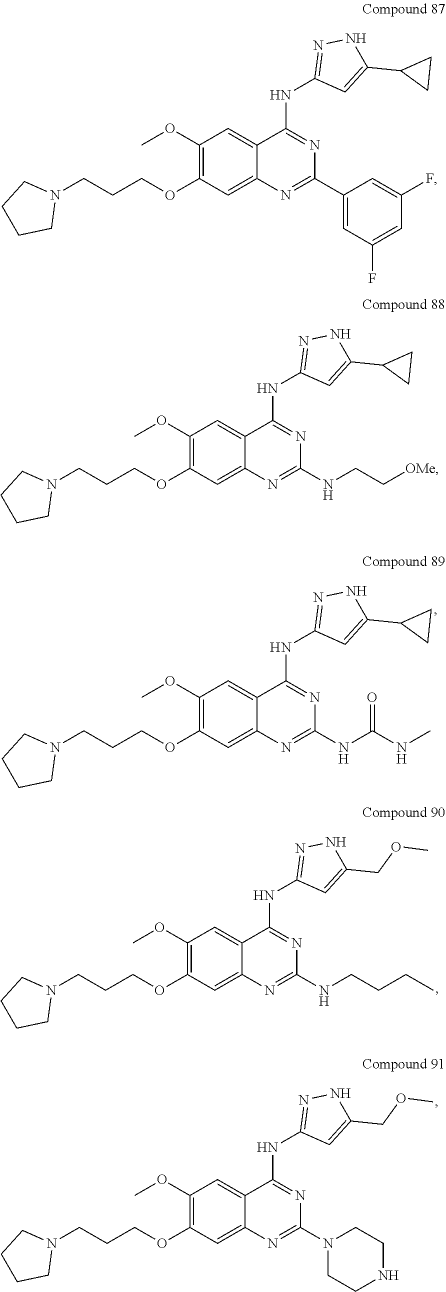 [SMILES: COc1cc2c(Nc3cc(C4CC4)[nH]n3)nc(-c3cc(F)cc(F)c3)nc2cc1OCCCN1CCCC1 ; COCCNc1nc(Nc2cc(C3CC3)[nH]n2)c2cc(OC)c(OCCCN3CCCC3)cc2n1 ; CNC(=O)Nc1nc(Nc2cc(C3CC3)[nH]n2)c2cc(OC)c(OCCCN3CCCC3)cc2n1 ; CCCCNc1nc(Nc2cc(COC)[nH]n2)c2cc(OC)c(OCCCN3CCCC3)cc2n1 ; COCc1cc(Nc2nc(N3CCNCC3)nc3cc(OCCCN4CCCC4)c(OC)cc23)n[nH]1]

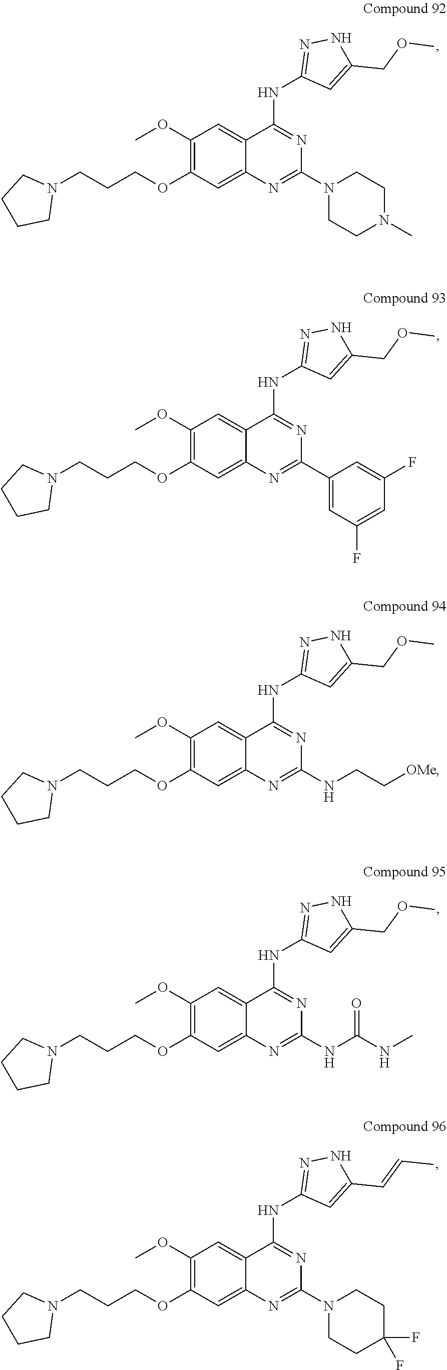 [SMILES: COCc1cc(Nc2nc(N3CCN(C)CC3)nc3cc(OCCCN4CCCC4)c(OC)cc23)n[nH]1 ; COCc1cc(Nc2nc(-c3cc(F)cc(F)c3)nc3cc(OCCCN4CCCC4)c(OC)cc23)n[nH]1 ; COCCNc1nc(Nc2cc(COC)[nH]n2)c2cc(OC)c(OCCCN3CCCC3)cc2n1 ; CNC(=O)Nc1nc(Nc2cc(COC)[nH]n2)c2cc(OC)c(OCCCN3CCCC3)cc2n1 ; C/C=C/c1cc(Nc2nc(N3CCC(F)(F)CC3)nc3cc(OCCCN4CCCC4)c(OC)cc23)n[nH]1]

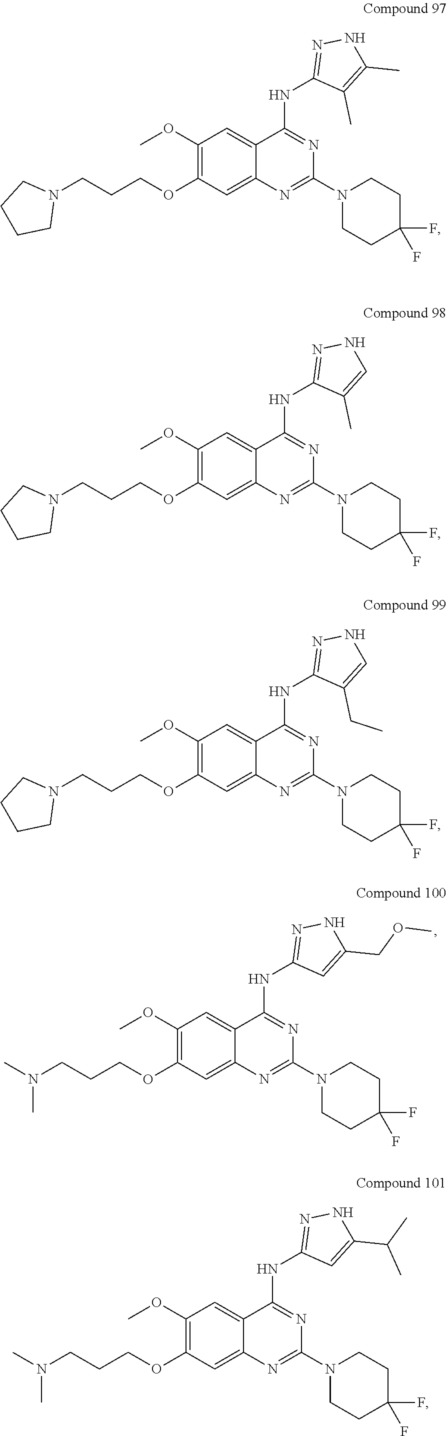

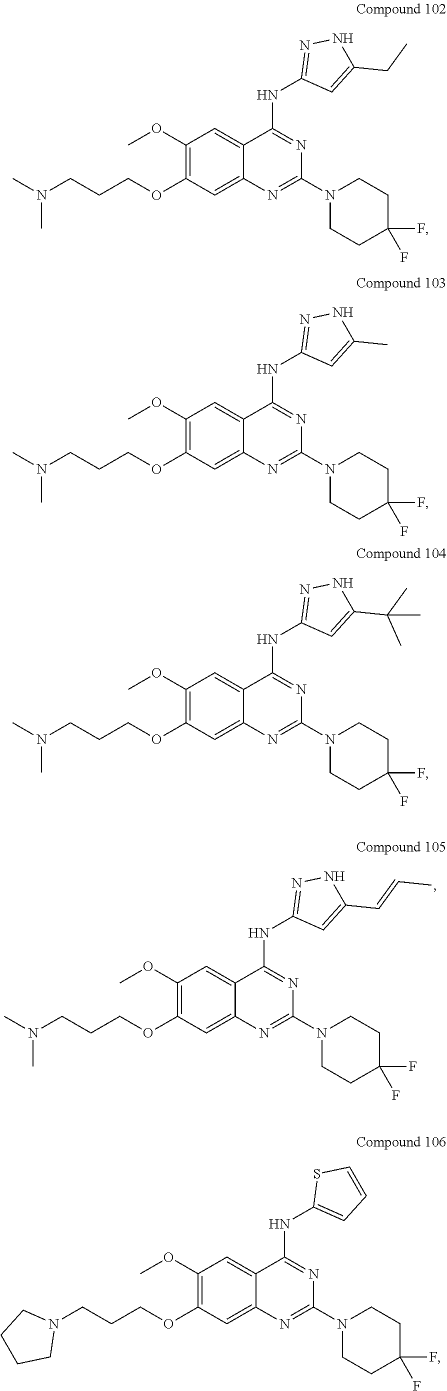 [SMILES: CCc1cc(Nc2nc(N3CCC(F)(F)CC3)nc3cc(OCCCN(C)C)c(OC)cc23)n[nH]1 ; COc1cc2c(Nc3cc(C)[nH]n3)nc(N3CCC(F)(F)CC3)nc2cc1OCCCN(C)C ; COc1cc2c(Nc3cc(C(C)(C)C)[nH]n3)nc(N3CCC(F)(F)CC3)nc2cc1OCCCN(C)C ; C/C=C/c1cc(Nc2nc(N3CCC(F)(F)CC3)nc3cc(OCCCN(C)C)c(OC)cc23)n[nH]1 ; COc1cc2c(Nc3cccs3)nc(N3CCC(F)(F)CC3)nc2cc1OCCCN1CCCC1]

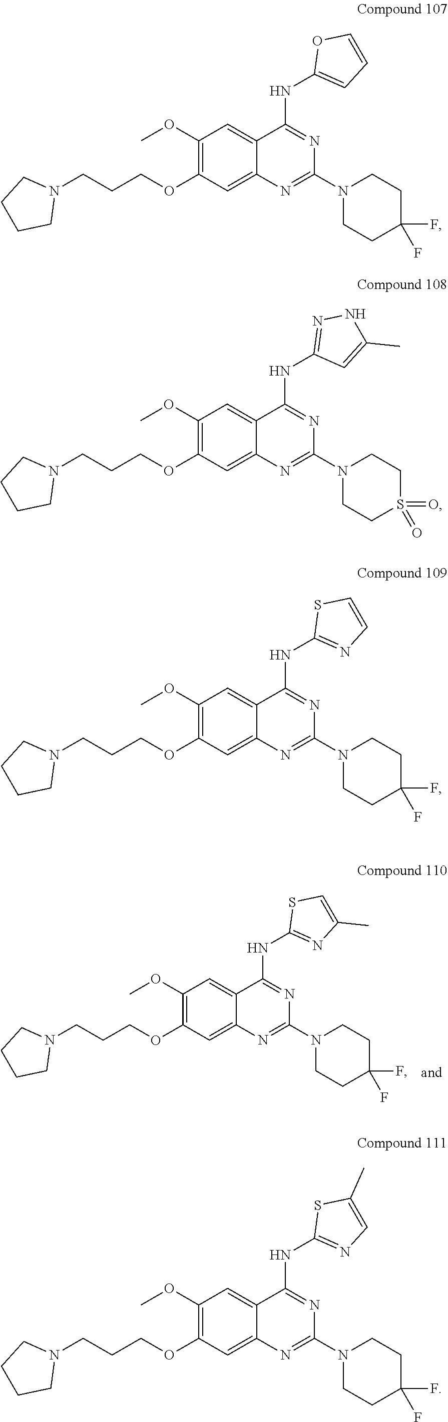 [SMILES: COc1cc2c(Nc3ccco3)nc(N3CCC(F)(F)CC3)nc2cc1OCCCN1CCCC1 ; COc1cc2c(Nc3cc(C)[nH]n3)nc(N3CCS(=O)(=O)CC3)nc2cc1OCCCN1CCCC1 ; COc1cc2c(Nc3nccs3)nc(N3CCC(F)(F)CC3)nc2cc1OCCCN1CCCC1 ; COc1cc2c(Nc3nc(C)cs3)nc(N3CCC(F)(F)CC3)nc2cc1OCCCN1CCCC1 ; COc1cc2c(Nc3ncc(C)s3)nc(N3CCC(F)(F)CC3)nc2cc1OCCCN1CCCC1]

In several embodiments, the compound of Formula (I) is represented by a compound selected from the group consisting of: 2-(4,4-difluoropiperidin-1-yl)-6-methoxy-N-(1H-pyrazol-3-yl)-7-(3-(pyrrolidin-1-yl)propoxy)quinazolin-4-amine, 2-(4,4-difluoropiperidin-1-yl)-6-methoxy-N-(5-methyl-1H-pyrazol-3-yl)-7-(3-(pyrrolidin-1-yl)propoxy)quinazolin-4-amine, N-(5-cyclopropyl-1H-pyrazol-3-yl)-2-(4,4-difluoropiperidin-1-yl)-6-methoxy-7-(3-(pyrrolidin-1-yl)propoxy)quinazolin-4-amine, N-(5-cyclobutyl-1H-pyrazol-3-yl)-2-(4,4-difluoropiperidin-1-yl)-6-methoxy-7-(3-(pyrrolidin-1-yl)propoxy)quinazolin-4-amine. 2-(4,4-difluoropiperidin-1-yl)-6-methoxy-N-(1H-pyrrol-2-yl)-7-(3-(pyrrolidin-1-yl)propoxy)quinazolin-4-amine. 2-(4,4-difluoropiperidin-1-yl)-N-(1H-imidazol-2-yl)-6-methoxy-7-(3-(pyrrolidin-1-yl)propoxy)quinazolin-4-amine, N-(2-(4,4-difluoropiperidin-1-yl)-6-methoxy-7-(3-(pyrrolidin-1-yl)propoxy)quinazolin-4-yl)oxazol-4-amine, N-(2-(4,4-difluoropiperidin-1-yl)-6-methoxy-7-(3-(pyrrolidin-1-yl)propoxy)quinazolin-4-yl)-5-methyloxazol-2-amine, N-(2-(4,4-difluoropiperidin-1-yl)-6-methoxy-7-(3-(pyrrolidin-1-yl)propoxy)quinazolin-4-yl)-5-methylthiazol-2-amine, N-(2-(4,4-difluoropiperidin-1-yl)-6-methoxy-7-(3-(pyrrolidin-1-yl)propoxy)quinazolin-4-yl)-5-methyl-1,3,4-oxadiazol-2-amine, N-(2-(4,4-difluoropiperidin-1-yl)-6-methoxy-7-(3-(pyrrolidin-1-yl)propoxy)quinazolin-4-yl)-5-methyl-1,3,4-thiadiazol-2-amine, 2-(4,4-difluoropiperidin-1-yl)-6-methoxy-N-(5-methyl-4H-1,2,4-triazol-3-yl)-7-(3-(pyrrolidin-1-yl)propoxy)quinazolin-4-amine. 2-(4,4-difluoropiperidin-1-yl)-6-methoxy-7-(3-(pyrrolidin-1-yl)propoxy)-N-(1H-tetrazol-5-yl)quinazolin-4-amine, N-(2-(4,4-difluoropiperidin-1-yl)-6-methoxy-7-(3-(pyrrolidin-1-yl)propoxy)quinazolin-4-yl)thiazol-4-amine, N-(2-(4,4-difluoropiperidin-1-yl)-6-methoxy-7-(3-(pyrrolidin-1-yl)propoxy)quinazolin-4-yl)-1,2,4-oxadiazol-3-amine, N-(2-(4,4-difluoropiperidin-1-yl)-6-methoxy-7-(3-(pyrrolidin-1-yl)propoxy)quinazolin-4-yl)-1,2,4-thiadiazol-3-amine, N-(2-(4,4-difluoropiperidin-1-yl)-6-methoxy-7-(3-(pyrrolidin-1-yl)propoxy)quinazolin-4-yl)-1,2,4-thiadiazol-3-amine, 2-(4,4-difluoropiperidin-1-yl)-6-methoxy-7-(3-(pyrrolidin-1-yl)propoxy)-N-(2H-1,2,3-triazol-4-yl)quinazolin-4-amine, 6-methoxy-2-(4-methoxypiperidin-1-yl)-N-(5-methyl-1H-pyrazol-3-yl)-7-(3-(pyrrolidin-1-yl)propoxy)quinazolin-4-amine, 6-methoxy-N2,N2-dimethyl-N4-(5-methyl-1H-pyrazol-3-yl)-7-(3-(pyrrolidin-1-yl)propoxy)quinazoline-2,4-diamine, N²-butyl-6-methoxy-N4-(5-methyl-1H-pyrazol-3-yl)-7-(3-(pyrrolidin-1-yl)propoxy)quinazoline-2,4-diamine, 6-methoxy-N-(5-methyl-1H-pyrazol-3-yl)-2-(piperazin-1-yl)-7-(3-(pyrrolidin-1-yl)propoxy)quinazolin-4-amine, 6-methoxy-N-(5-methyl-1H-pyrazol-3-yl)-2-(4-methylpiperazin-1-yl)-7-(3-(pyrrolidin-1-yl)propoxy)quinazolin-4-amine, 6-methoxy-N-(5-methyl-1H-pyrazol-3-yl)-2-morpholino-7-(3-(pyrrolidin-1-yl)propoxy)quinazolin-4-amine, N²-cyclopropyl-6-methoxy-N4-(5-methyl-1H-pyrazol-3-yl)-7-(3-(pyrrolidin-1-yl)propoxy)quinazoline-2,4-diamine, 2-(azetidin-1-yl)-6-methoxy-N-(5-methyl-1H-pyrazol-3-yl)-7-(3-(pyrrolidin-1-yl)propoxy)quinazolin-4-amine, 6-methoxy-N-(5-methyl-1H-pyrazol-3-yl)-2-(pyrrolidin-1-yl)-7-(3-(pyrrolidin-1-yl)propoxy)quinazolin-4-amine. 6-methoxy-4-((5-methyl-1H-pyrazol-3-yl)amino)-7-(3-(pyrrolidin-1-yl)propoxy)quinazoline-2-carbonitrile, 6-methoxy-N-(5-methyl-1H-pyrazol-3-yl)-2-(piperidin-1-yl)-7-(3-(pyrrolidin-1-yl)propoxy)quinazolin-4-amine, 6-methoxy-N⁴-(5-methyl-1H-pyrazol-3-yl)-7-(3-(pyrrolidin-1-yl)propoxy)quinazoline-2,4-diamine, 2-(3,5-difluorophenyl)-6-methoxy-N-(5-methyl-1H-pyrazol-3-yl)-7-(3-(pyrrolidin-1-yl)propoxy)quinazolin-4-amine, 6-methoxy-N²-(2-methoxyethyl)-N⁴-(5-methyl-1H-pyrazol-3-yl)-7-(3-(pyrrolidin-1-yl)propoxy)quinazoline-2,4-diamine, 1-(6-methoxy-4-((5-methyl-1H-pyrazol-3-yl)amino)-7-(3-(pyrrolidin-1-yl)propoxy)quinazolin-2-yl)-3-methylurea. 2-(4,4-difluoropiperidin-1-yl)-6-methoxy-N-(5-methyl-1H-pyrazol-3-yl)-7-(3-(pyrrolidin-1-yl)prop-1-yn-1-yl)quinazolin-4-amine, N-(2-(4,4-difluoropiperidin-1-yl)-6-methoxy-7-(3-(pyrrolidin-1-yl)prop-1-yn-1-yl)quinazolin-4-yl)-5-methylisoxazol-3-amine, N-(2-(4,4-difluoropiperidin-1-yl)-6-methoxy-7-(3-(pyrrolidin-1-yl)prop-1-yn-1-yl)quinazolin-4-yl)-5-methylthiazol-2-amine, N-(2-(4,4-difluoropiperidin-1-yl)-6-methoxy-7-(3-(pyrrolidin-1-yl)prop-1-yn-1-yl)quinazolin-4-yl)-5-methyloxazol-2-amine, N-(2-(4,4-difluoropiperidin-1-yl)-6-methoxy-7-(3-(pyrrolidin-1-yl)prop-1-yn-1-yl)quinazolin-4-yl)-5-methyl-1,3,4-oxadiazol-2-amine, 2-(4,4-difluoropiperidin-1-yl)-6-methoxy-N-(5-methyl-1H-imidazol-2-yl)-7-(3-(pyrrolidin-1-yl)prop-1-yn-1-yl)quinazolin-4-amine, 1-(6-methoxy-4-((5-methyl-1H-pyrazol-3-yl)amino)-7-(3-(pyrrolidin-1-yl)propoxy)quinazolin-2-yl)piperidin-4-ol, N⁴-(5-ethyl-1H-pyrazol-3-yl)-6-methoxy-N²,N²-dimethyl-7-(3-(pyrrolidine-1-yl)propoxy)quinazoline-2,4-diamine, N²-butyl-N⁴-(5-ethyl-1H-pyrazol-3-yl)-6-methoxy-7-(3-(pyrrolidin-1-yl)propoxy)quinazoline-2,4-diamine, N-(5-ethyl-1H-pyrazol-3-yl)-6-methoxy-2-(piperazin-1-yl)-7-(3-(pyrrolidin-1-yl)propoxy)quinazolin-4-amine, N-(5-ethyl-1H-pyrazol-3-yl)-6-methoxy-2-(4-methylpiperazin-1-yl)-7-(3-(pyrrolidin-1-yl)propoxy)quinazolin-4-amine, N-(5-ethyl-1H-pyrazol-3-yl)-6-methoxy-2-morpholino-7-(3-(pyrrolidin-1-yl)propoxy)quinazolin-4-amine, N²-cyclopropyl-N⁴-(5-ethyl-1H-pyrazol-3-yl)-6-methoxy-7-(3-(pyrrolidine-1-yl)propoxy)quinazoline-2,4-diamine, 2-(azetidin-1-yl)-N-(5-ethyl-1H-pyrazol-3-yl)-6-methoxy-7-(3-(pyrrolidin-1-yl)propoxy)quinazolin-4-amine, N-(5-ethyl-1H-pyrazol-3-yl)-6-methoxy-2-(pyrrolidin-1-yl)-7-(3-(pyrrolidin-1-yl)propoxy)quinazolin-4-amine, 4-((5-ethyl-1H-pyrazol-3-yl)amino)-6-methoxy-7-(3-(pyrrolidin-1-yl)propoxy)quinazoline-2-carbonitrile, N-(5-ethyl-1H-pyrazol-3-yl)-6-methoxy-2-(piperidin-1-yl)-7-(3-(pyrrolidin-1-yl)propoxy)quinazolin-4-amine, N⁴-(5-ethyl-1H-pyrazol-3-yl)-6-methoxy-7-(3-(pyrrolidin-1-yl)propoxy)quinazoline-2,4-diamine, 2-(3,5-difluorophenyl)-N-(5-ethyl-1H-pyrazol-3-yl)-6-methoxy-7-(3-(pyrrolidin-1-yl)propoxy)quinazolin-4-amine, N⁴-(5-ethyl-1H-pyrazol-3-yl)-6-methoxy-N²-(2-methoxyethyl)-7-(3-(pyrrolidine-1-yl)propoxy)quinazoline-2,4-diamine, 1-(4-((5-ethyl-1H-pyrazol-3-yl)amino)-6-methoxy-7-(3-(pyrrolidin-1-yl)propoxy)quinazolin-2-yl)-3-methylurea, N-(5-ethyl-1H-pyrazol-3-yl)-6-methoxy-2-(4-methoxypiperidin-1-yl)-7-(3-(pyrrolidin-1-yl)propoxy)quinazolin-4-amine, N⁴-(5-cyclopropyl-1H-pyrazol-3-yl)-6-methoxy-N²,N²-dimethyl-7-(3-(pyrrolidin-1-yl)propoxy)quinazoline-2,4-diamine, N²-butyl-N⁴-(5-cyclopropyl-1H-pyrazol-3-yl)-6-methoxy-7-(3-(pyrrolidin-1-yl)propoxy)quinazoline-2,4-diamine, N-(5-cyclopropyl-1H-pyrazol-3-yl)-6-methoxy-2-(piperazin-1-yl)-7-(3-(pyrrolidin-1-yl)propoxy)quinazolin-4-amine, N-(5-cyclopropyl-1H-pyrazol-3-yl)-6-methoxy-2-(4-methylpiperazin-1-yl)-7-(3-(pyrrolidin-1-yl)propoxy)quinazolin-4-amine, N-(5-cyclopropyl-1H-pyrazol-3-yl)-6-methoxy-2-morpholino-7-(3-(pyrrolidin-1-yl)propoxy)quinazolin-4-amine, N²-cyclopropyl-N⁴-(5-cyclopropyl-1H-pyrazol-3-yl)-6-methoxy-7-(3-(pyrrolidin-1-yl)propoxy)quinazoline-2,4-diamine, 2-(azetidin-1-yl)-N-(5-cyclopropyl-1H-pyrazol-3-yl)-6-methoxy-7-(3-(pyrrolidin-1-yl)propoxy)quinazolin-4-amine, N-(5-cyclopropyl-1H-pyrazol-3-yl)-6-methoxy-2-(pyrrolidin-1-yl)-7-(3-(pyrrolidin-1-yl)propoxy)quinazolin-4-amine, 4-((5-cyclopropyl-1H-pyrazol-3-yl)amino)-6-methoxy-7-(3-(pyrrolidin-1-yl)propoxy)quinazoline-2-carbonitrile, N-(5-cyclopropyl-1H-pyrazol-3-yl)-6-methoxy-2-(piperidin-1-yl)-7-(3-(pyrrolidin-1-yl)propoxy)quinazolin-4-amine, N⁴-(5-cyclopropyl-1H-pyrazol-3-yl)-6-methoxy-7-(3-(pyrrolidin-1-yl)propoxy)quinazoline-2,4-diamine, N-(5-cyclopropyl-1H-pyrazol-3-yl)-2-(3,5-difluorophenyl)-6-methoxy-7-(3-(pyrrolidin-1-yl)propoxy)quinazolin-4-amine, N⁴-(5-cyclopropyl-1H-pyrazol-3-yl)-6-methoxy-N²-(2-methoxyethyl)-7-(3-(pyrrolidin-1-yl)propoxy)quinazoline-2,4-diamine, 1-(4-((5-cyclopropyl-1H-pyrazol-3-yl)amino)-6-methoxy-7-(3-(pyrrolidin-1-yl)propoxy)quinazolin-2-yl)-3-methylurea, N-(5-cyclopropyl-1H-pyrazol-3-yl)-6-methoxy-2-(4-methoxypiperidin-1-yl)-7-(3-(pyrrolidin-1-yl)propoxy)quinazolin-4-amine, 6-methoxy-N⁴-(5-(methoxymethyl)-1H-pyrazol-3-yl)-N2,N2-dimethyl-7-(3-(pyrrolidin-1-yl)propoxy)quinazoline-2,4-diamine, N²-butyl-6-methoxy-N⁴-(5-(methoxymethyl)-1H-pyrazol-3-yl)-7-(3-(pyrrolidine-1-yl)propoxy)quinazoline-2,4-diamine, 6-methoxy-N-(5-(methoxymethyl)-1H-pyrazol-3-yl)-2-(piperazin-1-yl)-7-(3-(pyrrolidin-1-yl)propoxy)quinazolin-4-amine, 6-methoxy-N-(5-(methoxymethyl)-1H-pyrazol-3-yl)-2-(4-methylpiperazin-1-yl)-7-(3-(pyrrolidin-1-yl)propoxy)quinazolin-4-amine, 6-methoxy-N-(5-(methoxymethyl)-1H-pyrazol-3-yl)-2-morpholino-7-(3-(pyrrolidin-1-yl)propoxy)quinazolin-4-amine, 2-(3,5-difluorophenyl)-6-methoxy-N-(5-(methoxymethyl)-1H-pyrazol-3-yl)-7-(3-6-methoxy-N2-(2-methoxyethyl)-N⁴-(5-(pyrrolidin-1-yl)propoxy)quinazolin-4-amine, (methoxymethyl)-1H-pyrazol-3-yl)-7-(3-(pyrrolidin-1-yl)propoxy)quinazoline-2,4-diamine, 1-(6-methoxy-4-((5-(methoxymethyl)-1H-pyrazol-3-yl)amino)-7-(3-(pyrrolidin-1-yl)propoxy)quinazolin-2-yl)-3-methylurea, 6-methoxy-N-(5-(methoxymethyl)-1H-pyrazol-3-yl)-2-(4-methoxypiperidin-1-yl)-7-(3-(pyrrolidin-1-yl)propoxy)quinazolin-4-amine, 2-(4,4-difluoropiperidin-1-yl)-6-methoxy-N-(5-(methoxymethyl)-1H-pyrazol-3-yl)-7-(3-(pyrrolidin-1-yl)propoxy)quinazolin-4-amine. (E)-2-(4,4-difluoropiperidin-1-yl)-6-methoxy-N-(5-(prop-1-en-1-yl)-1H-pyrazol-3-yl)-7-(3-(pyrrolidin-1-yl)propoxy)quinazolin-4-amine, 2-(4,4-difluoropiperidin-1-yl)-N-(4,5-dimethyl-1H-pyrazol-3-yl)-6-methoxy-7-(3-(pyrrolidin-1-yl)propoxy)quinazolin-4-amine, 2-(4,4-difluoropiperidin-1-yl)-6-methoxy-N-(4-methyl-1H-pyrazol-3-yl)-7-(3-(pyrrolidin-1-yl)propoxy)quinazolin-4-amine, 2-(4,4-difluoropiperidin-1-yl)-N-(4-ethyl-1H-pyrazol-3-yl)-6-methoxy-7-(3-(pyrrolidin-1-yl)propoxy)quinazolin-4-amine, 2-(4,4-difluoropiperidin-1-yl)-7-(3-(dimethylamino)propoxy)-6-methoxy-N-(5-(methoxymethyl)-1H-pyrazol-3-yl)quinazolin-4-amine, 2-(4,4-difluoropiperidin-1-yl)-7-(3-(dimethylamino)propoxy)-N-(5-isopropyl-1H-pyrazol-3-yl)-6-methoxyquinazolin-4-amine, 2-(4,4-difluoropiperidin-1-yl)-7-(3-(dimethylamino)propoxy)-N-(5-ethyl-1H-pyrazol-3-yl)-6-methoxyquinazolin-4-amine, 2-(4,4-difluoropiperidin-1-yl)-7-(3-(dimethylamino)propoxy)-6-methoxy-N-(5-methyl-1H-pyrazol-3-yl)quinazolin-4-amine, N-(5-(tert-butyl)-1H-pyrazol-3-yl)-2-(4,4-difluoropiperidin-1-yl)-7-(3-(dimethylamino)propoxy)-6-methoxyquinazolin-4-amine, (E)-2-(4,4-difluoropiperidin-1-yl)-7-(3-(dimethylamino)propoxy)-6-methoxy-N-(5-(prop-1-en-1-yl)-1H-pyrazol-3-yl)quinazolin-4-amine, N-(5-(tert-butyl)-1H-pyrazol-3-yl)-2-(4,4-difluoropiperidin-1-yl)-6-methoxy-7-(3-(pyrrolidin-1-yl)propoxy)quinazolin-4-amine, 2-(4,4-difluoropiperidin-1-yl)-N-(5-ethyl-1H-pyrazol-3-yl)-6-methoxy-7-(3-(pyrrolidin-1-yl)propoxy)quinazolin-4-amine, 2-(4,4-difluoropiperidin-1-yl)-N-(5-isopropyl-1H-pyrazol-3-yl)-6-methoxy-7-(3-(pyrrolidin-1-yl)propoxy)quinazolin-4-amine, 2-(4,4-difluoropiperidin-1-yl)-6-methoxy-N-methyl-N-(5-methyl-1H-pyrazol-3-yl)-7-(3-(pyrrolidin-1-yl)propoxy)quinazolin-4-amine, 6-methoxy-N-(5-(methoxymethyl)-1H-pyrazol-3-yl)-2-(pyrrolidin-1-yl)-7-(3-(pyrrolidin-1-yl)propoxy)quinazolin-4-amine, N-(2-(4,4-difluoropiperidin-1-yl)-6-methoxy-7-(3-(pyrrolidin-1-yl)propoxy)quinazolin-4-yl)isoxazol-3-amine, 2-(4,4-difluoropiperidin-1-yl)-6-methoxy-N-(1H-pyrrol-3-yl)-7-(3-(pyrrolidin-1-yl)propoxy)quinazolin-4-amine, 2-(4,4-difluoropiperidin-1-yl)-6-methoxy-7-(3-(pyrrolidin-1-yl)propoxy)-N-(thiophen-2-yl)quinazolin-4-amine, 2-(4,4-difluoropiperidin-1-yl)-N-(furan-2-yl)-6-methoxy-7-(3-(pyrrolidin-1-yl)propoxy)quinazolin-4-amine, and 4-(6-methoxy-4-(methyl(5-methyl-1H-pyrazol-3-yl)amino)-7-(3-(pyrrolidin-1-yl)propoxy)quinazolin-2-yl)thiomorpholine 1,1-dioxide.

In several embodiments, optionally substituted groups (e.g., of Formula (I), etc.) may be substituted with one or more substituent(s) independently selected from C₁-C₄ alkyl, C₁-C₄ alkoxy, amino, hydroxy, and halogen. In several embodiments, when substituted, the optional substitutions of the R¹ are selected from one or more of amino, —OH, optionally substituted C₁-C₆ alkyl, optionally substituted C₁-C₆ alkoxy, and halogen. In several embodiments, when substituted, the optional substitutions of the R² are selected from one or more of amino, —OH, optionally substituted C₁-C₆ alkyl, optionally substituted C₁-C₆ alkoxy, and halogen. In several embodiments, when substituted, the optional substitutions of the R^{a} are selected from one or more of amino, —OH, optionally substituted C₁-C₆ alkyl, optionally substituted C₁-C₆ alkoxy, and halogen.

### Compounds of Formula (IA)

In several embodiments, the compound of Formula (I) is further represented by the structure of Formula (IA):

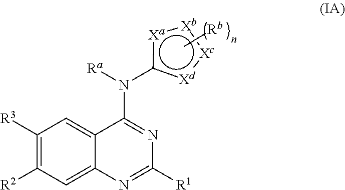

where the A ring is represented by ring structure (AIa); and where the remaining variables are as defined elsewhere herein. In several embodiments, the ring structure (AIa) is further represented by one of the following structures:

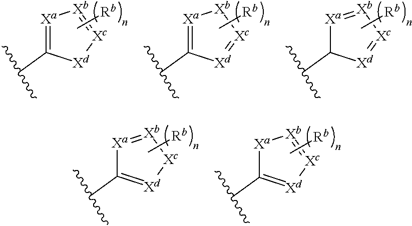

### Compounds of Formula (IB)

In several embodiments, the compound of Formula (I) is further represented by the structure of Formula (IB):

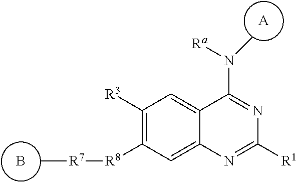

where the B ring is represented heteroaryl; R⁷ is selected from the group consisting of C₁-C₆ alkyl and C₁-C₆ alkynyl; R⁸ is O or is absent (e.g., a direct bond between the quinazolinyl ring and R⁷); and the remaining variables are as defined elsewhere herein. In several embodiments, the B ring is represented by:

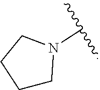

In several embodiments, the R⁷ together with R⁸, where present, is a structure selected from the group consisting of:

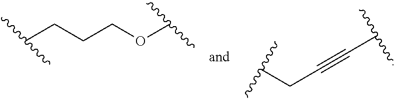

### Compounds of Formula (IC)

In several embodiments, the compound of Formula (I) is further represented by the structure of Formula (IC):

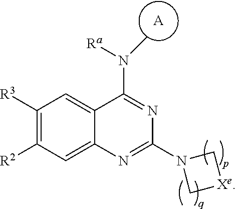

where p and q are integers selected from 1, 2, or 3; X^{e} is selected from the group consisting of C(R^{d})₂, N(R^{d}), O, S, and S(O)₂; each instance of R^{d}, where present, is independently selected from the group consisting of —H, halogen, and C₁-C₆ alkyl. In several embodiments, each instance of R^{d}, where present, is independently selected from the group consisting of —H, —F, and -Me.

In several embodiments, where the variables of one formula are not defined (e.g., any one of Formulae (IA), (IB), (IC), (II), (IIp), etc.), those variables may be defined as provided anywhere else herein (e.g., as for Formula (I), etc.).

### Methods of Treating

Several embodiments relate to treating a disorder, comprising administering to a subject in need thereof a quinazolinyl compound or pharmaceutical composition comprising a quinazolinyl compound as described herein. In several embodiments, the disorder is related to a kinase enzyme.

Cancer results from the regulation of the normal processes that control cell division, differentiation, and apoptotic cell death such that protein kinases play a critical role in this regulatory process. Therefore, a partial non-limiting list of such kinases that the disclosed quinazolinyl compounds target directly includes: CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, LTK. Other cancer related kinases targeted by the disclosed quinazolinyl include (without limitation): abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, and Zap70.

In mammalian biology, such protein kinases comprise mitogen activated protein kinase (MAPK) signaling pathway. In several embodiments, the disclosed quinazolinyl compounds are used in methods of disrupting the MAPK signaling pathway (e.g., to treat cancer, autoimmune disease, and/or DMD).

Lipid kinases (e.g. PI3K) constitute a separate group of kinases with structural similarity to protein kinases. In several embodiments, the disclosed quinazolinyl compounds may be used in methods of treating lipid kinase mediated disorders (e.g., cancer, autoimmune disease, and/or DMD).

Several embodiments relate to a method of treating a kinase (e.g., protein or lipid) related disease or disorder (e.g., cancer, autoimmune disease, and/or DMD), comprising administering to a subject in need thereof a quinazolinyl compound as described herein, or a pharmaceutical composition as described herein. In several embodiments, the kinase-related disease is cancer, autoimmune disease, and/or DMD. In several embodiments, the cancer is selected from the group consisting of colorectal, gastric, stomach, esophageal, liver, pancreatic, breast, prostate, bladder, renal, ovarian, lung, melanoma, and multiple myeloma. In several embodiments, the autoimmune disease is selected from the group consisting of Ulcerative Colitis, Crohn's disease, systemic lupus erythematosus, psoriasis, rheumatoid arthritis, type 1 diabetes, multiple sclerosis, celiac disease, Graft versus host disease (GVHD), Sjogren syndrome, Graves' Disease, Hashimoto's Thyroiditis, Autoimmune Hepatitis, Behcet's Disease, atopic dermatitis, Castleman disease, Allergic Rhinitis, Eczema, Dressler's Syndrome, Eosinophilic esophagitis, Fibromyalgia, Guillain-Barre Syndrome, Juvenile arthritis, Kawasaki disease, Mooren's ulcer, mixed connective tissue disease, Parry Romberg syndrome, primary biliary cirrhosis, primary sclerosing cholangitis, psoriatic arthritis, sarcoidosis, scleroderma, undifferentiated connective tissue disease, uveitis, vasculitis and vitiligo. In several embodiments, the method of treating cancer includes administering one or more compounds of Formula (I) to a patient who is suspected of having a cancer or being at risk of having a cancer. In several embodiments, the method of treating cancer, autoimmune disease, and/or DMD includes administering one or more compounds of Formula (I) to a patient who has cancer, autoimmune disease, and/or DMD.

In several embodiments, the kinase inhibited by the quinazolinyl compound is selected from the group consisting of CLK1, CLK4, PLK4, FLT3, and JNK1. It has been found that all these kinases have cancer disease relevance, JNK1 has relevance to autoimmune diseases, and CLK1/4 are related to Duchenne muscular dystrophy (DMD).

In several embodiments, the compounds as disclosed herein are characterized by their ability to bind one or more of kinases as disclosed herein. In several embodiments, the compounds of Formula (I) are characterized by a dissociation constant (Kd) for a kinase as disclosed herein of equal to or less than about: 1200 nM, 1000 nM, 780 nM, 500 nM, 250 nM, 150 nM, 100 nM, 50 nM, 25 nM, 10 nM, 5 nM, 1 nM, 0.1 nM, 0.01 nM, or ranges including and/or spanning the aforementioned values. In several embodiments, Kd values may be measured in aqueous 0.9% DMSO solution. In several embodiments, Kd is measured using liganded affinity beads as disclosed elsewhere herein. In several embodiments, Kd is measured by incubating combining kinases, liganded affinity beads, and compounds in 1× binding buffer (e.g., 20% ScaBlock, 0.17×PBS, 0.05% Tween 20, 6 mM DTT).

In several embodiments, the compounds as disclosed herein are characterized by their ability to bind one or more of CLK1, CLK4, PLK4, FLT3, and/or JNK1 as disclosed herein. In several embodiments, the compounds of Formula (I) are characterized by a dissociation constant (Kd) for CLK1, CLK4, PLK4, FLT3, and/or JNK1 of equal to or less than about: 1200 nM, 1000 nM, 780 nM, 500 nM, 250 nM, 150 nM, 100 nM, 50 nM, 25 nM, 10 nM, 5 nM, 1 nM, 0.1 nM, 0.01 nM, or ranges including and/or spanning the aforementioned values. In several embodiments, the compounds as disclosed herein are characterized by their ability to bind one or more of CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and/or LTK as disclosed herein. In several embodiments, the compounds of Formula (I) are characterized by a dissociation constant (Kd) for CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and/or LTK of equal to or less than about: 1200 nM, 1000 nM, 780 nM, 500 nM, 250 nM, 150 nM, 100 nM, 50 nM, 25 nM, 10 nM, 5 nM, 1 nM, 0.1 nM, 0.01 nM, or ranges including and/or spanning the aforementioned values. In several embodiments, the compounds as disclosed herein are characterized by their ability to bind one or more of abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, and/or Zap70 as disclosed herein. In several embodiments, the compounds of Formula (I) are characterized by a dissociation constant (Kd) for abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, and/or Zap70 of equal to or less than about: 1200 nM, 1000 nM, 780 nM, 500 nM, 250 nM, 150 nM, 100 nM, 50 nM, 25 nM, 10 nM, 5 nM, 1 nM, 0.1 nM, 0.01 nM, or ranges including and/or spanning the aforementioned values. In several embodiments, Kd values may be measured in aqueous 0.9% DMSO solution. In several embodiments, Kd is measured using liganded affinity beads as disclosed elsewhere herein. In several embodiments, Kd is measured by incubating combining kinases, liganded affinity beads, and compounds in 1× binding buffer (e.g., 20% SeaBlock, 0.17×PBS, 0.05% Tween 20, 6 mM DTT).

In several embodiments, the kinase enzyme target (e.g., for inhibition) is a mutant enzyme and not the wild-type enzyme. In several embodiments, the inhibitor can be a compound of Formula (I). In several embodiments, a compound of Formula (I) is at least 1.1, 2, 5, 10, 20, 30, 40, 50, 60, 70, 80, 90, or 100 fold as potent for the mutant as it is for wild-type. In several embodiments, the IC₅₀ for a compound of Formula (I) is 0.5, 0.1, 0.05, or 0.01% as large for the mutant as it is for wild type (that is, the numerical value for the IC₅₀ is lower for the mutant). In several embodiments, the IC₅₀ of a compound of Formula (I) to the mutant or mutation is no higher than about 100 nM (e.g., it is at least as good in potency as 100 nM). In several embodiments, the IC₅₀ of a compound of Formula (I) to the mutant or mutation is no higher than about 10 nM (e.g., it is at least as good in potency as 10 nM). In several embodiments, the IC₅₀ of a compound of Formula (I) to the mutant or mutation is no higher than single digit nM (e.g., it is at least as good in potency as single digit nM). In several embodiments, the IC₅₀ of a compound of Formula (I) to the mutant or mutation is at least as effective for the mutant or mutation as it is for a wild type kinase.

In several embodiments, the kinase enzyme target (e.g., for inhibition) is a mutant CLK1, CLK4, PLK4, FLT3, and/or JNK1 enzyme and not the wild-type enzyme. In several embodiments, the inhibitor can be a compound of Formula (I). In several embodiments, a compound of Formula (I) is at least 1.1, 2, 5, 10, 20, 30, 40, 50, 60, 70, 80, 90, or 100 fold as potent for the mutant as it is for wild-type. In several embodiments, the IC₅₀ for a compound of Formula (I) is 0.5, 0.1, 0.05, or 0.01% as large for the mutant CLK1, CLK4, PLK4, FLT3, and/or JNK1 as it is for wild type (that is, the numerical value for the IC₅₀ is lower for the mutant). In several embodiments, the IC₅₀ of a compound of Formula (I) to the mutant or mutation of CLK1, CLK4, PLK4, FLT3, and/or JNK1 is no higher than about 100 nM (e.g., it is at least as good in potency as 100 nM). In several embodiments, the IC₅₀ of a compound of Formula (I) to the mutant or mutation of CLK1, CLK4, PLK4, FLT3, and/or JNK1 is no higher than about 10 nM (e.g., it is at least as good in potency as 10 nM). In several embodiments, the IC₅₀ of a compound of Formula (I) to the mutant or mutation of CLK1, CLK4, PLK4, FLT3, and/or JNK1 is no higher than single digit nM (e.g., it is at least as good in potency as single digit nM). In several embodiments, the IC₅₀ of a compound of Formula (I) to the mutant or mutation of CLK1, CLK4, PLK4, FLT3, and/or JNK1 is at least as effective for the mutant or mutation as it is for a wild type kinase.

In several embodiments, the kinase enzyme target (e.g., for inhibition) is a mutant CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and/or LTK enzyme and not the wild-type enzyme. In several embodiments, the inhibitor can be a compound of Formula (I). In several embodiments, a compound of Formula (I) is at least 1.1, 2, 5, 10, 20, 30, 40, 50, 60, 70, 80, 90, or 100 fold as potent for the mutant as it is for wild-type. In several embodiments, the IC₅₀ for a compound of Formula (I) is 0.5, 0.1, 0.05, or 0.01% as large for the mutant CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and/or LTK as it is for wild type (that is, the numerical value for the IC₅₀ is lower for the mutant). In several embodiments, the IC₅₀ of a compound of Formula (I) to the mutant or mutation of CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and/or LTK is no higher than about 100 nM (e.g., it is at least as good in potency as 100 nM). In several embodiments, the IC₅₀ of a compound of Formula (I) to the mutant or mutation of CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and/or LTK is no higher than about 10 nM (e.g., it is at least as good in potency as 10 nM). In several embodiments, the IC₅₀ of a compound of Formula (I) to the mutant or mutation of CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and/or LTK is no higher than single digit nM (e.g., it is at least as good in potency as single digit nM). In several embodiments, the IC₅₀ of a compound of Formula (I) to the mutant or mutation of CLK1, CLK2, CLK3, CLK4, FMS, JNK1, JNK2, JNK3, PLK4, FLT3, FLT3 (D835V), FLT3 (ITD), FLT3 (F691L), FLT3 (N841I), FLT3 (D835H), FLT3 (D835Y), FLT3 (K663Q), FLT3 (N8411), MYLK4, NUAK2, CSFIR. DAPK3, RIOK2, HIPK1, ALK, MYLK, EGFR, FGFR1, FGFR2, FGFR3, FGFR4, FGFR5, VEGFR, JAK1, ABL1, DAPK2, and/or LTK is at least as effective for the mutant or mutation as it is for a wild type kinase.

In several embodiments, the kinase enzyme target (e.g., for inhibition) is a mutant abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR. Lck, Lyn, MEK, p38, PDGFR, PIK, PKC, PYK2, Ros, Tie1, Tie2, Trk, Yes, and/or Zap70 enzyme and not the wild-type enzyme. In several embodiments, the inhibitor can be a compound of Formula (I). In several embodiments, a compound of Formula (I) is at least 1.1, 2, 5, 10, 20, 30, 40, 50, 60, 70, 80, 90, or 100 fold as potent for the mutant as it is for wild-type. In several embodiments, the IC₅₀ for a compound of Formula (I) is 0.5, 0.1, 0.05, or 0.01% as large for the mutant abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, and/or Zap70 as it is for wild type (that is, the numerical value for the IC₅₀ is lower for the mutant). In several embodiments, the IC₅₀ of a compound of Formula (I) to the mutant or mutation of abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, and/or Zap70 is no higher than about 100 nM (e.g., it is at least as good in potency as 100 nM). In several embodiments, the IC₅₀ of a compound of Formula (I) to the mutant or mutation of abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck. Lyn, MEK, p38, PDGFR, PIK, PKC, PYK2, Ros, Tie1, Tie2, Trk, Yes, and/or Zap70 is no higher than about 10 nM (e.g., it is at least as good in potency as 10 nM). In several embodiments, the IC₅₀ of a compound of Formula (I) to the mutant or mutation of abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, and/or Zap70 is no higher than single digit nM (e.g., it is at least as good in potency as single digit nM). In several embodiments, the IC₅₀ of a compound of Formula (I) to the mutant or mutation of abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR. Lck, Lyn, MEK, p38, PDGFR, PIK, PKC, PYK2, Ros, Tie1, Tie2, Trk, Yes, and/or Zap70 is at least as effective for the mutant or mutation as it is for a wild type kinase.

In several embodiments of the method, the subject has been (or is still) on a multi-targeted kinase inhibitor (“MKI”) or a targeted kinase inhibitor. While on the MKI or the targeted inhibitor, the subject develops a tumor that has become resistant to the prior MKI or the targeted inhibitor. At this point, one can either simply administer a compound of Formula (I). In the alternative, one can determine if the subject now has a tumor that has a kinase mutation in it (such as amino acid changes that result in resistance to the prior therapy). If the subject does have a tumor with the noted mutation, one can then dose the subject with a compound of Formula (I).

### Administration and Pharmaceutical Compositions

In several embodiments, the quinazolinyl compounds are administered at a therapeutically effective dosage. In several embodiments, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the quinazolinyl compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarily, vaginally, rectally, or intraocularly. Oral and parenteral administrations are customary in treating the indications that are the subject of the preferred embodiments.

The compounds quinazolinyl useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject quinazolinyl compound is basically determined by the way the compound is to be administered.

The quinazolinyl compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The quinazolinyl compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

In several embodiments, the oral comprises formulation dimethylacetamide (DMA). In several embodiments, the oral formulation comprises DMA in an amount (in wt %) of equal or less than about: 1%, 5%, 7.5%, 10%, 15%, or ranges including and/or spanning the aforementioned values. In several embodiments, the oral formulation comprises propylene glycol (PG). In several embodiments, the oral formulation comprises PG in an amount (in wt %) of equal or less than about: 10%, 20%, 25%, 30%, 35%, or ranges including and/or spanning the aforementioned values. In several embodiments, the oral formulation comprises polyethylene glycol (PEG). In several embodiments, the oral formulation comprises PEG in an amount (in wt %) of equal or less than about: 15%, 25%, 30%, 35%, 40%, or ranges including and/or spanning the aforementioned values. In several embodiments, the oral formulation comprises water. In several embodiments, the oral formulation comprises water in an amount (in wt %) of equal or less than about: 15%, 25%, 30%, 35%, 40%, or ranges including and/or spanning the aforementioned values.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives (e.g., active pharmaceutical agents). In some embodiments, the composition may comprise one or more quinazolinyl compounds as disclosed elsewhere herein.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The quinazolinyl compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 to 99.99 wt % of a compound of the present technology based on the total formulation, with the balance being one or more suitable pharmaceutical excipients. Preferably, the compound is present at a level of about 1 to 80 wt %. Representative pharmaceutical formulations are described below.

In several embodiments, oral formulations of the compounds described herein may be formulated readily by combining the active compounds with pharmaceutically acceptable carriers and excipients. Such carriers enable the compounds of the present disclosure to be formulated as tablets, pills, capsules, liquids, gels, syrups, slurries, suspensions, emulsions, and the like, for oral ingestion by a subject. Pharmacological preparations for oral use may be made using a solid excipient, optionally grinding the resulting mixture, and processing the mixture of granules, after adding suitable auxiliaries if desired, to obtain tablets. Suitable excipients are, in particular, fillers such as sugars, including lactose, sucrose, mannitol, or sorbitol; cellulose preparations such as, for example, maize starch, wheat starch, rice starch, potato starch, gelatin, methyl cellulose, hydroxypropylmethyl-cellulose and sodium carboxymethylcellulose.

In several embodiments, pharmaceutical compositions of the compounds described herein that may be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally stabilizers.

Several embodiments comprise the compounds described herein encapsulated in soft capsules, in which the active compounds may be dissolved or suspended in suitable liquids, such as fatty oils, liquid paraffin, or liquid polyethylene glycols. In addition, stabilizers could be added.

In several embodiments, the dosage of a composition to be administered would depend on several factors including the subject being treated, the stage of the autoimmune disease, the route of administration, and the judgment of the prescribing clinician.

### Methods of Preparation

The compounds disclosed herein may be synthesized by methods described below, or by modification of these methods. In several embodiments, the method of synthesizing a quinazolinyl compound comprises obtaining a quinazolinyl precursor (e.g., Formula (IIp) and reacting it with an amine-containing group.

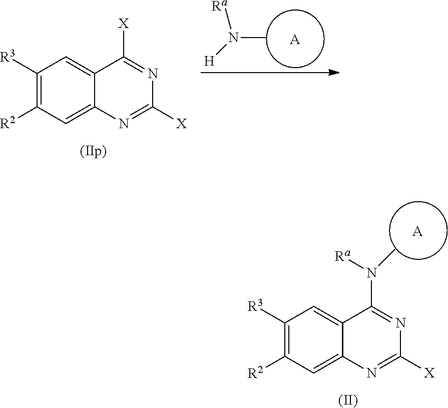

In several embodiments, X is a halogen atom (e.g., F, Cl, I, or Br) and the remaining variables are as defined elsewhere herein. In several embodiments, X is Cl. In several embodiments, R² is-OR⁶ or optionally substituted (heterocyclyl)alkynyl. In several embodiments, R⁶ is selected from the group consisting of methyl, optionally substituted 2-10 membered heteroalkyl, and (heterocyclyl)alkyl. In several embodiments, R³ is selected from the group consisting of hydrogen, halogen, and —OMe. In several embodiments, R^{a} is hydrogen or optionally substituted C₁-C₁₀ alkyl. In several embodiments, the A ring is an optionally substituted heteroaryl.

In several embodiments, the method of synthesizing a quinazolinyl compound comprises obtaining a quinazolinyl precursor (e.g., Formula (II) and reacting it with a nucleophilic group having the formula H—R¹.

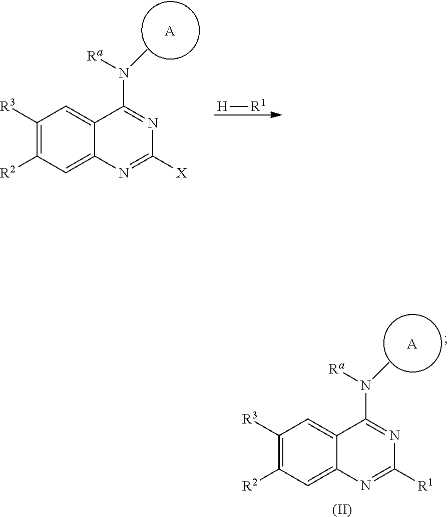

In several embodiments, X is a halogen atom (e.g., F, Cl, I, or Br) and the remaining variables are as defined elsewhere herein. In several embodiments, R¹ is selected from the group consisting of optionally substituted 6-10 membered aryl, optionally substituted 3-10 membered heterocyclyl, optionally substituted 5-10 membered heteroaryl, optionally substituted carbamide, —CN, and —NR⁴R⁵. In several embodiments, each of R⁴ and R⁵ is independently selected from hydrogen, optionally substituted C₁₋₆ alkyl, or optionally substituted C₃-6 carbocyclyl; or alternatively, R⁴ and R⁵ taken together form an optionally substituted 3-10 membered heterocyclyl. In several embodiments, X is Cl. In several embodiments, R² is-OR⁶ or optionally substituted (heterocyclyl)alkynyl. In several embodiments, R⁶ is selected from the group consisting of methyl, optionally substituted 2-10 membered heteroalkyl, and (heterocyclyl)alkyl. In several embodiments, R³ is selected from the group consisting of hydrogen, halogen, and —OMe. In several embodiments, R^{a} is hydrogen or optionally substituted C₁-C₁₀ alkyl. In several embodiments, the A ring is an optionally substituted heteroaryl.

FIG. 2 provides an exemplary synthesis of a compound of Formula (I). As shown in FIG. 2, in several embodiments, compounds of Formula (I) are prepared by one or more of the following steps: a first halogen (e.g., chloro) displacement, a second halogen (e.g., chloro) displacement, a coupling (e.g., palladium), and/or a third halogen (e.g., chloro) displacement. In several embodiments, the synthesis yields 2-amino and 4-amino functionalized regions of the 6,7-dialkoxyquinazoline scaffolds as depicted in the general Scheme 1 of FIG. 2. An exemplary set of conditions for Scheme 1 were as follows: (i) HNR^{a}(Ring A) (1.5 eq.), DIPEA (1-10 eq.), DMF. NaI, 70° C.; (ii) HNRₐ(Ring A) (1.05 eq.). K₂CO₃ (3 eq.), xanthphos (0.2 eq), Pd(OAc)₂ (0.15 eq.), DMF/THF. 70° C.; (iii) HR¹(5-20 eq.). DIPEA (6-10 eq.), DMF, iPrOH, 2-BuOH or combination, 90° ° C. convention heating.

Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmic, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

If the compounds of the present technology contain one or more chiral centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present disclosure were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

Trademarks used herein are examples only and reflect illustrative materials used at the time of the present disclosure. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the present disclosure.

### Examples

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### General Procedures

All reactions were carried out under an atmosphere of argon. Reagents and solvents were used from commercial sources without additional purification. Hydrogenation reactions were run under a balloon. Microwave reactions were performed using a CEM Discover SP microwave synthesizer. Sample purification was conducted on a Buchi Pureflash with ELSD purification system using pre-packed commercially available silica gel columns. Thin layer chromatography (TLC) was performed on aluminium plates using Merck Kiesegel 60 F254 (230-400 mesh) fluorescent treated silica which were visualized under ultraviolet light (254 nm), or by staining with potassium permanganate or ninhydrin solution as appropriate. All Nuclear Magnetic Resonance (NMR) spectra were acquired on a Bruker Avance III HD 400 MHZ NMR spectrometer; chemical shifts are reported in ppm (δ). HPLC/MS was performed on a Sciex 5500 Qtrap mass spectrometry coupled with Shidmazu Nexera X2 UHPLC using Phenomenex Luna C18 column (50×2.0 mm, 3 μm particle size) via following method: The gradient mobile phase A contains 0.1% formic acid in water and mobile phase B contains 0.1% formic acid in acetonitrile; A/B (95:5) from 0 to 0.9 minutes; to A/B (5:95) from 0.9 to 2.2 minutes; A/B (5:95) from 2.2 to 4.14 minutes; to A/B (95:5) from 4.14 to 4.20 minutes; A/B (95:5) from 4.2 to 6 minutes. The flow rate was 0.4 mL/min and the column temperature maintained at 35° C. and autosampler temperature at 4° C. Ion spray voltage, drying gas temperature, ion source gas 1, and ion source gas 2 settings were 4500V. 500° C., 35V, and 45V with ESI set in positive mode using full scan. All compounds purity was analyzed on Agilent 1260 Infinity II Lab LC Series HPLC (1260 Quat pum, 1260 vial autosampler, ICC column oven, 1260 DAD WR detector). Samples were injected into Phenomenex Synergi Polar RP column (150×4.6 mm, 4 μm, 80 Å). The gradient mobile phase (A: water with 0.1% trifluoroacetic acid, B: acetonitrile with 0.1% trifluoroacetic acid; A/B (99:1) from 0 minute; to A/B (1:99) from 0 to 15 minutes; A/B (1:99) from 15 to 18 minutes; A/B (99:1) from 18 to 18.1 minutes; A/B (99:1) from 18.1 to 20 minutes) pumped at a flow rate of 1 mL/min. UV detector was set to 254 nm with column oven at 35° C. Injection volume was 10 μL, unless otherwise specified. All compounds that were evaluated in biological assay had ≥90% and animal study had ≥95% purity.

### Example 1: Synthesis of 6-Methoxy-N-(5-methyl-1H-pyrazol-3-yl)-2-(pyrrolidin-1-yl)-7-(3-(pyrrolidin-1-yl)propoxy)quinazolin-4-amine, (Compound 1)

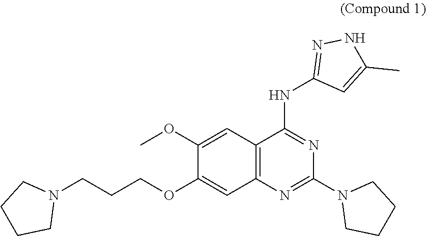 [SMILES: COc1cc2c(Nc3cc(C)[nH]n3)nc(N3CCCC3)nc2cc1OCCCN1CCCC1]

Preparation: To a solution of 2-chloro-6-methoxy-N-(5-methyl-1H-pyrazol-3-yl)-7-(3-(pyrrolidin-1-yl)propoxy)quinazolin-4-amine (0.09 g, 0.21 mmol), DIPEA (0.11 mL, 0.62 mmol), and pyrrolidine (0.07 g, 1.03 mmol) in anhydrous THF (3 mL)/2-butanol (1 mL) mixtures under argon. The tube was then sealed and heated to 90° C. for 3 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (24 g) with 95:5 CH₂Cl₂:MeOH w/2% 7N ammonia to give 6-methoxy-N-(5-methyl-1H-pyrazol-3-yl)-2-(pyrrolidin-1-yl)-7-(3-(pyrrolidin-1-yl)propoxy)quinazolin-4-amine (0.04 g, 47%) as a light brown solid. 1H NMR (400 MHZ, DMSO-d₆): δ 12.01 (bs, 1H), 9.83 (s, 1H), 7.78 (s, 1H), 6.75 (s, 1H), 6.70 (bs, 1H), 4.11 (t, 2H, J=8.0 Hz), 3.84 (s, 3H), 3.54 (m, 4H), 3.31 (m, 2H, partial masked under water), 2.55 (t, 2H, J=8.0 Hz), 2.46 (m, 4H), 2.25 (s, 3H), 1.93 (m, 7H), 1.70 (m, 4H). MS (ESI): Calcd. for C₂₄H₃₃F₂N₇O₂: 451, found 452 (M+H)⁺.

### Example 2: Synthesis of 2-Chloro-6-methoxy-N-(5-methyl-1H-pyrazol-3-yl)-7-(3-(pyrrolidin-1-yl)propoxy)quinazolin-4-amine, (compound 2)

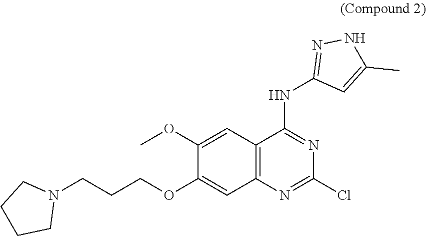 [SMILES: COc1cc2c(Nc3cc(C)[nH]n3)nc(Cl)nc2cc1OCCCN1CCCC1]

To a solution of commercially available 2,4-dichloro-6-methoxy-7-(3-(pyrrolidine-1-yl)propoxy)quinazoline (1.00 g, 2.81 mmol), K₂CO₃ (0.30, 3.09 mmol), and 3-amino-5-methyl-1H-pyrazole (0.76 g. 5.61 mmol) in anhydrous DMF (3 mL) under argon. The tube was then sealed and heated to 90° C. Upon completion after 2 days, the cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (80 g) with 95:5 CH₂Cl₂:MeOH w/2% 7N ammonia to give 2-chloro-6-methoxy-N-(5-methyl-1H-pyrazol-3-yl)-7-(3-(pyrrolidin-1-yl)propoxy)quinazolin-4-amine (0.19 g. 23%) as a white solid. ¹H NMR (400 MHz. DMSO-d₆): δ 12.24 (bs, 1H), 10.55 (s, 1H), 8.02 (s, 1H), 7.12 (s, 1H), 6.57 (bs, 1H), 4.17 (t, 2H, J=8.0 Hz), 3.92 (s, 3H), 2.57 (m, 2H), 2.49 (m, 4H, masked under DMSO-d), 2.27 (s, 3H), 1.96 (p, 2H, J=8.0 Hz), 1.70 (m, 4H). MS (ESI): Calcd. for C₂₀H₂₅ClN₆O₂: 416, found 417 (M+H)⁺.

### Example 3: Synthesis of 2-(4,4-difluoropiperidin-1-yl)-6-methoxy-N-(5-methyl-1H-pyrazol-3-yl)-7-(3-(pyrrolidin-1-yl)propoxy)quinazolin-4-amine (Compound 3)

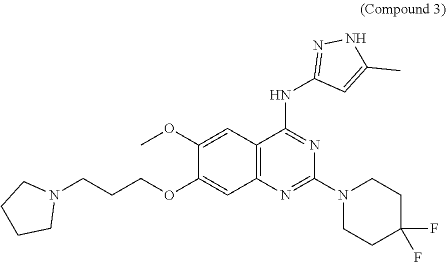 [SMILES: COc1cc2c(Nc3cc(C)[nH]n3)nc(N3CCC(F)(F)CC3)nc2cc1OCCCN1CCCC1]

To a solution of commercially available 2,4-dichloro-6-methoxy-7-(3-(pyrrolidine-1-yl)propoxy)quinazoline (0.20 g. 0.56 mmol), DIPEA (0.98 mL, 5.61 mmol), sodium iodide (0.09 g. 0.59 mmol), and 3-amino-5-methyl-1H-pyrazole (0.06 g. 0.59 mmol) in anhydrous DMF (3 mL) under argon. The tube was then sealed and heated to 50° C. The reaction was monitored by TLC and HPLC/MS of 417. Upon completion after 2 days, the cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The crude was dissolved 2-butanol (3 ml) followed by adding DIPEA (0.44 mL, 2.53 mmol) and 4,4-difluoropiperidine hydrochloride (0.33 g. 2.11 mmol). Then the sealed tube was heated to 90° C. for 4 days. The cooled mixtures were quenched with sat. NaHCO₃ and extracted with 8:2 dichloromethane/isopropanol mixtures (3×50 mL) then washed once with brine. The combined organic layers were dried over anhydrous Na₂SO₄, filtered, and concentrated in vacuo. The residue was purified by Buchi Pureflash chromatography over silica gel cartridge (24 g) with 95:5 CH₂Cl₂:MeOH w/2% 7N ammonia to give 2-(4,4-difluoropiperidin-1-yl)-6-methoxy-N-(5-methyl-1H-pyrazol-3-yl)-7-(3-(pyrrolidin-1-yl)propoxy)quinazolin-4-amine (0.06 g, 23%) as an off-white solid. ¹H NMR (400 MHZ, CDCl₃): δ 7.86 (s, 1H), 6.93 (s, 1H), 6.92 (s, 1H), 6.51 (bs, 1H), 4.18 (t, 2H, J=8.0 Hz), 4.01 (m, 4H), 3.86 (s, 3H), 2.34 (s. 3H), 2.15-1.95 (m, 7H), 1.77 (m, 4H). MS (ESI): Calcd. for C₂₅H₃₃F₂N₇O₂: 501, found 502 (M+H)⁺. ¹H NMR (400 MHZ, DMSO-d₆): δ 12.08 (bs, 1H), 9.89 (s, 1H), 7.81 (s, 1H), 6.80 (s, 1H), 6.43 (s, 1H), 4.10 (t, 2H, J=8.0 Hz), 3.90 (m, 4H), 3.84 (s, 3H), 2.53 (t, 2H, J=8.0 Hz, partial masked under DMSO-d₆), 2.44 (m, 4H), 2.26 (s, 3H), 2.02-1.91 (m, 6H), 1.69 (m, 4H). MS (ESI): Calcd. for C₂₅H₃₃F₂N₇O₂: 501, found 502 (M+H)⁺.

### Example 4: Synthesis of 2-(Azetidin-1-yl)-6-methoxy-N-(5-methyl-1H-pyrazol-3-yl)-7-(3-(pyrrolidin-1-yl)propoxy)quinazolin-4-amine, (Compound 4)

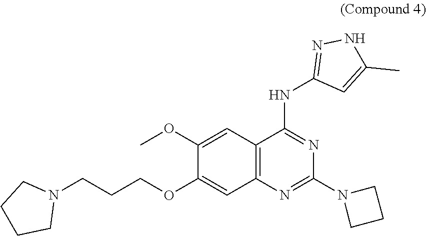 [SMILES: COc1cc2c(Nc3cc(C)[nH]n3)nc(N3CCC3)nc2cc1OCCCN1CCCC1]

Preparation: To a solution of 2-chloro-6-methoxy-N-(5-methyl-1H-pyrazol-3-yl)-7-(3-(pyrrolidin-1-yl)propoxy)quinazolin-4-amine (0.11 g, 0.26 mmol), DIPEA (0.13 mL, 0.77 mmol), and azetidine (0.07 g. 1.29 mmol) in anhydrous THF (3 mL)/2-butanol (1 mL) mixtures under argon. The tube was then sealed and heated to 90° C. for 3 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (24 g) with 95:5 CH₂Cl₂:MeOH w/2% 7N ammonia to give 2-(azetidin-1-yl)-6-methoxy-N-(5-methyl-1H-pyrazol-3-yl)-7-(3-(pyrrolidin-1-yl)propoxy)quinazolin-4-amine (0.04 g, 47%) as a white solid. ¹H NMR (400 MHz, DMSO-d₆): δ 12.01 (bs, 1H), 9.93 (s, 1H), 7.81 (s, 1H), 6.79 (s, 1H), 6.69 (bs, 1H), 4.08 (t, 2H, J=8.0 Hz), 4.02 (t, 4H, J=8.0 Hz), 3.84 (s, 3H), 3.31 (m, 2H, partial masked under water), 2.54 (t, 2H, J=8.0 Hz), 2.45 (m, 4H), 2.24 (s, 3H), 1.92 (p, 2H, J=4.0 Hz), 1.69 (m, 4H). MS (ESI): Calcd. for C₂₃H₃₁F₂N₇O₂: 437, found 438 (M+H)⁺.

### Example 5: Synthesis of N-(2-chloro-6-methoxy-7-(3-(pyrrolidin-1-yl)propoxy)quinazolin-4-yl)-5-methylthiazol-2-amine, (Compound 5)

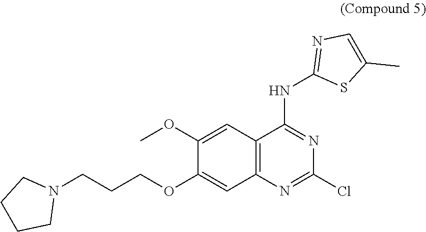 [SMILES: COc1cc2c(Nc3ncc(C)s3)nc(Cl)nc2cc1OCCCN1CCCC1]

Preparation: To a solution of commercially available 2,4-dichloro-6-methoxy-7-(3-(pyrrolidine-1-yl)propoxy)quinazoline (0.20 g, 0.56 mmol), K₂CO₃ (0.23 g, 1.68 mmol), 2-amino-5-methylthiazole (0.67 g, 0.59 mmol) and xantphos (0.03 g, 0.11 mmol) in anhydrous 1:1 DMF/THF (4 mL) bubble with argon for 40 min. Then palladium (II) acetate (0.01 g, 0.08 mmol) added and continue to bubble with argon for addition 15 min. The tube was then sealed and heated to 70° C. Upon completion in overnight (20 h), the cooled reaction was filtered through a pad of celite and concentrated in vacuo. The residue was purified by Buchi Pureflash chromatography over pre-neutralized silica gel cartridge (40 g) with 9:1 CH₂Cl₂:MeOH w/2% 7N ammonia to give N-(2-chloro-6-methoxy-7-(3-(pyrrolidin-1-yl)propoxy)quinazolin-4-yl)-5-methylthiazol-2-amine (0.10 g, 42%) as an orange solid. ¹H NMR (400 MHZ, DMSO-d₆): δ 12.05 (bs, 1H), 7.98 (s, 1H), 7.22 (d, 1H, J=0.5 Hz), 7.18 (s, 1H), 4.18 (t, 2H, J=8.0 Hz), 3.92 (s, 3H), 2.59 (t, 2H, J=8.0 Hz), 2.53 (m, 4H, partial masked under DMSO-d), 2.38 (s, 3H), 1.96 (p, 2H, J=8.0 Hz), 1.70 (m, 4H). MS (ESI): Calcd. for C₂₀H₂₄ClN₅O₂S: 433, found 434 (M+H)⁺.

### Example 6: Synthesis of N-(5-cyclopropyl-1H-pyrazol-3-yl)-2-(4,4-difluoropiperidin-1-yl)-6-methoxy-7-(3-(pyrrolidin-1-yl)propoxy)quinazolin-4-amine, (Compound 6)

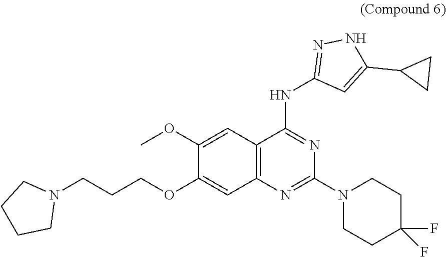 [SMILES: COc1cc2c(Nc3cc(C4CC4)[nH]n3)nc(N3CCC(F)(F)CC3)nc2cc1OCCCN1CCCC1]

Preparation: To a solution of commercially available 2,4-dichloro-6-methoxy-7-(3-(pyrrolidine-1-yl)propoxy)quinazoline (0.30 g, 0.84 mmol), DIPEA (0.44 mL, 2.53 mmol), sodium iodide (0.14 g, 0.93 mmol), and 3-cyclopropyl-1H-pyrazol-5-amine (0.15 g, 0.93 mmol) in anhydrous DMF (6 mL) under argon. The tube was then sealed and heated to 70° C. The reaction was monitored by TLC and HPLC/MS of 443. Upon completion after 2 days, then DIPEA (1.00 mL, 5.89 mmol) and 4,4-difluoropiperidine hydrochloride (0.66 g, 4.21 mmol) was added. The sealed tube was heated to 90° C. for 4 days. The cooled mixtures were quenched with sat. NaHCO₃ and extracted with 8:2 dichloromethane/isopropanol mixtures (3×50 mL) then washed once with brine. The combined organic layers were dried over anhydrous Na₂SO₄, filtered, and concentrated in vacuo. The residue was purified by Buchi Pureflash chromatography over pre-neutralized silica gel cartridge (40 g) with 8:2 CH₂Cl₂:MeOH w/2% 7N ammonia to give N-(5-cyclopropyl-1H-pyrazol-3-yl)-2-(4,4-difluoropiperidin-1-yl)-6-methoxy-7-(3-(pyrrolidin-1-yl)propoxy)quinazolin-4-amine (0.14 g, 31%) as a beige solid. ¹H NMR (400 MHZ, DMSO-d₆): δ 12.13 (s, 1H), 9.89 (s, 1H), 7.80 (s, 1H), 6.80 (s, 1H), 6.32 (s, 1H), 4.10 (t, 2H, J=8.0 Hz), 3.89 (m, 4H), 3.84 (s, 3H), 2.53 (t, 2H, J=8.0 Hz), 2.44 (m, 4H), 2.01=1.88 (m, 7H), 1.68 (m, 4H), 0.95 (m, 2H), 0.69 (m, 2H). MS (ESI): Calcd. for C₂₇H₃₅F₂N₇O₂: 527, found 528 (M+H)⁺.

### Example 7: Synthesis of N-(5-(tert-butyl)-1H-pyrazol-3-yl)-2-(4,4-difluoropiperidin-1-yl)-6-methoxy-7-(3-(pyrrolidin-1-yl)propoxy)quinazolin-4-amine, (Compound 7)

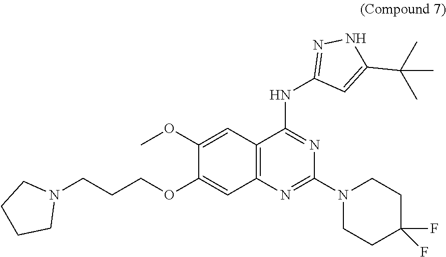 [SMILES: COc1cc2c(Nc3cc(C(C)(C)C)[nH]n3)nc(N3CCC(F)(F)CC3)nc2cc1OCCCN1CCCC1]

Preparation: To a solution of commercially available 2,4-dichloro-6-methoxy-7-(3-(pyrrolidine-1-yl)propoxy)quinazoline (0.30 g, 0.84 mmol), DIPEA (0.44 mL, 2.53 mmol), sodium iodide (0.14 g, 0.93 mmol), and 3-(tert-butyl)-1H-pyrazol-5-amine (0.13 g, 0.93 mmol) in anhydrous DMF (6 mL) under argon. The tube was then sealed and heated to 70° C. The reaction was monitored by TLC and HPLC/MS of 459. Upon completion after 2 days, then DIPEA (1.00 mL, 5.89 mmol) and 4,4-difluoropiperidine hydrochloride (0.66 g, 4.21 mmol) was added. The sealed tube was heated to 90° C. for 4 days. The cooled mixtures were quenched with sat. NaHCO₃ and extracted with 8:2 dichloromethane/isopropanol mixtures (3×50 mL) then washed once with brine. The combined organic layers were dried over anhydrous Na₂SO₄, filtered, and concentrated in vacuo. The residue was purified by Buchi Pureflash chromatography over pre-neutralized silica gel cartridge (40 g) with 8:2 CH₂Cl₂:MeOH w/2% 7N ammonia to give N-(5-(tert-butyl)-1H-pyrazol-3-yl)-2-(4,4-difluoropiperidin-1-yl)-6-methoxy-7-(3-(pyrrolidin-1-yl)propoxy)quinazolin-4-amine (0.15 g, 32%) as an off-white solid. ¹H NMR (400 MHZ, DMSO-d₆): δ 12.10 (s, 1H), 9.93 (s, 1H), 7.81 (s, 1H), 6.81 (s, 1H), 6.50 (s, 1H), 4.10 (t, 2H, J=8.0 Hz), 3.92 (m, 4H), 3.84 (s, 3H), 2.53 (t, 2H, J=8.0 Hz), 2.44 (m, 4H), 1.98 (m, 4H), 1.93 (p, 2H, J=8.0 Hz), 1.68 (m, 4H), 1.30 (s, 9H). MS (ESI): Calcd. for C₂₈H₃₉F₂N₇O₂: 543, found 544 (M+H)⁺.

### Example 8: Synthesis of 2-(4,4-difluoropiperidin-1-yl)-A-(5-ethyl-1H-pyrazol-3-yl)-6-methoxy-7-(3-(pyrrolidin-1-yl)propoxy)quinazolin-4-amine, (Compound 8)

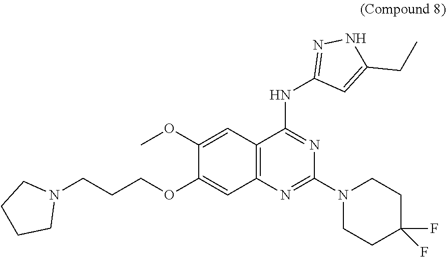 [SMILES: CCc1cc(Nc2nc(N3CCC(F)(F)CC3)nc3cc(OCCCN4CCCC4)c(OC)cc23)n[nH]1]

Preparation: To a solution of commercially available 2,4-dichloro-6-methoxy-7-(3-(pyrrolidine-1-yl)propoxy)quinazoline (0.30 g, 0.84 mmol), DIPEA (0.44 mL, 2.53 mmol), sodium iodide (0.14 g, 0.93 mmol), and 3-amino-5-ethyl-1H-pyrazole (0.1 g, 0.93 mmol) in anhydrous DMF (6 mL) under argon. The tube was then sealed and heated to 70° ° C. The reaction was monitored by TLC and HPLC/MS of 459. Upon completion after 2 days, then DIPEA (1.00 mL, 5.89 mmol) and 4,4-difluoropiperidine hydrochloride (0.66 g, 4.21 mmol) was added. The sealed tube was heated to 90° C. for 4 days. The cooled mixtures were quenched with sat. NaHCO₃ and extracted with 8:2 dichloromethane/isopropanol mixtures (3×50 mL) then washed once with brine. The combined organic layers were dried over anhydrous Na₂SO₄, filtered, and concentrated in vacuo. The residue was purified by Buchi Pureflash chromatography over pre-neutralized silica gel cartridge (40 g) with 8:2 CH₂Cl₂:MeOH w/2% 7N ammonia to give 2-(4,4-difluoropiperidin-1-yl)-N-(5-ethyl-1H-pyrazol-3-yl)-6-methoxy-7-(3-(pyrrolidin-1-yl)propoxy)quinazolin-4-amine (0.13 g, 31%) as a beige solid. ¹H NMR (400 MHZ, DMSO-d₆): δ 12.09 (s, 1H), 9.90 (s, 1H), 7.81 (s, 1H), 6.81 (s, 1H), 6.47 (s, 1H), 4.10 (t, 2H, J=8.0 Hz), 3.91 (m, 4H), 3.84 (s, 3H), 2.63 (q, 2H, J=8.0 Hz), 2.53 (t, 2H, J=8.0 Hz), 2.44 (m, 4H), 1.98 (m, 4H), 1.93 (p, 2H, J=8.0 Hz), 1.68 (m, 4H), 1.22 (t, 3H, J=8.0 Hz). MS (ESI): Calcd. for C₂₆H₃₅F₂N₇O₂: 515, found 516 (M+H)⁺.

### Example 9: Synthesis of N-(2-(4,4-difluoropiperidin-1-yl)-6-methoxy-7-(3-(pyrrolidin-1-yl)propoxy)quinazolin-4-yl)-5-methylthiazol-2-amine, (Compound 9)

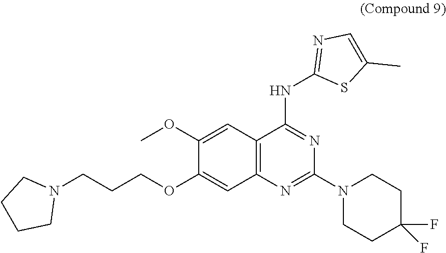 [SMILES: COc1cc2c(Nc3ncc(C)s3)nc(N3CCC(F)(F)CC3)nc2cc1OCCCN1CCCC1]

Preparation: To a solution of N-(2-chloro-6-methoxy-7-(3-(pyrrolidin-1-yl)propoxy)quinazolin-4-yl)-5-methylthiazol-2-amine (0.09 g, 0.21 mmol), DIPEA (0.36 mL, 2.07 mmol), and 4,4-difluoropiperidine hydrochloride (0.13 g, 0.83 mmol) in anhydrous DMF (3 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over pre-neutralized silica gel cartridge (24 g) with 8:2 CH₂Cl₂:MeOH w/2% 7N ammonia to give N-(2-(4,4-difluoropiperidin-1-yl)-6-methoxy-7-(3-(pyrrolidin-1-yl)propoxy)quinazolin-4-yl)-5-methylthiazol-2-amine (0.07 g, 62%) as a golden light brown solid. ¹H NMR (400 MHZ, DMSO-d₆): δ 11.62 (bs, 1H), 7.92 (s, 1H), 7.20 (d, 1H, J=0.5 Hz), 6.87 (s, 1H), 4.12 (t, 2H, J=8.0 Hz), 4.03 (m, 4H), 3.86 (s, 3H), 2.53 (t, 2H, J=8.0 Hz), 2.44 (m, 4H), 2.39 (d, 3H, J=4.0 Hz), 2.03 (m, 4H), 1.93 (p, 2H, J=8.0 Hz), 1.68 (m, 4H). MS (ESI): Calcd. for C₂₅H₃₂F₂N₆O₂S: 518, found 519 (M+H)⁺.

### Example 10: Synthesis of N-(5-cyclobutyl-1H-pyrazol-3-yl)-2-(4,4-difluoropiperidin-1-yl)-6-methoxy-7-(3-(pyrrolidin-1-yl)propoxy)quinazolin-4-amine, (Compound 10)

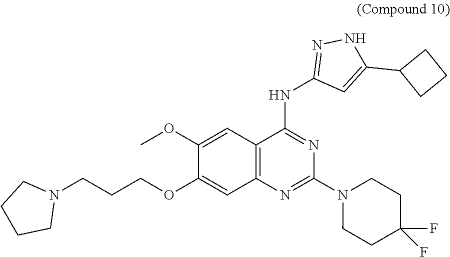 [SMILES: COc1cc2c(Nc3cc(C4CCC4)[nH]n3)nc(N3CCC(F)(F)CC3)nc2cc1OCCCN1CCCC1]

Preparation: To a solution of commercially available 2,4-dichloro-6-methoxy-7-(3-(pyrrolidine-1-yl)propoxy)quinazoline (0.30 g, 0.84 mmol), DIPEA (0.44 mL, 2.53 mmol), sodium iodide (0.14 g, 0.93 mmol), and 3-amino-5-cyclobutyl-1H-pyrazole (0.13 g, 0.93 mmol) in anhydrous DMF (6 mL) under argon. The tube was then sealed and heated to 70° C. The reaction was monitored by TLC and HPLC/MS of 457. Upon completion after 3 days, then DIPEA (1.00 mL, 5.89 mmol) and 4,4-difluoropiperidine hydrochloride (0.66 g. 4.21 mmol) was added. The sealed tube was heated to 90° C. for 4 days. The cooled mixtures were quenched with sat. NaHCO₃ and extracted with 8:2 dichloromethane/isopropanol mixtures (3×50 mL) then washed once with brine. The combined organic layers were dried over anhydrous Na₂SO₄, filtered, and concentrated in vacuo. The residue was purified by Buchi Pureflash chromatography over pre-neutralized silica gel cartridge (40 g) with 9:1 CH₂Cl₂:MeOH w/2% 7N ammonia to give N-(5-cyclobutyl-1H-pyrazol-3-yl)-2-(4,4-difluoropiperidin-1-yl)-6-methoxy-7-(3-(pyrrolidin-1-yl)propoxy)quinazolin-4-amine (0.13 g. 29%) as a beige solid. ¹H NMR (400 MHZ, DMSO-d₆): δ 12.13 (s, 1H), 9.92 (s, 1H), 7.81 (s, 1H), 6.81 (s, 1H), 6.54 (s, 1H), 4.10 (t, 2H, J=8.0 Hz), 3.92 (m, 4H), 3.84 (s, 3H), 3.51 (p. 1H, J=8.0 Hz), 2.53 (t, 2H, J=8.0 Hz), 2.44 (m, 4H), 2.32 (m, 2H), 2.11 (m, 2H), 2.03-1.86 (m, 8H), 1.68 (m, 4H). MS (ESI): Calcd. for C₂₈H₃₇F₂N₇O₂: 541, found 542 (M+H)⁺.

### Example 11: Synthesis of A-(2-chloro-6-methoxy-7-(3-(pyrrolidin-1-yl)propoxy)quinazolin-4-yl)-5-methyl-1,3,4-thiadiazol-2-amine, (Compound 11)

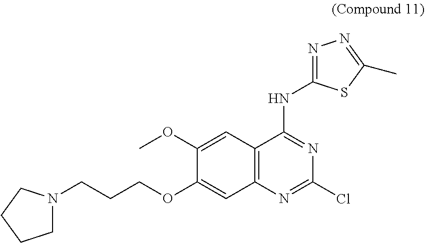 [SMILES: COc1cc2c(Nc3nnc(C)s3)nc(Cl)nc2cc1OCCCN1CCCC1]

Preparation: To a solution of commercially available 2,4-dichloro-6-methoxy-7-(3-(pyrrolidine-1-yl)propoxy)quinazoline (0.25 g, 0.70 mmol), K₂CO₃ (0.29 g, 2.11 mmol), 2-amino-5-1,3,4-thiadiazole (0.08 g. 0.74 mmol) and xantphos (0.08 g. 0.14 mmol) in anhydrous 1:1 DMF/THF (4 mL) bubble with argon for 40 min. Then palladium (II) acetate (0.02 g. 0.11 mmol) added and continue to bubble with argon for addition 15 min. The tube was then sealed and heated to 70° C. Upon completion in overnight (20 h), the cooled reaction was filtered through a pad of celite and concentrated in vacuo. The residue was purified by Buchi Pureflash chromatography over pre-neutralized silica gel cartridge (40 g) with 9:1 CH₂Cl₂:MeOH w/2% 7N ammonia to give N-(2-chloro-6-methoxy-7-(3-(pyrrolidin-1-yl)propoxy)quinazolin-4-yl)-5-methyl-1,3,4-thiadiazol-2-amine (0.16 g, 52%) as a yellow solid. ¹H NMR (400 MHZ, DMSO-d₆): δ NH (not observed), 7.86 (s, 1H), 7.10 (s, 1H), 4.18 (t, 2H, J=6.2 Hz), 3.91 (s, 3H), 2.87 (t, 2H, J=7.6 Hz), 2.82 (m, 4H), 2.59 (s, 3H), 2.05 (p, 2H, J=4.0 Hz), 1.80 (m, 4H). MS (ESI): Calcd. for C₁₉H₂₃ClN₆O₂S: 434, found 435 (M+H)⁺.

### Example 12: Synthesis of N-(2-(4,4-difluoropiperidin-1-yl)-6-methoxy-7-(3-(pyrrolidin-1-yl)propoxy)quinazolin-4-yl)-5-methyl-1,3,4-thiadiazol-2-amine, (Compound 12)

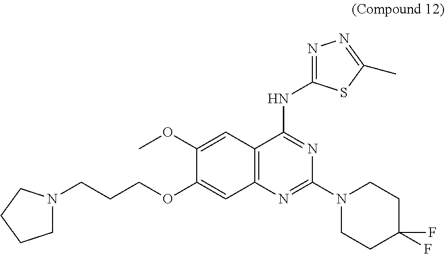 [SMILES: COc1cc2c(Nc3nnc(C)s3)nc(N3CCC(F)(F)CC3)nc2cc1OCCCN1CCCC1]

Preparation: To a solution of N-(2-chloro-6-methoxy-7-(3-(pyrrolidin-1-yl)propoxy)quinazolin-4-yl)-5-methyl-1,3,4-thiadiazol-2-amine (0.15 g, 0.34 mmol), DIPEA (0.60 mL, 3.45 mmol), and 4,4-difluoropiperidine hydrochloride (0.22 g, 1.38 mmol) in anhydrous DMF (6 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over pre-neutralized silica gel cartridge (24 g) with 8:2 CH₂Cl₂:MeOH w/2% 7N ammonia to give N-(2-(4,4-difluoropiperidin-1-yl)-6-methoxy-7-(3-(pyrrolidin-1-yl)propoxy)quinazolin-4-yl)-5-methyl-1,3,4-thiadiazol-2-amine (0.14 g, 80%) as a light yellow solid. ¹H NMR (400 MHZ, DMSO-d₆): δ NH (not observed), 7.91 (s, 1H), 6.88 (s, 1H), 4.13 (t, 2H, J=6.4 Hz), 4.00 (m, 4H), 3.87 (s, 3H), 2.55 (t, 2H, J=7.2 Hz), 2.47 (m, 4H), 2.02 (m, 4H), 1.94 (p, 2H, J=6.8 Hz), 1.69 (m, 4H). MS (ESI): Calcd. for C₂₄H₃₁F₂N₇O₂S: 519, found 520 (M+H)⁺.

### Example 13: Synthesis of 2-(4,4-difluoropiperidin-1-yl)-N-(5-isopropyl-1H-pyrazol-3-yl)-6-methoxy-7-(3-(pyrrolidin-1-yl)propoxy)quinazolin-4-amine, (Compound 13)

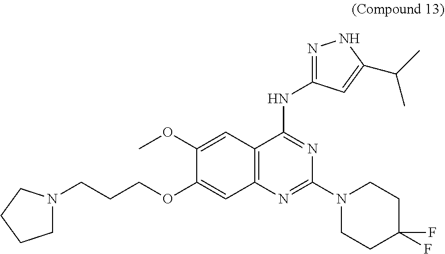 [SMILES: COc1cc2c(Nc3cc(C(C)C)[nH]n3)nc(N3CCC(F)(F)CC3)nc2cc1OCCCN1CCCC1]

Preparation: To a solution of commercially available 2,4-dichloro-6-methoxy-7-(3-(pyrrolidine-1-yl)propoxy)quinazoline (0.30 g, 0.84 mmol), DIPEA (0.44 mL, 2.53 mmol), sodium iodide (0.14 g, 0.93 mmol), and 5-isopropyl-1H-pyrazol-3-amine (0.12 g, 0.93 mmol) in anhydrous DMF (6 mL) under argon. The tube was then sealed and heated to 70° ° C. The reaction was monitored by TLC and HPLC/MS of 445. Upon completion after 3 days, then DIPEA (1.00 mL, 5.89 mmol) and 4,4-difluoropiperidine hydrochloride (0.66 g, 4.21 mmol) was added. The sealed tube was heated to 90° C. for 4 days. The cooled mixtures were quenched with sat. NaHCO₃ and extracted with 8:2 dichloromethane/isopropanol mixtures (3×50 mL) then washed once with brine. The combined organic layers were dried over anhydrous Na₂SO₄, filtered, and concentrated in vacuo. The residue was purified by Buchi Pureflash chromatography over pre-neutralized silica gel cartridge (40 g) with 9:1 CH₂Cl₂:MeOH w/2% 7N ammonia to give 2-(4,4-difluoropiperidin-1-yl)-N-(5-isopropyl-1H-pyrazol-3-yl)-6-methoxy-7-(3-(pyrrolidin-1-yl)propoxy)quinazolin-4-amine (0.12 g, 28%) as a beige solid. ¹H NMR (400 MHZ, DMSO-d₆): δ 12.09 (s, 1H), 9.92 (s, 1H), 7.81 (s, 1H), 6.81 (s, 1H), 6.48 (s, 1H), 4.10 (t, 2H, J=6.4 Hz), 3.91 (m, 4H), 3.84 (s, 3H), 2.95 (sept, 2H, J=6.8 Hz), 2.53 (t, 2H, J=7.2 Hz), 2.44 (m, 4H), 1.98 (m, 4H), 1.92 (m, 2H), 1.67 (m, 4H), 1.25 (d, 6H, J=6.8 Hz). MS (ESI): Calcd. for C₂₇H₃₇F₂N₇O₂: 529, found 530 (M+H)⁺.

### Example 14: Synthesis of 2-(4,4-difluoropiperidin-1-yl)-6-methoxy-A-methyl-N-(5-methyl-1H-pyrazol-3-yl)-7-(3-(pyrrolidin-1-yl)propoxy)quinazolin-4-amine, (Compound 14)

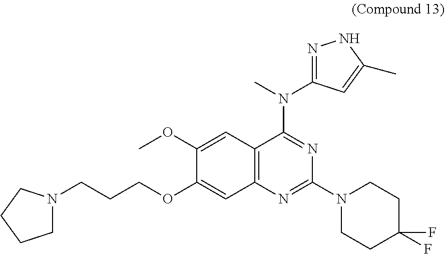 [SMILES: COc1cc2c(N(C)c3cc(C)[nH]n3)nc(N3CCC(F)(F)CC3)nc2cc1OCCCN1CCCC1]

Preparation: To a solution of commercially available 2,4-dichloro-6-methoxy-7-(3-(pyrrolidine-1-yl)propoxy)quinazoline (0.30 g, 0.84 mmol), DIPEA (0.44 mL, 2.53 mmol), sodium iodide (0.14 g, 0.93 mmol), and N,5-dimethyl-1H-pyrazol-3-amine (0.10 g, 0.93 mmol) in anhydrous DMF (6 mL) under argon. The tube was then sealed and heated to 70° C. The reaction was monitored by TLC and HPLC/MS of 431. Upon completion after 3 days, then DIPEA (1.00 mL, 5.89 mmol) and 4,4-difluoropiperidine hydrochloride (0.66 g, 4.21 mmol) was added. The sealed tube was heated to 90° C. for 4 days. The cooled mixtures were quenched with sat. NaHCO₃ and extracted with 8:2 dichloromethane/isopropanol mixtures (3×50 mL) then washed once with brine. The combined organic layers were dried over anhydrous Na₂SO₄, filtered, and concentrated in vacuo. The residue was purified by Buchi Pureflash chromatography over pre-neutralized silica gel cartridge (80 g) with 9:1 CH₂Cl₂:MeOH w/2% 7N ammonia to give 2-(4,4-difluoropiperidin-1-yl)-6-methoxy-N-methyl-N-(5-methyl-1H-pyrazol-3-yl)-7-(3-(pyrrolidin-1-yl)propoxy)quinazolin-4-amine (0.15 g, 35%) as an off-white solid. ¹H NMR (400 MHZ, DMSO-d₆): δ 12.39 (s, 1H), 6.78 (s, 1H), 6.28 (s, 1H), 5.90 (s, 1H), 4.06 (t, 2H, J=6.4 Hz), 3.38 (s, 3H), 3.35 (s, 3H), 2.53 (m, 2H), 2.46 m, 4H), 2.23 (s, 3H), 2.01 (m, 4H), 1.90 (p, 2H, J=6.4 Hz), 1.69 (m, 4H). MS (ESI): Calcd. for C₂₆H₃₅F₂N₇O₂: 515, found 516 (M)⁺.

### Example 15: Synthesis of 2-chloro-N-(5-cyclopropyl-1H-pyrazol-3-yl)-6-methoxy-7-(3-(pyrrolidin-1-yl)propoxy)quinazolin-4-amine, (Compound 15)

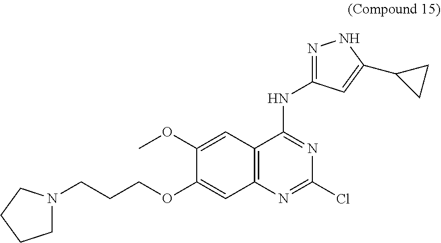 [SMILES: COc1cc2c(Nc3cc(C4CC4)[nH]n3)nc(Cl)nc2cc1OCCCN1CCCC1]

Preparation: To a solution of commercially available 2,4-dichloro-6-methoxy-7-(3-(pyrrolidine-1-yl)propoxy)quinazoline (4.00 g, 11.23 mmol), DIPEA (4.89 mL, 18.07 mmol), sodium iodide (3.63 g, 13.47 mmol), and 3-cyclopropyl-1H-pyrazol-5-amine (1.66 g, 13.47 mmol) in anhydrous DMF (40 mL) under argon. The tube was then sealed and heated to 70° C. Upon completion after 24 hours, the cooled reaction was slowly poor into cold water (400 mL) and the crude precipitate was collected by filtration. The crude residue was purified by Buchi Pureflash chromatography over silica gel cartridge (330 g) with 95:5 CH₂Cl₂:MeOH w/2% 7N ammonia to give 2-chloro-N-(5-cyclopropyl-1H-pyrazol-3-yl)-6-methoxy-7-(3-(pyrrolidin-1-yl)propoxy)quinazolin-4-amine (2.13 g, 43%) as a light brown solid. ¹H NMR (400 MHZ, DMSO-d₆): δ 12.26 (s, 1H), 10.53 (s, 1H), 8.00 (s, 1H), 7.11 (s, 1H), 6.50 (s, 1H), 4.16 (t, 2H, J=6.4 Hz), 3.92 (s, 3H), 2.54 (t, 2H, J=7.2 Hz), 2.42 (m, 4H), 1.94 (m, 3H), 1.68 (m, 4H), 0.95 (m, 2H), 0.72 (m, 2H). MS (ESI): Calcd. for C₂₂H₂₇ClN₆O₂: 442, found 443 (M+H)⁺.

### Example 16: Synthesis of 2-chloro-N-(5-ethyl-1H-pyrazol-3-yl)-6-methoxy-7-(3-(pyrrolidin-1-yl)propoxy)quinazolin-4-amine, (Compound 16)

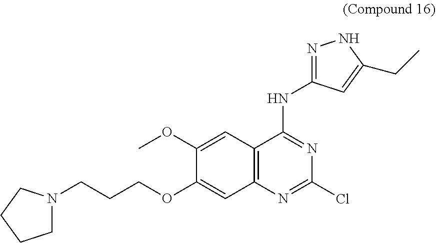 [SMILES: CCc1cc(Nc2nc(Cl)nc3cc(OCCCN4CCCC4)c(OC)cc23)n[nH]1]

Preparation: To a solution of commercially available 2,4-dichloro-6-methoxy-7-(3-(pyrrolidine-1-yl)propoxy)quinazoline (4.00 g, 11.23 mmol), DIPEA (4.89 mL. 18.07 mmol), sodium iodide (3.63 g, 13.47 mmol), and 3-amino-5-ethyl-1H-pyrazole (1.50 g. 13.47 mmol) in anhydrous DMF (40 mL) under argon. The tube was then sealed and heated to 70° C. Upon completion after 24 hours, the cooled reaction was slowly poor into cold water (400 mL) and the crude precipitate was collected by filtration. The crude residue was purified by Buchi Pureflash chromatography over silica gel cartridge (330 g) with 95:5 CH₂Cl₂:MeOH w/2% 7N ammonia to give 2-chloro-N-(5-ethyl-1H-pyrazol-3-yl)-6-methoxy-7-(3-(pyrrolidin-1-yl)propoxy)quinazolin-4-amine (1.91 g, 40%) as a light brown solid. ¹H NMR (400 MHZ, DMSO-d₆): δ 12.27 (s, 1H), 10.55 (s, 1H), 8.02 (s, 1H), 7.11 (s. 1H), 6.60 (s, 1H), 4.158 (t, 2H, J=6.4 Hz), 3.92 (s, 3H), 2.64 (quartet, 2H, J=7.6 Hz), 2.54 (t, 2H, J=7.2 Hz), 2.44 (m, 4H), 1.94 (quintet, 2H, J=6.8 Hz), 1.68 (m, 4H), 1.23 (t, 3H, J=7.6 Hz). MS (ESI): Calcd. for C₂₁H₂₇ClN₆O₂: 430, found 431 (M+H)⁺.

### Example 17: Synthesis of 2-chloro-6-methoxy-N-(5-(methoxymethyl)-1H-pyrazol-3-yl)-7-(3-(pyrrolidin-1-yl)propoxy)quinazolin-4-amine, (Compound 17)

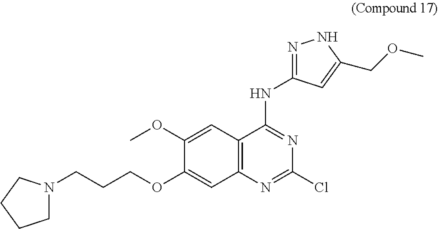 [SMILES: COCc1cc(Nc2nc(Cl)nc3cc(OCCCN4CCCC4)c(OC)cc23)n[nH]1]

Preparation: To a solution of commercially available 2,4-dichloro-6-methoxy-7-(3-(pyrrolidine-1-yl)propoxy)quinazoline (3.00 g. 8.42 mmol), DIPEA (3.67 mL, 21.05 mmol), sodium iodide (1.51 g, 10.11 mmol), and 5-(methoxymethyl)-1H-pyrazole-3-amine (1.28 g. 10.11 mmol) in anhydrous DMF (30 mL) under argon. The tube was then sealed and heated to 70° C. Upon completion after 24 hours, the cooled reaction was slowly poor into cold water (350 mL) and the crude precipitate was collected by filtration. The crude residue was purified by Buchi Pureflash chromatography over silica gel cartridge (330 g) with 95:5 CH₂Cl₂:MeOH w/2% 7N ammonia to give 2-chloro-6-methoxy-N-(5-(methoxymethyl)-1H-pyrazol-3-yl)-7-(3-(pyrrolidin-1-yl)propoxy)quinazolin-4-amine (1.29 g, 34%) as a beige solid. ¹H NMR (400 MHZ, DMSO-d₆): δ 12.63 (bs, 1H), 10.63 (bs, 1H), 7.98 (s, 1H), 7.09 (s, 1H), 6.77 (s, 1H), 4.45 (s, 2H), 4.13 (t, 2H, J=6.8 Hz), 3.85 (s, 3H), 3.29 (s, 3H), 2.62 (m, 6H), 1.74 (p, 2H, J=6.8 Hz), 1.68 (m, 4H). MS (ESI): Calcd. for C₂₁H₂₇ClN₆O₃: 446, found 446 (M)⁺.

### Example 18: Synthesis of M-(5-ethyl-1H-pyrazol-3-yl)-6-methoxy-A2 A2-dimethyl-7-(3-(pyrrolidin-1-yl)propoxy)quinazoline-2,4-diamine, (Compound 18)

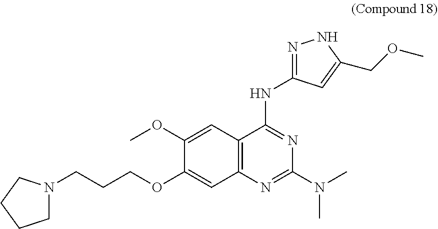 [SMILES: COCc1cc(Nc2nc(N(C)C)nc3cc(OCCCN4CCCC4)c(OC)cc23)n[nH]1]

Preparation: To a solution of 2-chloro-N-(5-ethyl-1H-pyrazol-3-yl)-6-methoxy-7-(3-(pyrrolidin-1-yl)propoxy)quinazolin-4-amine (0.20 g, 0.46 mmol), DIPEA (0.81 mL, 4.64 mmol), and dimethylamine hydrochloride (0.15 g, 1.86 mmol) in anhydrous DMF (5 mL) under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂C₁₂:MeOH w/2% 7N ammonia to give N⁴-(5-ethyl-1H-pyrazol-3-yl)-6-methoxy-N²,N²-dimethyl-7-(3-(pyrrolidin-1-yl)propoxy)quinazoline-2,4-diamine (0.12 g, 58%) as a light yellow solid. 1H NMR (400 MHZ, DMSO-d₆): δ 12.05 (bs, 1H), 9.83 (s, 1H), 7.78 (s, 1H), 6.76 (s, 1H), 6.62 (bs, 1H), 4.09 (t, 2H, J=6.8 Hz), 3.84 (s, 3H), 3.14 (s, 6H), 2.61 (q, 2H, J=7.2 Hz), 2.54 (t, 2H, J=6.8 Hz), 2.47 (m, 4H), 1.93 (p, 1H, J=6.8 Hz), 1.69 (m, 4H), 1.22 (t, 3H, J=7.6 Hz). MS (ESI): Calcd. for C₂₃H₃₃N₇O₂: 439.6, found 439.6 (M)⁺.

### Example 19: Synthesis of N-(5-ethyl-1H-pyrazol-3-yl)-6-methoxy-2-(pyrrolidin-1-yl)-7-(3-(pyrrolidin-1-yl)propoxy)quinazolin-4-amine, (Compound 19)

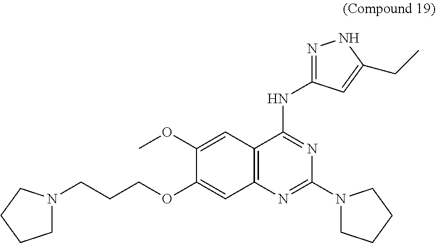 [SMILES: CCc1cc(Nc2nc(N3CCCC3)nc3cc(OCCCN4CCCC4)c(OC)cc23)n[nH]1]

Preparation: To a solution of 2-chloro-N-(5-ethyl-1H-pyrazol-3-yl)-6-methoxy-7-(3-(pyrrolidin-1-yl)propoxy)quinazolin-4-amine (0.20 g. 0.46 mmol), DIPEA (0.81 mL. 4.64 mmol), and pyrrolidine (0.17 g, 1.91 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give N-(5-ethyl-1H-pyrazol-3-yl)-6-methoxy-2-(pyrrolidin-1-yl)-7-(3-(pyrrolidin-1-yl)propoxy)quinazolin-4-amine (0.12 g, 54%) as a biege solid. ¹H NMR (400 MHz, DMSO-d₆): δ 12.02 (bs, 1H), 9.84 (s, 1H), 7.79 (s, 1H), 6.76 (bs, 1H), 6.75 (s, 1H), 4.09 (t, 2H, J=6.8 Hz), 3.54 (m, 4H), 2.61 (q. 2H, J=7.6 Hz), 2.55 (t. 2H, J=6.8 Hz), 2.46 (m, 4H), 1.92 (m. 6H), 1.69 (m, 4H), 1.22 (t, 3H, J=7.6 Hz). MS (ESI): Calcd. for C₂₅H₃₅N₇O₂: 465.6, found 465.8 (M)⁺.

### Example 20: Synthesis of N-(5-ethyl-1H-pyrazol-3-yl)-6-methoxy-2-morpholino-7-(3-(pyrrolidin-1-yl)propoxy)quinazolin-4-amine, (Compound 20)

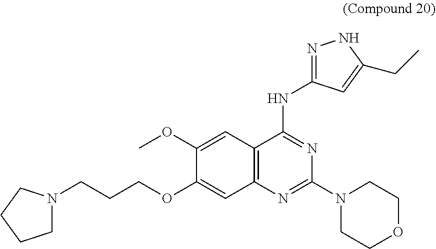 [SMILES: CCc1cc(Nc2nc(N3CCOCC3)nc3cc(OCCCN4CCCC4)c(OC)cc23)n[nH]1]

Preparation: To a solution of 2-chloro-N-(5-ethyl-1H-pyrazol-3-yl)-6-methoxy-7-(3-(pyrrolidin-1-yl)propoxy)quinazolin-4-amine (0.20 g. 0.46 mmol), DIPEA (0.81 mL, 4.64 mmol), and morpholine (0.20 g. 2.32 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give N-(5-ethyl-1H-pyrazol-3-yl)-6-methoxy-2-morpholino-7-(3-(pyrrolidin-1-yl)propoxy)quinazolin-4-amine (0.06 g, 25%) as a biege solid. ¹H NMR (400 MHz, DMSO-d₆): δ 12.08 (bs, 1H), 9.88 (s, 1H), 7.80 (s, 1H), 6.49 (bs, 1H), 4.09 (t, 2H, J=6.8 Hz), 3.84 (s, 3H), 3.68 (m, 7H), 2.61 (q, 2H, J=7.8 Hz), 2.44 (m, 4H), 1.92 (p, 2H, J=6.8 Hz), 1.68 (m, 4H), 1.21 (t, 3H, J=7.6 Hz). MS (ESI): Calcd. for C₂₅H₃₅N₇O₃: 481.6, found 481.6 (M)⁺.

### Example 21: Synthesis of A-(5-ethyl-1H-pyrazol-3-yl)-6-methoxy-2-(4-methoxypiperidin-1-yl)-7-(3-(pyrrolidin-1-yl)propoxy)quinazolin-4-amine, (Compound 21)

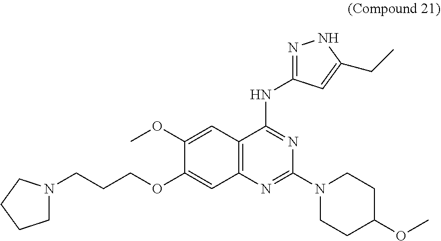 [SMILES: CCc1cc(Nc2nc(N3CCC(OC)CC3)nc3cc(OCCCN4CCCC4)c(OC)cc23)n[nH]1]

Preparation: To a solution of 2-chloro-N-(5-ethyl-1H-pyrazol-3-yl)-6-methoxy-7-(3-(pyrrolidin-1-yl)propoxy)quinazolin-4-amine (0.20 g, 0.46 mmol), DIPEA (0.81 mL, 4.64 mmol), and 4-methoxypiperidine (0.21 g, 1.86 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give N-(5-ethyl-1H-pyrazol-3-yl)-6-methoxy-2-(4-methoxypiperidin-1-yl)-7-(3-(pyrrolidin-1-yl)propoxy)quinazolin-4-amine (0.09 g, 39%) as a biege solid. ¹H NMR (400 MHZ, DMSO-d₆): δ 12.06 (bs, 1H), 9.83 (s, 1H), 7.78 (s, 1H), 6.76 (s, 1H), 6.49 (bs, 1H), 4.28 (m, 2H), 4.09 (t, 2H, J=6.8 Hz), 3.82 (s, 3H), 3.44-3.25 (m, 6H, partially masked under water), 3.24 (s, 3H), 2.62 (q, 2H, J=7.6 Hz), 2.56 (t, 2H, J=6.8H), 2.48 (m, 4H), 1.93 (p, 2H, J=6.8 Hz), 1.88 (m, 2H), 1.70 (m, 4H), 1.38 (m, 2H), 1.22 (t, 3H, J=7.6 Hz). MS (ESI): Calcd. for C₂₇H₃₉N₇O₃: 509.6, found 509.6 (M)⁺.

### Example 22: Synthesis of N₄-(5-cyclopropyl-1H-pyrazol-3-yl)-6-methoxy-N²,N²-dimethyl-7-(3-(pyrrolidin-1-yl)propoxy)quinazoline-2,4-diamine, (Compound 22)

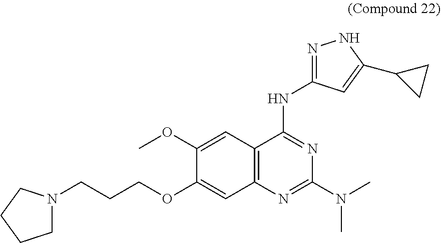 [SMILES: COc1cc2c(Nc3cc(C4CC4)[nH]n3)nc(N(C)C)nc2cc1OCCCN1CCCC1]

Preparation: To a solution of 2-chloro-N-(5-cyclopropyl-1H-pyrazol-3-yl)-6-methoxy-7-(3-(pyrrolidine-1-yl)propoxy)quinazolin-4-amine (0.20 g, 0.45 mmol), DIPEA (0.77 mL, 4.52 mmol), and dimethylamine hydrochloride (0.15 g, 1.81 mmol) in anhydrous DMF (5 mL) under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give N⁴-(5-cyclopropyl-1H-pyrazol-3-yl)-6-methoxy-N²,N²-dimethyl-7-(3-(yrrolidine-1-yl)propoxy)quinazoline-2,4-diamine (0.12 g, 58%) as a light yellow solid. ¹H NMR (400 MHZ, DMSO-d₆): δ 12.09 (bs, 1H), 9.81 (s, 1H), 7.77 (s, 1H), 6.76 (s, 1H), 6.50 (bs, 1H), 4.09 (t, 2H, J=6.8 Hz), 3.83 (s, 3H), 3.13 (s, 6H), 2.55 (t, 2H, J=6.8 Hz), 2.46 (m, 4H), 1.90 (m, 3H), 0.95 (m, 2H), 0.68 (m, 2H). MS (ESI): Calcd. for C₂₄H₃₃N₇O₂: 451.6, found 451.8 (M)⁺.

### Example 23: Synthesis of N-(5-cyclopropyl-1H-pyrazol-3-yl)-6-methoxy-2-(pyrrolidin-1-yl)-7-(3-(pyrrolidin-1-yl)propoxy)quinazolin-4-amine, (Compound 23)

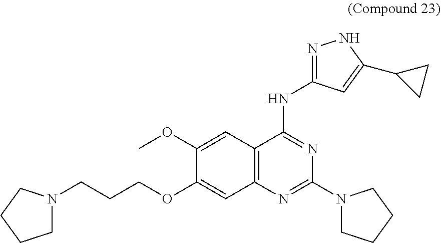 [SMILES: COc1cc2c(Nc3cc(C4CC4)[nH]n3)nc(N3CCCC3)nc2cc1OCCCN1CCCC1]

Preparation: To a solution of 2-chloro-N-(5-cyclopropyl-1H-pyrazol-3-yl)-6-methoxy-7-(3-(pyrrolidin-1-yl)propoxy)quinazolin-4-amine (0.20 g, 0.45 mmol), DIPEA (0.77 mL, 4.52 mmol), and pyrrolidine (0.16 g, 2.26 mmol) in THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give N-(5-cyclopropyl-1H-pyrazol-3-yl)-6-methoxy-2-(pyrrolidin-1-yl)-7-(3-(pyrrolidin-1-yl)propoxy)quinazolin-4-amine (0.07 g, 34%) as a beige solid. 1H NMR (400 MHZ, DMSO-d₆): δ 12.05 (bs, 1H), 9.84 (s, 1H), 7.77 (s, 1H), 6.75 (s, 1H), 6.63 (bs, 1H), 4.09 (t, 2H, J=6.8 Hz), 3.83 (s, 3H), 3.53 (m, 4H), 2.56 (t, 2H, J=6.8 Hz), 2.47 (m, 4H), 1.96-1.85 (m, 7H), 1.69 (m, 4H), 0.94 (m, 2H), 0.68 (m, 2H). MS (ESI): Calcd. for C₂₆H₃₅N₇O₂: 477.6, found 477.8 (M)⁺.

### Example 24: Synthesis of N-(5-cyclopropyl-1H-pyrazol-3-yl)-6-methoxy-2-morpholino-7-(3-(pyrrolidin-1-yl)propoxy)quinazolin-4-amine, (Compound 24)

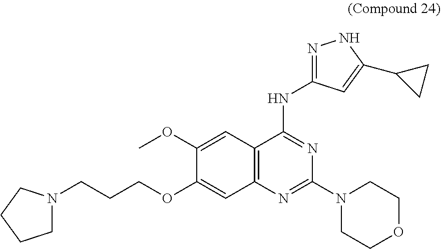 [SMILES: COc1cc2c(Nc3cc(C4CC4)[nH]n3)nc(N3CCOCC3)nc2cc1OCCCN1CCCC1]

Preparation: To a solution of 2-chloro-N-(5-cyclopropyl-1H-pyrazol-3-yl)-6-methoxy-7-(3-(pyrrolidin-1-yl)propoxy)quinazolin-4-amine (0.20 g, 0.45 mmol), DIPEA (0.77 mL, 4.52 mmol), and morpholine (0.16 g, 2.26 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give N-(5-cyclopropyl-1H-pyrazol-3-yl)-6-methoxy-2-morpholino-7-(3-(pyrrolidin-1-yl)propoxy)quinazolin-4-amine (0.08 g, 37%) as a beige solid. ¹H NMR (400 MHZ, DMSO-d₆): δ 12.11 (bs, 1H), 9.86 (s, 1H), 7.79 (s, 1H), 6.78 (s. 1H), 6.36 (bs, 1H), 4.09 (t. 2H, J=6.8 Hz), 3.84 (s, 3H), 3.67 (bs, 8H), 2.53 (t, 2H, J=6.8 Hz), 2.44 (m, 4H), 1.91 (m, 3H), 1.68 (m, 4H), 0.92 (m, 2H), 0.68 (m, 2H). MS (ESI): Calcd. for C₂₆H₃₅N₇O₂: 493.6 found 493.8 (M)⁺.

### Example 25: Synthesis of N-(5-cyclopropyl-1H-pyrazol-3-yl)-6-methoxy-2-(4-methoxypiperidin-1-yl)-7-(3-(pyrrolidin-1-yl)propoxy)quinazolin-4-amine, (Compound 25)

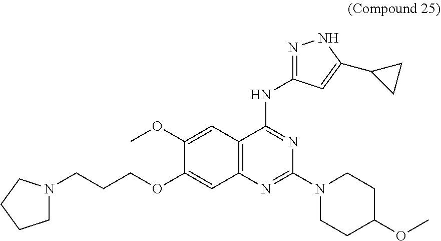 [SMILES: COc1cc2c(Nc3cc(C4CC4)[nH]n3)nc(N3CCC(OC)CC3)nc2cc1OCCCN1CCCC1]

Preparation: To a solution of 2-chloro-N-(5-cyclopropyl-1H-pyrazol-3-yl)-6-methoxy-7-(3-(pyrrolidin-1-yl)propoxy)quinazolin-4-amine (0.20 g. 0.45 mmol), DIPEA (0.77 mL, 4.52 mmol), and 4-methoxypiperidine (0.21 g. 1.81 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give N-(5-cyclopropyl-1H-pyrazol-3-yl)-6-methoxy-2-(4-methoxypiperidin-1-yl)-7-(3-(pyrrolidin-1-yl)propoxy)quinazolin-4-amine (0.08 g. 34%) as a beige solid. ¹H NMR (400 MHZ, DMSO-d₆): δ 12.12 (bs, 1H), 9.81 (s. 1H), 7.77 (s, 1H), 6.76 (s, 1H), 6.35 (bs, 1H), 4.27 (m, 2H), 4.09 (t, 2H, J=6.8 Hz), 3.83 (s, 3H), 3.41 (m, 1H), 3.31-3.22 (m, 4H), 3.28 (s, 3H), 2.53 (t, 2H, J=6.8 Hz), 2.44 (m, 4H), 1.90 (m, 5H), 1.68 (m, 4H), 1.36 (m, 2H), 0.95 (m, 2H), 0.66 (m, 2H). MS (ESI): Calcd. for C₂₈H₃₉N₇O₃: 521.7 found 521.8 (M)⁺.

### Example 26: Synthesis of 6-methoxy-M-(5-(methoxymethyl)-1H-pyrazol-3-yl)-N₂,N₂-dimethyl-7-(3-(pyrrolidin-1-yl)propoxy)quinazoline-2,4-diamine, (Compound 26)

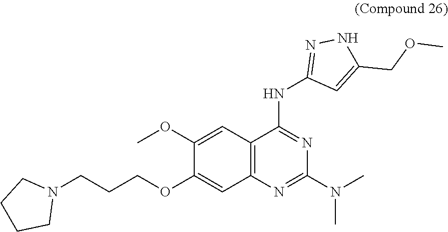 [SMILES: COCc1cc(Nc2nc(N(C)C)nc3cc(OCCCN4CCCC4)c(OC)cc23)n[nH]1]

Preparation: To a solution of 2-chloro-6-methoxy-N-(5-(methoxymethyl)-1H-pyrazol-3-yl)-7-(3-(pyrrolidin-1-yl)propoxy)quinazolin-4-amine (0.20 g, 0.45 mmol), DIPEA (0.78 mL, 4.47 mmol), and dimethylamine hydrochloride (0.15 g, 1.79 mmol) in anhydrous DMF (5 mL) under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give 6-methoxy-N⁴-(5-(methoxymethyl)-1H-pyrazol-3-yl)-N²,N²-dimethyl-7-(3-(pyrrolidin-1-yl)propoxy)quinazoline-2,4-diamine (0.01 g, 7%) as a light yellow solid. ¹H NMR (400 MHZ, DMSO-d₆): δ12.43 (bs, 1H), 9.90 (bs, 1H), 7.78 (s, 1H), 7.02 (s, 1H), 6.78 (s, 1H), 4.42 (s, 2H), 4.13 (t, 2H, J=6.8 Hz), 3.80 (s, 3H), 3.13 (s, 3H), 2.54 (s, 6H), 2.44 (m, 6H), 1.93 (p, 2H, J=6.8 Hz), 1.69 (m, 4H). MS (ESI): Calcd. for C₂₃H₃₃N₇O₃: 455.6, found 455.9 (M)⁺.

### Example 27: Synthesis of 6-methoxy-N-(5-(methoxymethyl)-1H-pyrazol-3-yl)-2-(pyrrolidin-1-yl)-7-(3-(pyrrolidin-1-yl)propoxy)quinazolin-4-amine, (Compound 27)

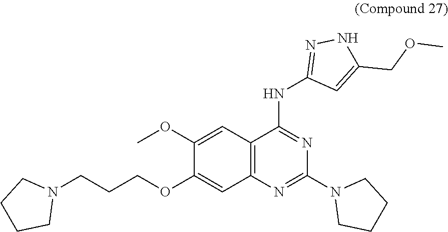 [SMILES: COCc1cc(Nc2nc(N3CCCC3)nc3cc(OCCCN4CCCC4)c(OC)cc23)n[nH]1]

Preparation: To a solution of 2-chloro-6-methoxy-N-(5-(methoxymethyl)-1H-pyrazol-3-yl)-7-(3-(pyrrolidin-1-yl)propoxy)quinazolin-4-amine (0.20 g. 0.45 mmol), DIPEA (0.78 mL, 4.47 mmol), and pyrrolidine (0.16 g. 2.24 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give 6-methoxy-N-(5-(methoxymethyl)-1H-pyrazol-3-yl)-2-(pyrrolidin-1-yl)-7-(3-(pyrrolidin-1-yl)propoxy)quinazolin-4-amine (0.02 g. 10%) as a beige solid. ¹H NMR (400 MHZ, DMSO-d₆): δ 12.40 (bs, 1H), 9.91 (bs, 1H), 7.78 (s, 1H), 7.01 (s, 1H), 6.77 (s, 1H), 4.41 (s, 2H), 4.09 (t, 2H, J=6.8 Hz), 3.80 (s, 3H), 3.54 (m, 4H), 3.20 (s, 3H), 2.43 (m, 6H), 1.92 (m, 4H), 1.86 (p. 2H, J=6.8 Hz), 1.68 (m, 4H). MS (ESI): Calcd. for C₂₅H₃₅N₇O₃: 481.6, found 481.8 (M)⁺.

### Example 28: Synthesis of 6-methoxy-N-(5-(methoxymethyl)-1H-pyrazol-3-yl)-2-morpholino-7-(3-(pyrrolidin-1-yl)propoxy)quinazolin-4-amine, (Compound 28)

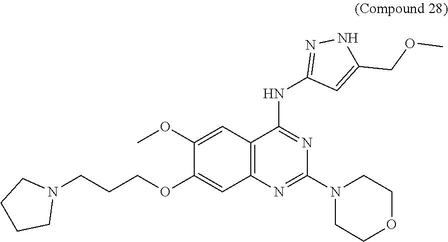 [SMILES: COCc1cc(Nc2nc(N3CCOCC3)nc3cc(OCCCN4CCCC4)c(OC)cc23)n[nH]1]

Preparation: To a solution of 2-chloro-6-methoxy-N-(5-(methoxymethyl)-1H-pyrazol-3-yl)-7-(3-(pyrrolidin-1-yl)propoxy)quinazolin-4-amine (0.20 g. 0.45 mmol), DIPEA (0.78 mL, 4.47 mmol), and morpholine (0.19 g. 2.24 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give 6-methoxy-N-(5-(methoxymethyl)-1H-pyrazol-3-yl)-2-morpholino-7-(3-(pyrrolidin-1-yl)propoxy)quinazolin-4-amine (0.03 g, 12%) as a light yellow solid. ¹H NMR (400 MHZ, DMSO-d₆): δ 12.45 (bs, 1H), 9.94 (bs, 1H), 7.79 (s, 1H), 7.04 (s, 1H), 6.78 (s, 1H), 4.42 (s, 2H), 4.09 (t, 2H, J=6.8 Hz), 3.81 (s, 3H), 3.70-3.50 (m, 8H), 3.28 (s, 3H), 2.22 (m, 6H), 1.90 (p, 2H, J=6.8 Hz), 1.68 (m, 4H). MS (ESI): Calcd. for C₂₅H₃₅N₇O₄: 497.6, found 497.9 (M)⁺.

### Example 29: Synthesis of 6-methoxy-N-(5-(methoxymethyl)-1H-pyrazol-3-yl)-2-(4-methoxypiperidin-1-yl)-7-(3-(pyrrolidin-1-yl)propoxy)quinazolin-4-amine, (Compound 29)

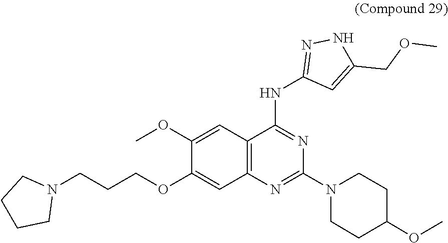 [SMILES: COCc1cc(Nc2nc(N3CCC(OC)CC3)nc3cc(OCCCN4CCCC4)c(OC)cc23)n[nH]1]

Preparation: To a solution of 2-chloro-6-methoxy-N-(5-(methoxymethyl)-1H-pyrazol-3-yl)-7-(3-(pyrrolidin-1-yl)propoxy)quinazolin-4-amine (0.20 g, 0.45 mmol), DIPEA (0.78 mL, 4.47 mmol), and 4-methoxypiperidine (0.21 g, 1.79 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give 6-methoxy-N-(5-(methoxymethyl)-1H-pyrazol-3-yl)-2-(4-methoxypiperidin-1-yl)-7-(3-(pyrrolidin-1-yl)propoxy)quinazolin-4-amine (0.05 g, 23%) as a beige solid. ¹H NMR (400 MHZ, DMSO-d₆): δ 12.44 (bs, 1H), 9.88 (bs, 1H), 7.78 (s, 1H), 6.79 (s, 1H), 6.66 (s, 1H), 4.42 (s, 2H), 4.10 (t, 2H, J=6.8 Hz), 3.80 (s, 3H), 3.79 (s, 3H), 3.76 (m, 4H), 3.33-3.14 (m, 4H), 2.60 (m, 4H), 1.97 (p, 2H, J=6.8 Hz), 1.82 (m, 4H), 1.74 (m, 4H). MS (ESI): Calcd. for C₂₇H₃₉N₇O₄: 525.7, found 525.8 (M)⁺.

### Example 30: Synthesis of 2-(4,4-difluoropiperidin-1-yl)-6-methoxy-AV-(5-(methoxymethyl)-1H-pyrazol-3-yl)-7-(3-(pyrrolidin-1-yl)propoxy)quinazolin-4-amine, (Compound 30)

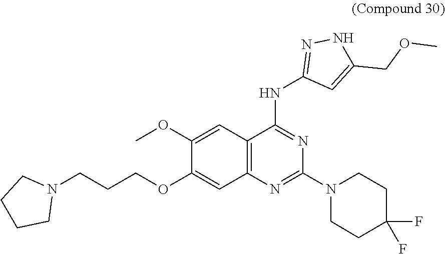 [SMILES: COCc1cc(Nc2nc(N3CCC(F)(F)CC3)nc3cc(OCCCN4CCCC4)c(OC)cc23)n[nH]1]

Preparation: To a solution of 2-chloro-6-methoxy-N-(5-(methoxymethyl)-1H-pyrazol-3-yl)-7-(3-(pyrrolidin-1-yl)propoxy)quinazolin-4-amine (0.20 g, 0.45 mmol), DIPEA (0.78 mL, 4.47 mmol), and 4-methoxypiperidine (0.21 g, 1.79 mmol) in anhydrous THF (6 mL)/2-butanol (2 mL) mixtures under argon. The tube was then sealed and heated to 90° C. for 4 days. The cooled reaction was quenched with sat. NaHCO₃ (2 mL) and then extracted with 8:2 dichloromethane/isopropanol mixtures (3×50 mL). The combined organic layers were dried over anhydrous Na₂SO₄, filtered, and concentrated in vacuo. The residue was purified by Buchi Pureflash chromatography over silica gel cartridge (40 g) with 95:5 CH₂Cl₂:MeOH w/2% 7N ammonia to give 2-(4,4-difluoropiperidin-1-yl)-6-methoxy-N-(5-(methoxymethyl)-1H-pyrazol-3-yl)-7-(3-(pyrrolidin-1-yl)propoxy)quinazolin-4-amine (0.02 g, 7%) as a beige solid. ¹H NMR (400 MHZ, DMSO-d₆): δ 12.49 (bs, 1H), 9.98 (bs, 1H), 7.84 (s, 1H), 7.06 (s, 1H), 6.63 (s, 1H), 4.44 (s, 2H), 4.15 (t, 2H, J=6.8 Hz), 3.86 (s, 3H), 3.03 (m, 2H), 2.28-2.06 (m, 2H), 1.96 (m, 2H), 1.89 (m, 6H), 1.23 (m 4H). MS (ESI): Calcd. for C₂₆H₃₅F₂N₇O₃: 525.7, found 525.8 (M)⁺

The other quinazolinyl compounds disclosed herein may be synthesized in similar fashion to that for Compounds 1-30.

### Example 31: Kinase Inhibition Assays

The activity of Compounds 1, 3, 4, 6, 7, 8, 9, 10 and 13 in inhibiting CLK1, CLK4, PLK4, and FLT3 kinases was determined. Stock solutions of the compounds being evaluated were prepared. The IC₅₀ values were determined by the Eurofins DiscoverX services using either the KinaseProfiler or the KdElect kinase activity detection assays. The results are shown in Table 1. Compound 1 is a selective CLK1/4 inhibitor.

TABLE 1

| | CLK1 | CLK4 | PLK4 | FLT3 |
|---|---|---|---|---|
| Compound | (IC₅₀ nM) | (IC₅₀ nM) | (IC₅₀ nM) | (K_{d50} nM) |
| 1 | +++ | + | − | − |
| 3 | +++ | +++ | +++ | ++ |
| 4 | ++ | + | − | + |
| 6 | +++ | +++ | +++ | +++ |
| 7 | +++ | +++ | − | − |
| 8 | +++ | +++ | +++ | +++ |
| 9 | +++ | +++ | − | − |
| 10 | +++ | +++ | ++ | +++ |
| 13 | +++ | +++ | − | ++ |

+++: IC₅₀: <1 uM
++: IC₅₀: 1-5 uM
+: IC₅₀: 5-10 uM
−: IC₅₀: >10 uM

### Example 32: Cancer Cytotoxicity Assays

The cytotoxicity of Compounds 1, 3, 4, 6, 7, 8, 9, 10, 12 and 13 against cancer cell lines was determined. To determine the optimal plating density for cell viability assays, for each cell line, cells were counted and diluted to final densities of 2.5, 5, 10 and 20 thousand per 100 μL of their respective growth media per well in 96 well plates. The Promega Real-Time-Glo cell viability kit was used to determine the optimal concentration for cell plating so that each cell line would be within the linear part of its growth curve after 72 hrs in culture. For the determination of test compound IC50s, different cancer cell lines were plated in 96 well plates in the pre-determined optimal density for each cell line. Test compounds were then added at different concentrations for the creation of six-point curve and cell density was determined using the Promega Real-Time-Glo cell viability kit after 24, 48, and 72 hrs in culture. Test compound 72 hr IC50s were calculated using the AAT Bioquest IC₅₀ calculator.
+++: IC₅₀: <1 uM
++: IC₅₀: 1-5 uM
+: IC₅₀: 5-10 uM
−: IC₅₀: >10 uM

### Colorectal Cancer Cytotoxicity

The results of cell proliferation assays of Compounds 1, 3, 4, 6, 7, 8, 9, 10, 12 and 13 against the colorectal cancer cell lines HCT-116 and HT-29 demonstrate that the compounds exhibit significant capability for colorectal cancer cell growth inhibition after 72 hours of treatment. The results are shown in Table 2.

TABLE 2

| | HCT-116 | HT-29 |
|---|---|---|
| Compound | (IC₅₀ nM) | (IC₅₀ nM) |
| 1 | ++ | + |
| 3 | ++ | + |
| 4 | ++ | − |
| 6 | ++ | ++ |
| 7 | ++ | ++ |
| 8 | ++ | ++ |
| 9 | + | − |
| 10 | ++ | ++ |
| 12 | ++ | ++ |
| 13 | ++ | ++ |

### Kidney Cancer Cytotoxicity

The results of cell proliferation assays of Compounds 1, 3, 4, 6, 7, 8, 9, 10, 12 and 13 against the colorectal cancer cell lines A-498 and 786-O demonstrate that the compounds exhibit significant capability for kidney cancer cell growth inhibition after 72 hours of treatment. The results are shown in Table 3.

TABLE 3

| | A-498 | 786-O |
|---|---|---|
| Compound | (IC₅₀ nM) | (IC₅₀ nM) |
| 1 | ++ | + |
| 3 | + | + |
| 4 | − | − |
| 6 | ++ | ++ |
| 7 | ++ | ++ |
| 8 | ++ | ++ |
| 9 | ++ | ++ |
| 10 | ++ | ++ |
| 12 | ++ | + |
| 13 | ++ | ++ |

### Ovarian Cancer Cytotoxicity

The results of cell proliferation assays of Compounds 1, 3, 4, 6, 7, 8, 9, 10, 12 and 13 against the ovarian cancer cell lines SKOV-3 and OV-90 demonstrate that the compounds exhibit significant capability for colorectal cancer cell growth inhibition after 72 hours of treatment. The results are shown in Table 4.

TABLE 4

| | SKOV-3 | OV-90 |
|---|---|---|
| Compound | (IC₅₀ nM) | (IC₅₀ nM) |
| 1 | ++ | ++ |
| 3 | ++ | ++ |
| 4 | − | − |
| 6 | ++ | ++ |
| 7 | ++ | ++ |
| 8 | ++ | ++ |
| 9 | ++ | + |
| 10 | ++ | ++ |
| 12 | ++ | ++ |
| 13 | ++ | ++ |

### Leukemia Cell Cytotoxicity

The results of cell proliferation assays of Compounds 1, 3, 4, 6, 7, 8, 9, 10, 12 and 13 against the colorectal cancer cell lines HCT-116 and HT-29 demonstrate that the compounds exhibit significant capability for leukemia cell growth inhibition after 72 hours of treatment. The results are shown in Table 5.

TABLE 5

| | Kasumi-1 | Kasumi-3 | MV4-11 |
|---|---|---|---|
| Compound | (IC₅₀ nM) | (IC₅₀ nM) | (IC₅₀ nM) |
| 1 | ++ | + | +++ |
| 3 | − | + | ++ |
| 4 | − | − | + |
| 6 | ++ | ++ | +++ |
| 7 | ++ | ++ | ++ |
| 8 | ++ | ++ | +++ |
| 9 | ++ | ++ | ++ |
| 10 | +++ | +++ | +++ |
| 12 | ++ | ++ | ++ |
| 13 | ++ | ++ | +++ |

### Example 33: Reduction of Colorectal Tumor Mass

Male nude mice ages 6-8 weeks (NU/J. Jackson Laboratories) received rear, hind leg flank subcutaneous injections of 5×106 HT-29 cells diluted 1:1 in matrigel-cell culture media. Mice were then monitored daily until tumor nodules reached 100-200 mm³ upon which point they were randomized into treatment groups based on their weight and tumor size and treatment. Mice (10 mice per group) were injected I.P. every 48 h with 100 μL of either vehicle control or Compound 8 (5 mg/kg). Tumor nodules were then monitored every 48 h for up to 28 days with tumor volumes calculated using the equation V=(L*W2)/2 where L is length and W is width of a tumor. Differences in xenograft volumes between the different groups were analyzed by single factor analysis of variance of the log-transformed tumor volume data. Mice with tumors that exceed 1000 mm³ were euthanized to avoid excessive suffering.

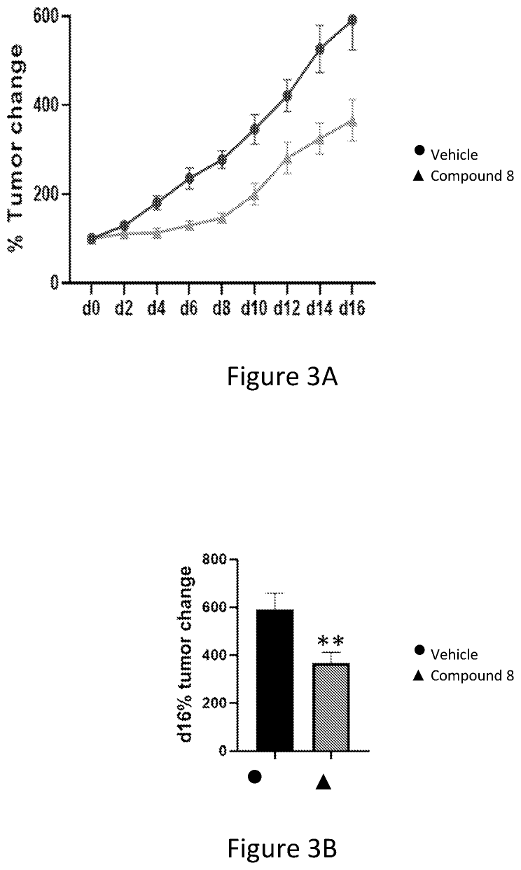

FIG. 3A shows that over the course of a 16 day study test subjects treated with Compound 8 experienced significantly slower tumor growth compared to test subjects treated with the vehicle control. FIG. 3B shows that test subjects treated with the vehicle and with Compound 8 experienced a 600% and 370% increase, respectively. On day 16 the test subjects treated with Compound 8 had significantly smaller tumors compared to test subjects treated with the vehicle.

### Example 34: Prophetic Example of Compounds Having Anti-Cancer Activities

The following compounds are evaluated for their ability to block cancer cell growth in colon (HT-29), liver (HepG2), pancreatic (MiaPaca-2) and bladder (5637) cancer cell lines. Specifically, the Promega Real-Time-Glo cell viability kit was used to determine the optimal concentration for cell plating so that each cell line would be within the linear part of its growth curve after 72 hrs in culture. For the determination of test compound IC₅₀ values, different cancer cell lines are plated in 96 well plates in the pre-determined optimal density for each cell line. Test compounds are then added at different concentrations for the creation of six-point curve and cell density is determined using the Promega Real-Time-Glo cell viability kit after 24, 48, and 72 hrs in culture. The test compound 72 hr IC₅₀ value is calculated using the AAT Bioquest IC₅₀ calculator. Furthermore, the following scoring system is used to display the results in Table 3:

TABLE 6

Cell lines

| Compound | HT-29 | HepG2 | MiaPaca-2 | 5637 |
|---|---|---|---|---|
| 1 | +++ | ++ | − | +++ |
| 3 | ++ | + | ++ | +++ |
| 4 | +++ | − | ++ | +++ |
| 6 | + | +++ | + | ++ |
| 7 | +++ | +++ | − | + |
| 8 | ++ | − | + | +++ |
| 9 | +++ | + | ++ | − |
| 10 | + | + | +++ | ++ |
| 12 | − | ++ | +++ | − |
| 13 | ++ | + | ++ | +++ |
| 14 | +++ | ++ | − | + |
| 18 | ++ | + | +++ | ++ |
| 19 | + | +++ | + | − |
| 20 | − | ++ | +++ | +++ |
| 21 | ++ | + | +++ | ++ |
| 22 | − | +++ | + | − |
| 23 | − | + | +++ | + |
| 24 | ++ | +++ | + | − |
| 25 | +++ | ++ | + | +++ |
| 26 | + | ++ | +++ | + |
| 27 | ++ | − | ++ | + |
| 28 | +++ | − | +++ | ++ |
| 29 | + | ++ | + | + |
| 30 | ++ | + | − | +++ |

+++: IC₅₀: <1 uM
++: IC₅₀: 1-5 uM
+: IC₅₀: 5-10 uM
−: IC₅₀: >10 uM

### Example 35: Treatment of Colon Cancer

Based on the inventor's clinical experience, the following results are projected using controlled studies.

Colon cancer is a type of cancer that begins in the large intestine (colon). The colon is the final part of the digestive tract. Colon cancer typically affects older adults, though it can happen at any age. It usually begins as small, noncancerous (benign) clumps of cells called polyps that form on the inside of the colon. Over time some of these polyps can become colon cancers. A cohort of 90 patients with colon tumors between the ages of 50 and 75 years of age is identified by an oncologist. A detailed examination report for each patent is prepared, complete with an indication of symptoms and their severity. Tumor size is measured using MRI imaging. Symptoms common to the patients include diarrhea and/or constipation, abdominal pain and cramping, rectal pain, rectal bleeding, weight loss, and fatigue. A colonoscopy is also performed to view the tumor(s) in the patient. This report establishes a patient baseline. The experimental group patients (n=30; “EXPT1”) receive Compound 1 once a day orally. The experimental group patients (n=30; “EXPT2) receive Compound 27 once a day orally. The control group patients (n=30; “CONT”) receive a placebo once a day orally. The study is conducted over a period of three months after which patient outcomes are measured by an oncologist. Patients receiving the EXPT1 report improvement in each symptom of colon cancer. They also experience a tumor size reduction on average of 80%. Alternatively, patients in EXPT2 or the CONT group show no decrease in symptoms and/or an increase in symptoms over the course of the study. Tumor size increases over the course of the study. The differences between the EXPT2 and CONT group is not significantly significant. The difference between improved results in the EXPT1 group versus the EXPT2 or CONT group is statistically significant.

### Example 36: Treatment of Pancreatic Cancer

Based on the inventor's clinical experience, the following results are projected using controlled studies.

Pancreatic cancer is a type of cancer that begins in the pancreas which is an organ that releases enzymes that aid digestion and produces hormones for managing blood sugar. Pancreatic cancer typically affects older adults, though it can happen at any age. Pancreatic ductal adenocarcinoma is the most common type and begins in the cells that line the ducts that carry digestive enzymes out of the pancreas. Pancreatic cancer is most treatable at an early stage but is seldom detected until it has spread to other organs and caused noticeable symptoms. A cohort of 90 patients with pancreatic tumors between the ages of 50 and 75 years of age is identified by an oncologist. A detailed examination report for each patent is prepared, complete with an indication of symptoms and their severity. Tumor size is measured using MRI imaging. Symptoms common to the patients include jaundice, dark-colored urine, abdominal pain radiating to the back, itchy skin, blood clots, and fatigue. This report establishes a patient baseline. The experimental group patients (n=30; “EXPT1”) receive Compound 1 once a day orally. The experimental group patients (n=30; “EXPT2) receive Compound 27 once a day orally. The control group patients (n=30; “CONT”) receive a placebo once a day orally. The study is conducted over a period of three months after which patient outcomes are measured by an oncologist. Patients receiving the EXPT1 report improvement in each symptom of pancreatic cancer. They also experience a tumor size reduction on average of 80%. Alternatively, patients in EXPT2 or the CONT group show no decrease in symptoms and/or an increase in symptoms over the course of the study. Tumor size increases over the course of the study. The differences between the EXPT2 and CONT group is not significantly significant. The difference between improved results in the EXPT1 group versus the EXPT2 or CONT group is statistically significant.

### Example 37: Treatment of Bladder Cancer

Based on the inventor's clinical experience, the following results are projected using controlled studies.

Bladder cancer is a type of cancer that begins in the cells of the bladder that is located in the lower abdomen and that functions to store urine. Pancreatic cancer typically affects older adults, though it can happen at any age. It begins most often in the urothelial cells that line the inside of the bladder. Most bladder cancers are diagnosed at an early stage when it is most treatable, but even early-stage bladder cancers can come back after successful treatment. A cohort of 90 patients with bladder cancer between the ages of 50 and 75 years of age is identified by an oncologist. A detailed examination report for each patent is prepared, complete with an indication of symptoms and their severity. Symptoms common to the patients include blood in urine (hematuria), frequent and/or painful urination, and back pain. This report establishes a patient baseline. The experimental group patients (n=30; “EXPT1”) receive Compound 1 once a day orally. The experimental group patients (n=30; “EXPT2) receive Compound 27 once a day orally. The control group patients (n=30; “CONT”) receive a placebo once a day orally. The study is conducted over a period of three months after which patient outcomes are measured by an oncologist. Patients receiving the EXPT1 report improvement in each symptom of pancreatic cancer. Alternatively, patients in EXPT2 or the CONT group show no decrease in symptoms and/or an increase in symptoms over the course of the study. The differences between the EXPT2 and CONT group is not significantly significant. The difference between improved results in the EXPT1 group versus the EXPT2 or CONT group is statistically significant.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the subject matter has been particularly shown and described with reference to a preferred embodiment and various alternate embodiments, it will be understood by persons skilled in the relevant art that various changes in form and details can be made therein without departing from the spirit and scope of the present disclosure.

What is claimed is:

1. A compound of Formula (I): 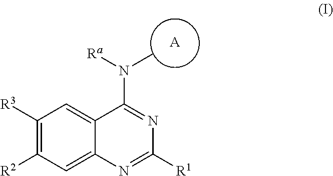
or a stereoisomer, tautomer, or pharmaceutically acceptable salt thereof;
where
R¹ is selected from the group consisting of optionally substituted 6-10 membered aryl, optionally substituted 3-10 membered heterocyclyl, optionally substituted 5-10 membered heteroaryl, optionally substituted carbamide, —CN, and —NR⁴R⁵;
each of R⁴ and R⁵ is independently selected from hydrogen, optionally substituted C₁-C₆ alkyl, or optionally substituted C₃-C₆ carbocyclyl; or alternatively, R⁴ and R⁵ taken together form an optionally substituted 3-10 membered heterocyclyl;
R² is-OR⁶ or optionally substituted (heterocyclyl)alkynyl;
R⁶ is selected from the group consisting of methyl, optionally substituted 2-10 membered heteroalkyl, (carbocyclyl)alkyl, and (heterocyclyl)alkyl; and
R³ is selected from the group consisting of hydrogen, halogen, and C₁₋₆ alkoxy;
R^{a} is hydrogen or optionally substituted C₁-C₁₀ alkyl; and
the A ring is an optionally substituted heteroaryl or an optionally substituted heterocyclyl.

2. The compound of claim 1, wherein the A ring is an optionally substituted heteroaryl having 5 ring members.

3. The compound of claim 1 or 2, wherein the A ring is selected from any of the following: 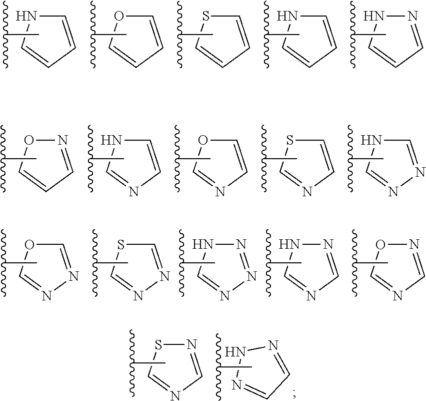
any one of which may be optionally substituted by replacing one or more —H atoms of any carbon or nitrogen atom present on the A ring.

4. The compound of claim 1, wherein the A ring is represented by ring structure (AIa): 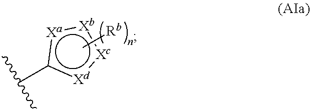
where
each of X^{a}, X^{b}, X^{c}, and X^{d} are independently selected from the group consisting of C, N, O, and S;
any one or more of X^{a}, X^{b}, X^{c}, and X^{d} may be substituted by one or more R^{b} or H groups;
each instance of R^{b}, where present, is independently selected from the group consisting of optionally substituted C₁-C₁₀ alkyl, optionally substituted C₂-C₁₀ alkenyl, and optionally substituted C₃-C₆ carbocyclyl; and
n is an integer selected from 0, 1, 2, 3, or 4.

5. The compound of claim 4, wherein ring structure (AIa) is represented by a structure selected from the group consisting of: 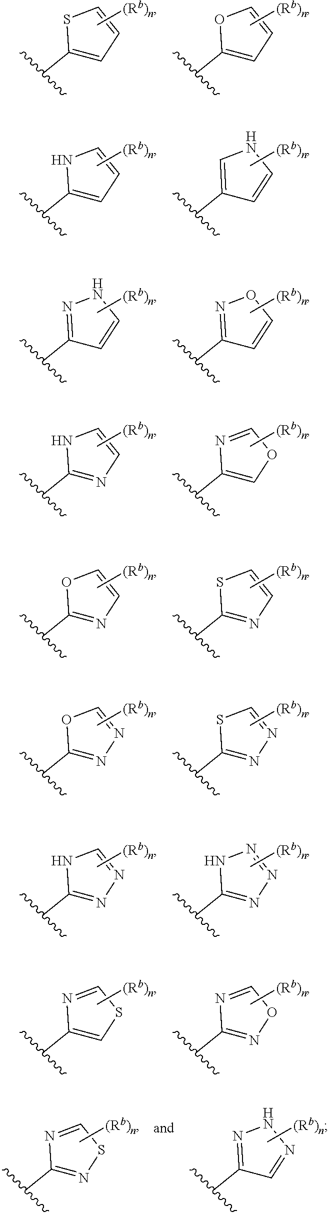
where
each R^{b}, where present, replaces a —H bonded to a C or N atom within ring structure (AIa).

6. The compound of claim 5, wherein ring structure (AIa) is represented by a structure selected from the group consisting of:

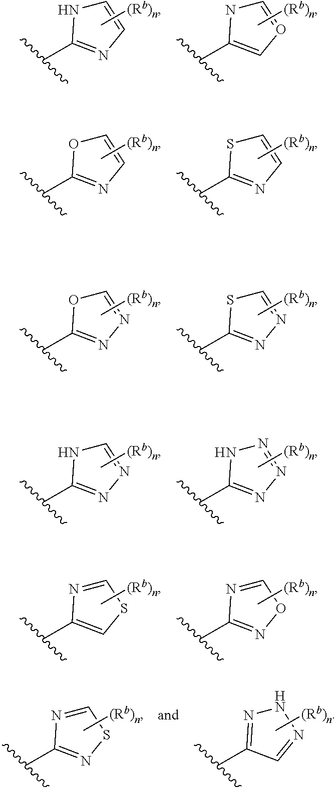

7. The compound of any one of claims 4 to 6, wherein each instance of R^{b}, where present, is selected from the group consisting of: 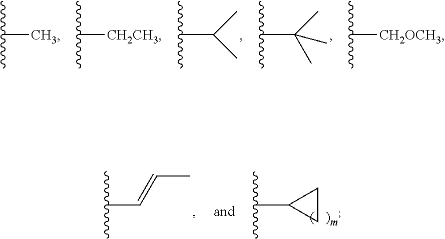
where m is an integer selected from 1, 2, 3, or 4.

8. The compound of claim 7, wherein each instance of R_{D}, where present, is selected from the group consisting of:

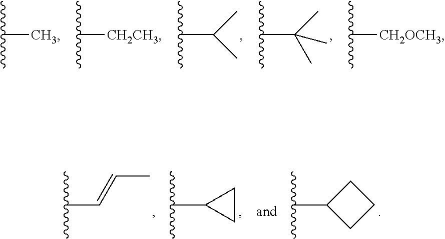

9. The compound of any one of claims 4 to 8, wherein n is 1.

10. The compound of any one of claims 1 to 9, wherein the A ring is a structure selected from the group consisting of:

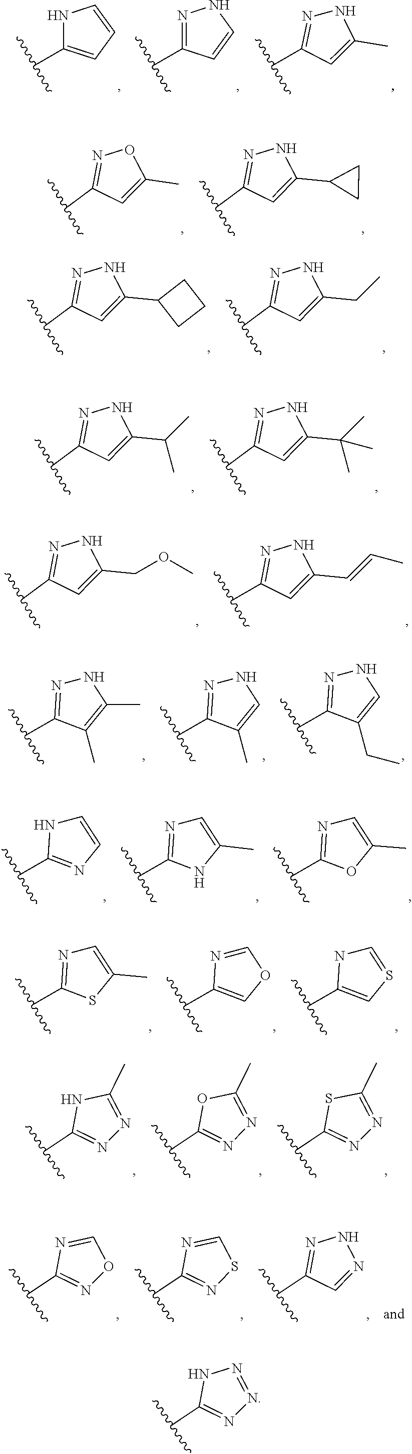

11. The compound of any one of claims 4 to 6, wherein n is 0.

12. The compound of any one of claims 1 to 9, where the A ring is not one of following groups:

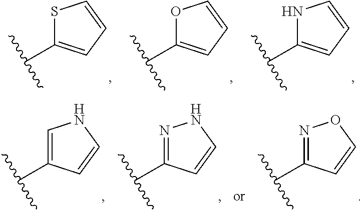 [SMILES: *c1cccs1 ; *c1ccco1 ; *c1ccc[nH]1 ; *c1cc[nH]c1 ; *c1cc[nH]n1 ; *c1ccon1]

13. The compound of any one of claims 1 to 12, where R¹ is a structure selected from the group consisting of:

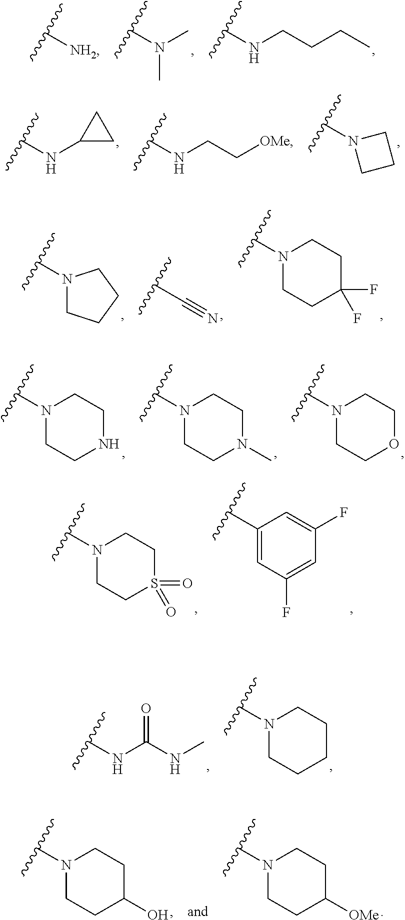

14. The compound of any one of claims 1 to 13, where R² is a (heterocyclyl) C₂-C₆ alkynyl.

15. The compound of any one of claims 1 to 14, where R² is represented by the following structure: 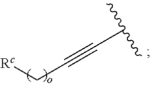
where
R^{c} is a 3 to 8 member heterocyclyl having 1 to 2 heteroatoms; and
o is an integer selected from 1, 2, 3, or 4.

16. The compound of claim 15, where o is 1.

17. The compound of any one of claims 1 to 13, where R² is represented by the following structure: 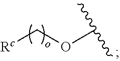
where
R^{c} is a 3 to 8 member heterocyclyl having 1 to 2 heteroatoms or a 2-6 membered heteroalkyl having 1 to 2 heteroatoms; and
o is an integer selected from 1, 2, 3, 4, or 5.

18. The compound of claim 17, where o is 3.

19. The compound of any one of claims 14 to 18, wherein R^{c} has one heteroatom.

20. The compound of any one of claims 14 to 19, wherein R^{c} is the following structure:

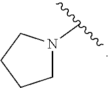 [SMILES: *N1CCCC1]

21. The compound of any one of claims 17 to 19, wherein R^{c} is the following structure:

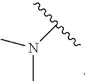 [SMILES: *N(C)C]

22. The compound of any one of claims 1 to 21, where R² is selected from the group consisting of:

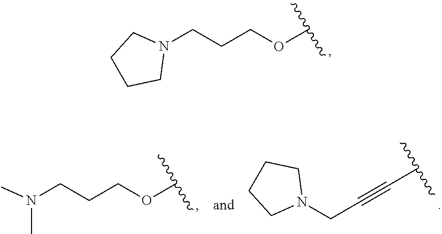

23. The compound of any one of claims 1 to 21, where R³ is —OMe.

24. The compound of any one of claims 1 to 23, where R^{a} is —H.

25. The compound of any one of claims 1 to 23, where R^{a} is methyl.

26. The compound of any one of claims 1 to 24, where, when R^{a} is —H, R³ is —OMe, R¹ is: 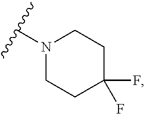 [SMILES: *N1CCC(F)(F)CC1]
and
R² is one of the following structures: 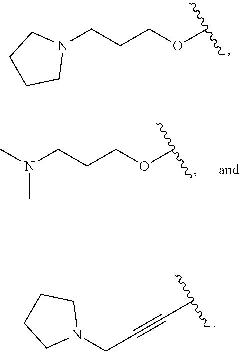
then the A-ring is not the following:

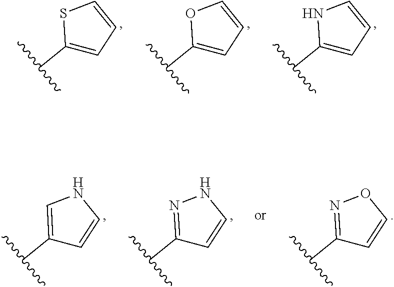 [SMILES: *c1cccs1 ; *c1ccco1 ; *c1ccc[nH]1 ; *c1cc[nH]c1 ; *c1cc[nH]n1 ; *c1ccon1]

27. The compound of any one of claims 1 to 26, where Formula (I) does not include any one of the following structures:

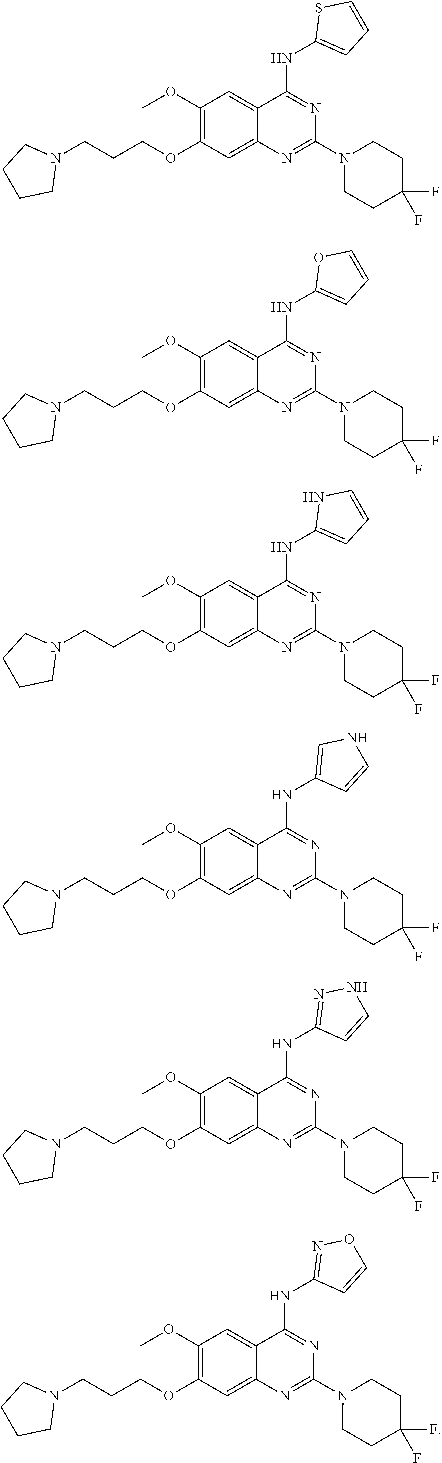

28. The compound of any one of claims 1 to 27, where Formula (I) does not include any one of the following structures:

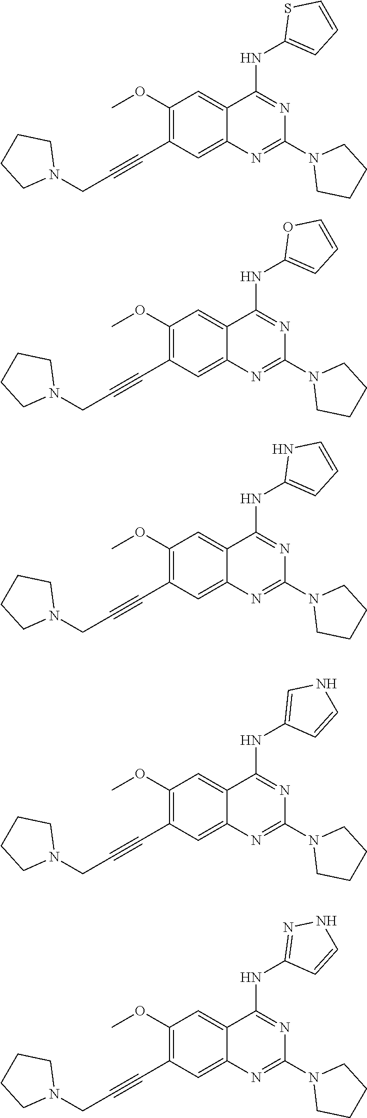

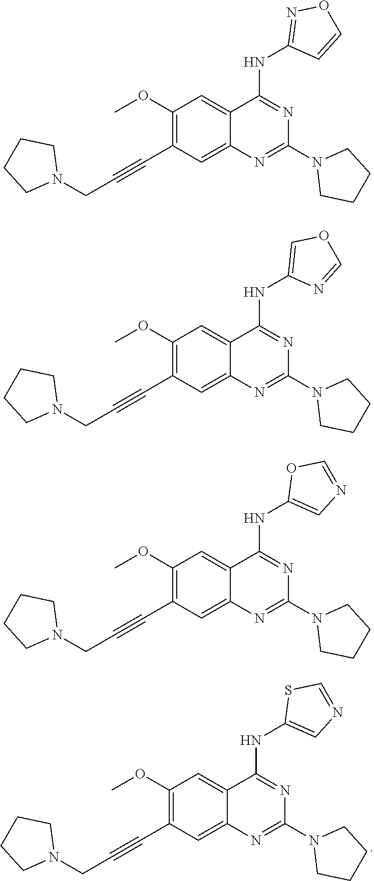

29. The compound of any one of claims 1 to 28, where Formula (I) does not include any one of the following structures:

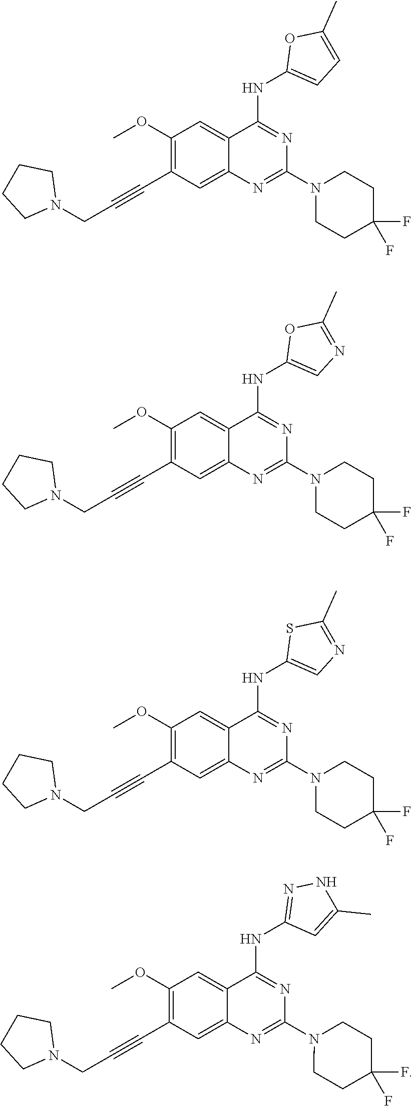 [SMILES: COc1cc2c(Nc3ccc(C)o3)nc(N3CCC(F)(F)CC3)nc2cc1C#CCN1CCCC1 ; COc1cc2c(Nc3cnc(C)o3)nc(N3CCC(F)(F)CC3)nc2cc1C#CCN1CCCC1 ; COc1cc2c(Nc3cnc(C)s3)nc(N3CCC(F)(F)CC3)nc2cc1C#CCN1CCCC1 ; COc1cc2c(Nc3cc(C)[nH]n3)nc(N3CCC(F)(F)CC3)nc2cc1C#CCN1CCCC1]

30. The compound of claim 1, where when R² is: 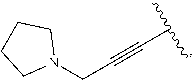 [SMILES: *C#CCN1CCCC1]
then R¹ is not:

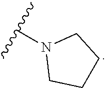 [SMILES: *N1CCCC1]

31. The compound of claim 1, where when R² is: 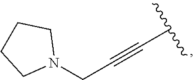 [SMILES: *C#CCN1CCCC1]
then R¹ is:

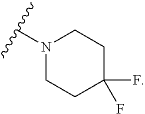 [SMILES: *N1CCC(F)(F)CC1]

32. The compound of claim 1, where the compound of Formula (I) is represented by a compound selected from the group consisting of:

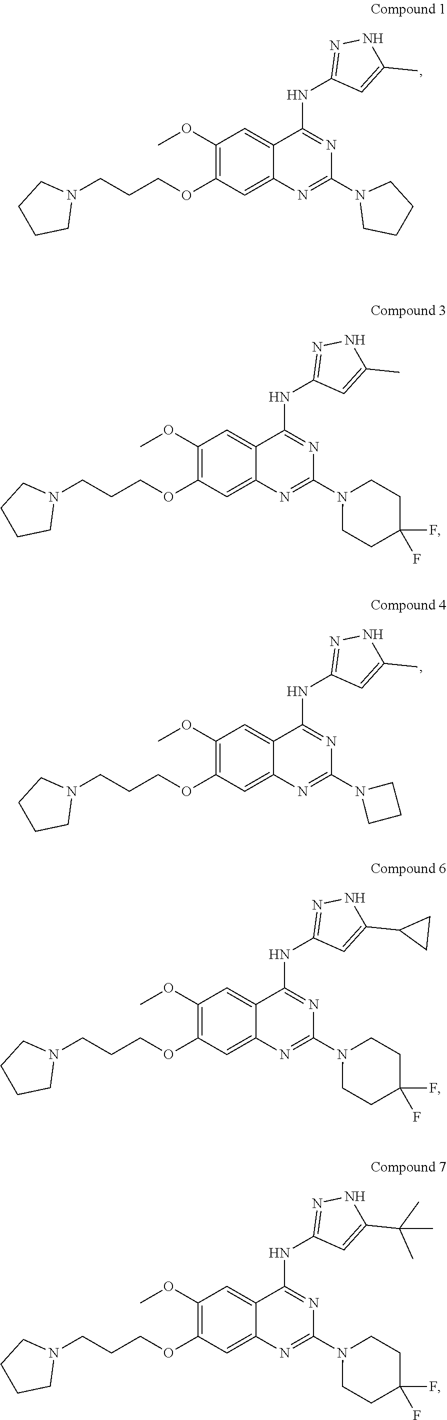 [SMILES: COc1cc2c(Nc3cc(C)[nH]n3)nc(N3CCCC3)nc2cc1OCCCN1CCCC1 ; COc1cc2c(Nc3cc(C)[nH]n3)nc(N3CCC(F)(F)CC3)nc2cc1OCCCN1CCCC1 ; COc1cc2c(Nc3cc(C)[nH]n3)nc(N3CCC3)nc2cc1OCCCN1CCCC1 ; COc1cc2c(Nc3cc(C4CC4)[nH]n3)nc(N3CCC(F)(F)CC3)nc2cc1OCCCN1CCCC1 ; COc1cc2c(Nc3cc(C(C)(C)C)[nH]n3)nc(N3CCC(F)(F)CC3)nc2cc1OCCCN1CCCC1]

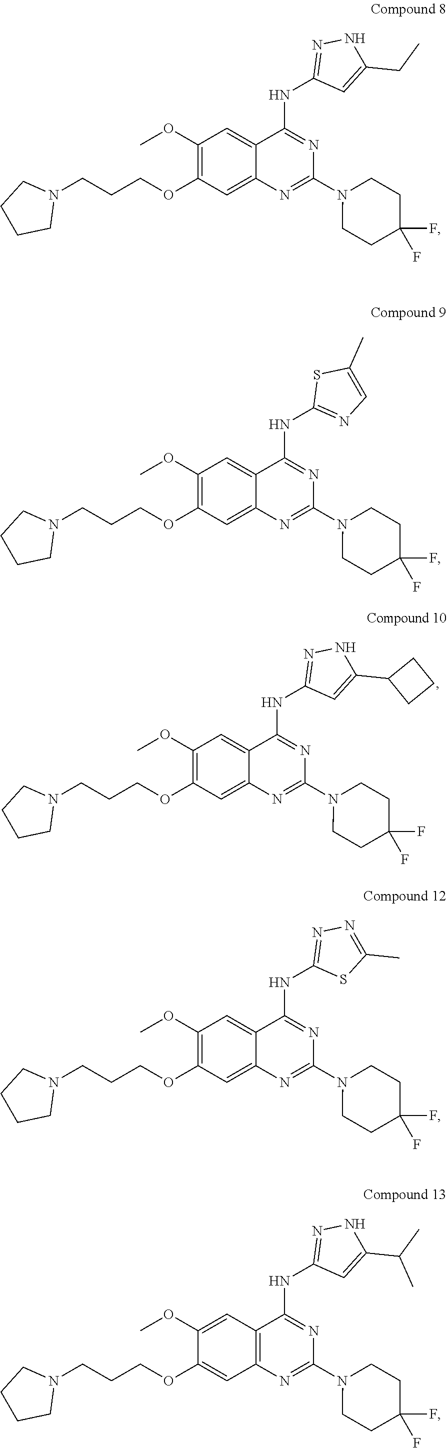 [SMILES: CCc1cc(Nc2nc(N3CCC(F)(F)CC3)nc3cc(OCCCN4CCCC4)c(OC)cc23)n[nH]1 ; COc1cc2c(Nc3ncc(C)s3)nc(N3CCC(F)(F)CC3)nc2cc1OCCCN1CCCC1 ; COc1cc2c(Nc3cc(C4CCC4)[nH]n3)nc(N3CCC(F)(F)CC3)nc2cc1OCCCN1CCCC1 ; COc1cc2c(Nc3nnc(C)s3)nc(N3CCC(F)(F)CC3)nc2cc1OCCCN1CCCC1 ; COc1cc2c(Nc3cc(C(C)C)[nH]n3)nc(N3CCC(F)(F)CC3)nc2cc1OCCCN1CCCC1]

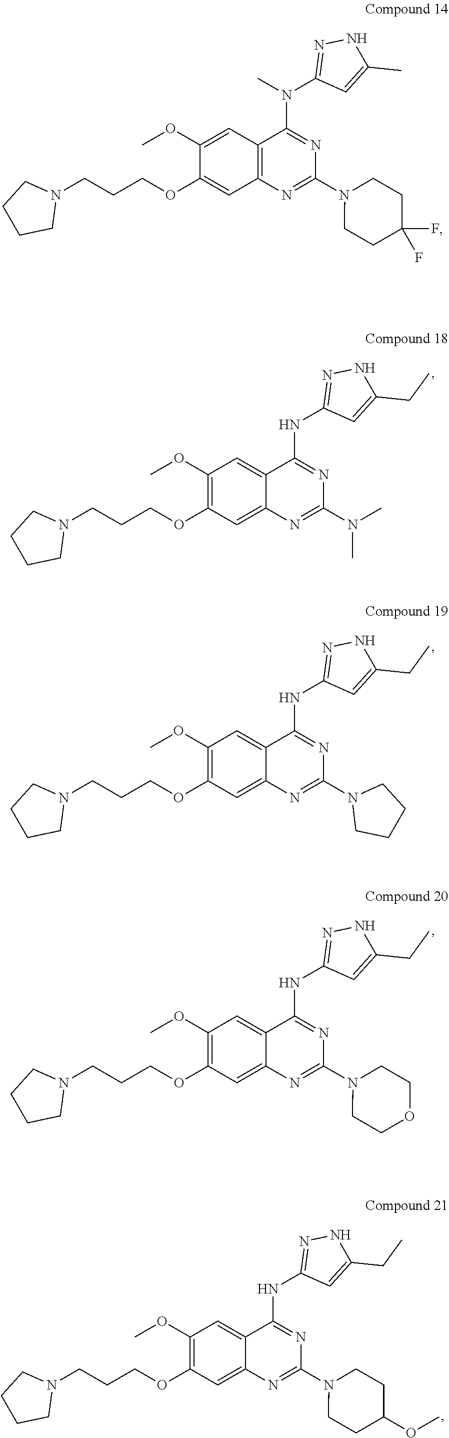 [SMILES: COc1cc2c(N(C)c3cc(C)[nH]n3)nc(N3CCC(F)(F)CC3)nc2cc1OCCCN1CCCC1 ; CCc1cc(Nc2nc(N(C)C)nc3cc(OCCCN4CCCC4)c(OC)cc23)n[nH]1 ; CCc1cc(Nc2nc(N3CCCC3)nc3cc(OCCCN4CCCC4)c(OC)cc23)n[nH]1 ; CCc1cc(Nc2nc(N3CCOCC3)nc3cc(OCCCN4CCCC4)c(OC)cc23)n[nH]1 ; CCc1cc(Nc2nc(N3CCC(OC)CC3)nc3cc(OCCCN4CCCC4)c(OC)cc23)n[nH]1]

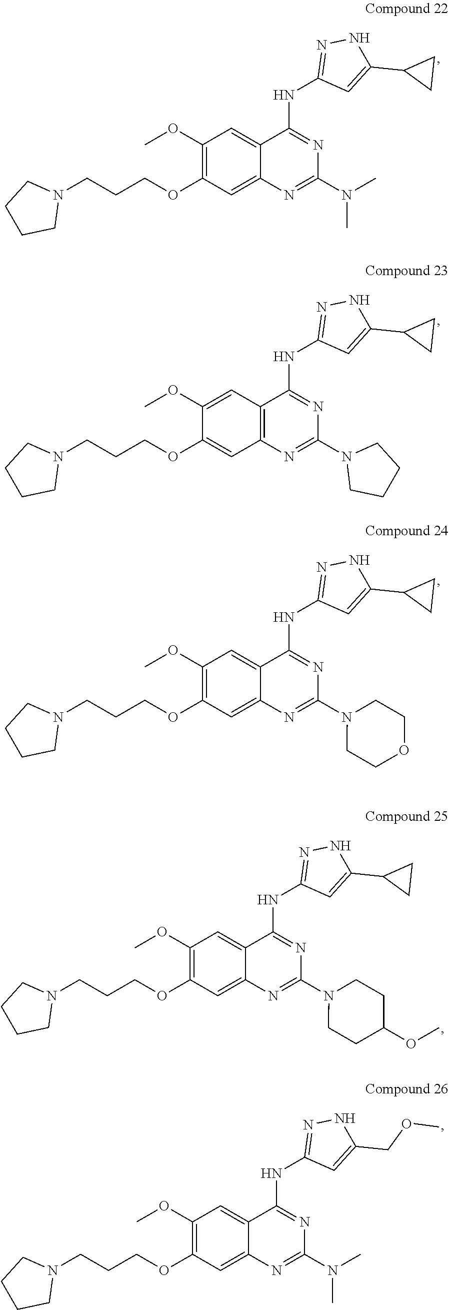 [SMILES: COc1cc2c(Nc3cc(C4CC4)[nH]n3)nc(N(C)C)nc2cc1OCCCN1CCCC1 ; COc1cc2c(Nc3cc(C4CC4)[nH]n3)nc(N3CCCC3)nc2cc1OCCCN1CCCC1 ; COc1cc2c(Nc3cc(C4CC4)[nH]n3)nc(N3CCOCC3)nc2cc1OCCCN1CCCC1 ; COc1cc2c(Nc3cc(C4CC4)[nH]n3)nc(N3CCC(OC)CC3)nc2cc1OCCCN1CCCC1 ; COCc1cc(Nc2nc(N(C)C)nc3cc(OCCCN4CCCC4)c(OC)cc23)n[nH]1]

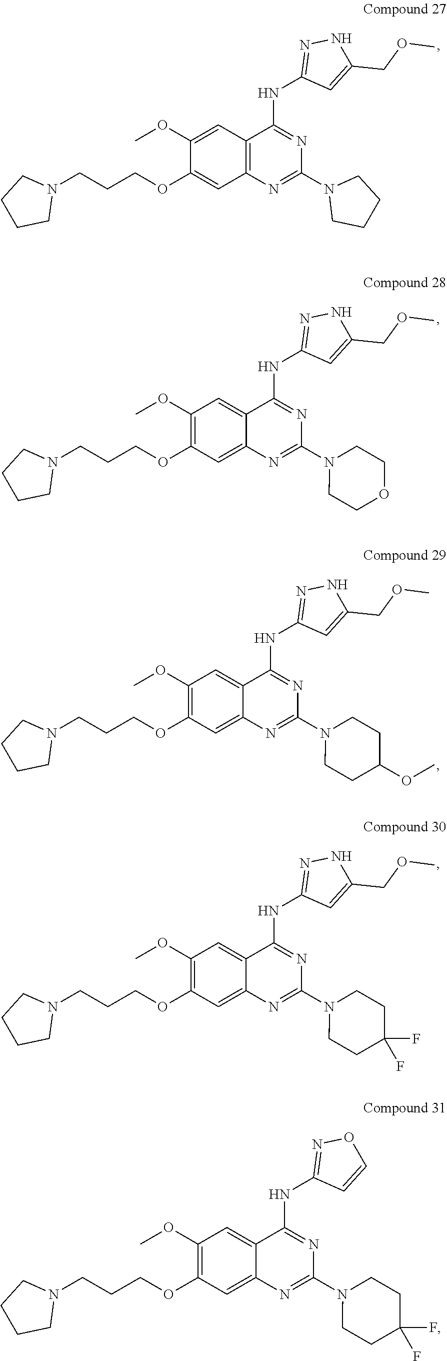 [SMILES: COCc1cc(Nc2nc(N3CCCC3)nc3cc(OCCCN4CCCC4)c(OC)cc23)n[nH]1 ; COCc1cc(Nc2nc(N3CCOCC3)nc3cc(OCCCN4CCCC4)c(OC)cc23)n[nH]1 ; COCc1cc(Nc2nc(N3CCC(OC)CC3)nc3cc(OCCCN4CCCC4)c(OC)cc23)n[nH]1 ; COCc1cc(Nc2nc(N3CCC(F)(F)CC3)nc3cc(OCCCN4CCCC4)c(OC)cc23)n[nH]1 ; COc1cc2c(Nc3ccon3)nc(N3CCC(F)(F)CC3)nc2cc1OCCCN1CCCC1]

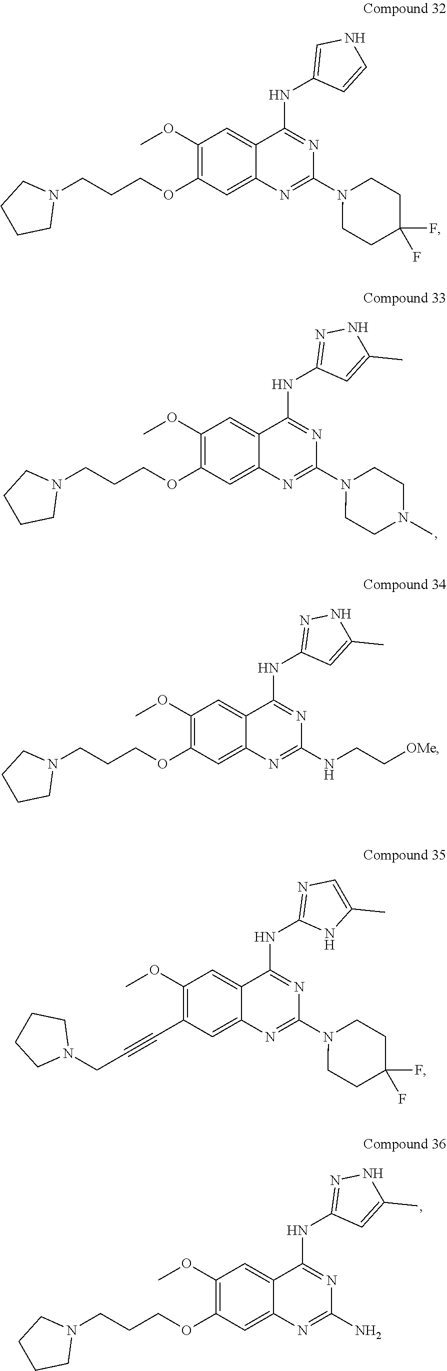 [SMILES: COc1cc2c(Nc3cc[nH]c3)nc(N3CCC(F)(F)CC3)nc2cc1OCCCN1CCCC1 ; COc1cc2c(Nc3cc(C)[nH]n3)nc(N3CCN(C)CC3)nc2cc1OCCCN1CCCC1 ; COCCNc1nc(Nc2cc(C)[nH]n2)c2cc(OC)c(OCCCN3CCCC3)cc2n1 ; COc1cc2c(Nc3ncc(C)[nH]3)nc(N3CCC(F)(F)CC3)nc2cc1C#CCN1CCCC1 ; COc1cc2c(Nc3cc(C)[nH]n3)nc(N)nc2cc1OCCCN1CCCC1]

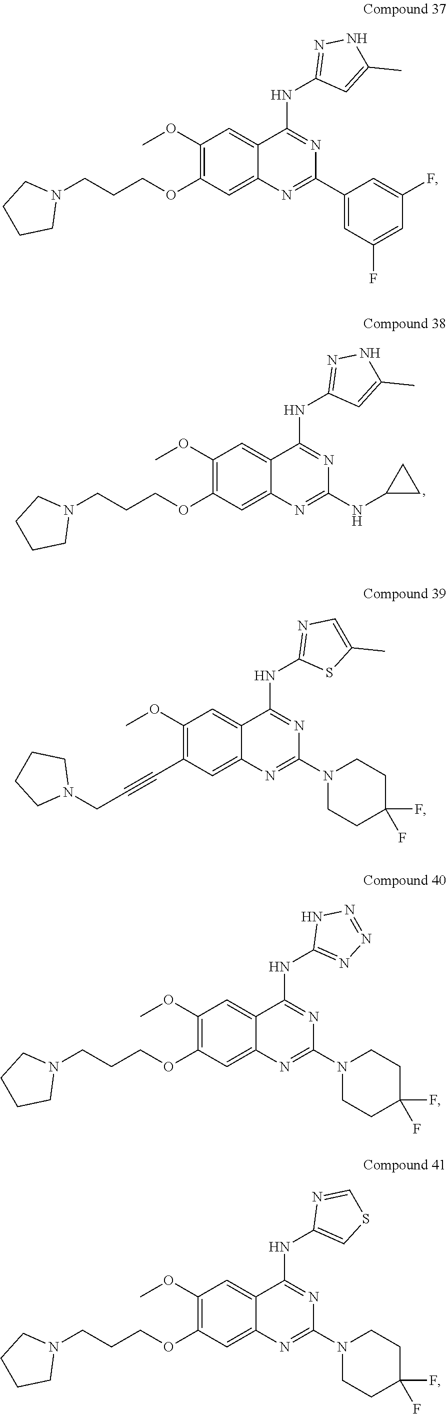 [SMILES: COc1cc2c(Nc3cc(C)[nH]n3)nc(-c3cc(F)cc(F)c3)nc2cc1OCCCN1CCCC1 ; COc1cc2c(Nc3cc(C)[nH]n3)nc(NC3CC3)nc2cc1OCCCN1CCCC1 ; COc1cc2c(Nc3ncc(C)s3)nc(N3CCC(F)(F)CC3)nc2cc1C#CCN1CCCC1 ; COc1cc2c(Nc3nnn[nH]3)nc(N3CCC(F)(F)CC3)nc2cc1OCCCN1CCCC1 ; COc1cc2c(Nc3cscn3)nc(N3CCC(F)(F)CC3)nc2cc1OCCCN1CCCC1]

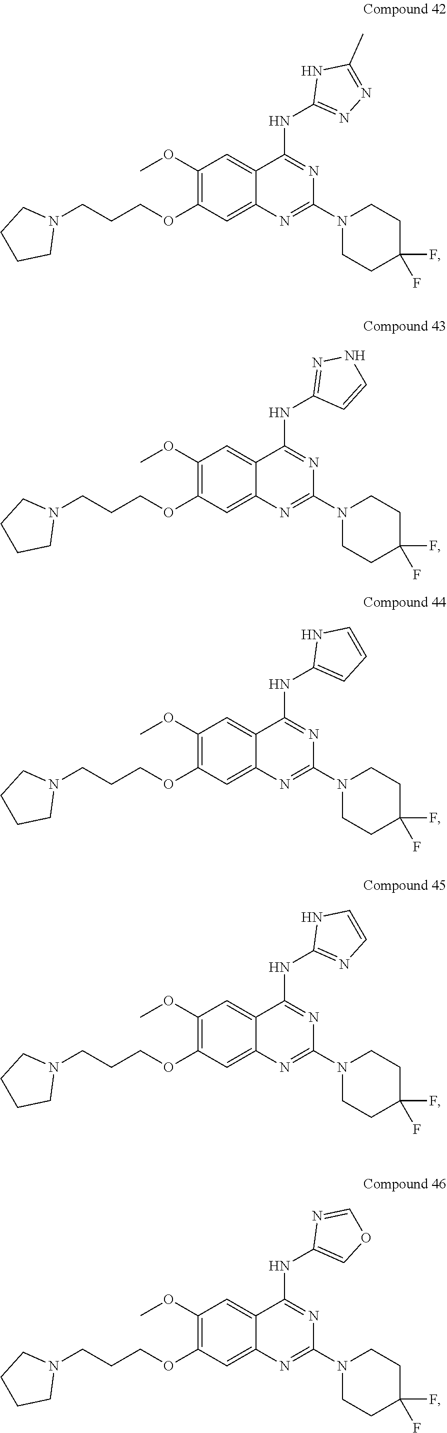 [SMILES: COc1cc2c(Nc3nnc(C)[nH]3)nc(N3CCC(F)(F)CC3)nc2cc1OCCCN1CCCC1 ; COc1cc2c(Nc3cc[nH]n3)nc(N3CCC(F)(F)CC3)nc2cc1OCCCN1CCCC1 ; COc1cc2c(Nc3ccc[nH]3)nc(N3CCC(F)(F)CC3)nc2cc1OCCCN1CCCC1 ; COc1cc2c(Nc3ncc[nH]3)nc(N3CCC(F)(F)CC3)nc2cc1OCCCN1CCCC1 ; COc1cc2c(Nc3cocn3)nc(N3CCC(F)(F)CC3)nc2cc1OCCCN1CCCC1]

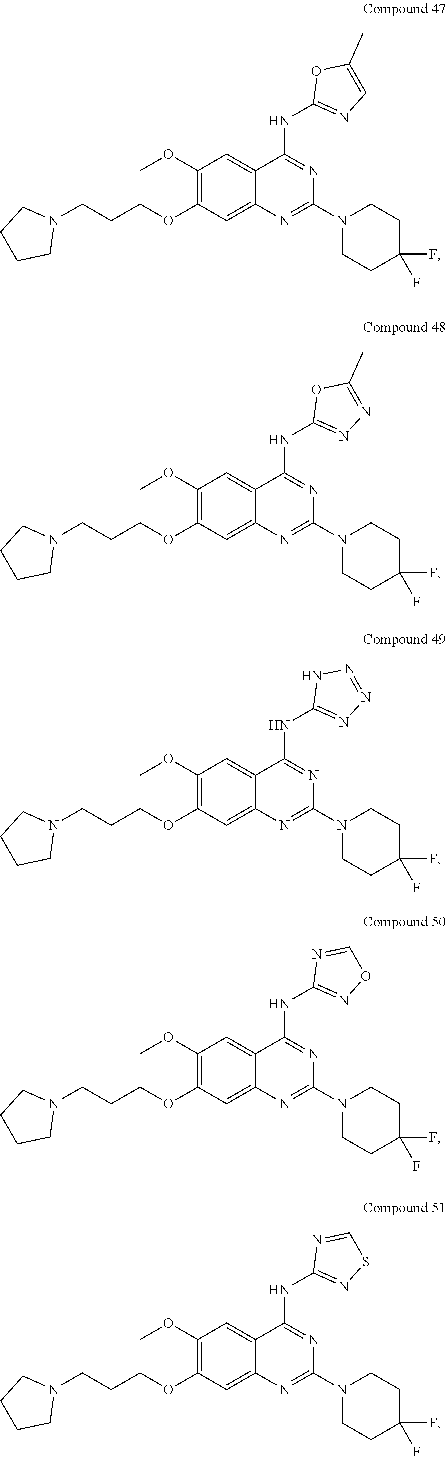 [SMILES: COc1cc2c(Nc3ncc(C)o3)nc(N3CCC(F)(F)CC3)nc2cc1OCCCN1CCCC1 ; COc1cc2c(Nc3nnc(C)o3)nc(N3CCC(F)(F)CC3)nc2cc1OCCCN1CCCC1 ; COc1cc2c(Nc3nnn[nH]3)nc(N3CCC(F)(F)CC3)nc2cc1OCCCN1CCCC1 ; COc1cc2c(Nc3ncon3)nc(N3CCC(F)(F)CC3)nc2cc1OCCCN1CCCC1 ; COc1cc2c(Nc3ncsn3)nc(N3CCC(F)(F)CC3)nc2cc1OCCCN1CCCC1]

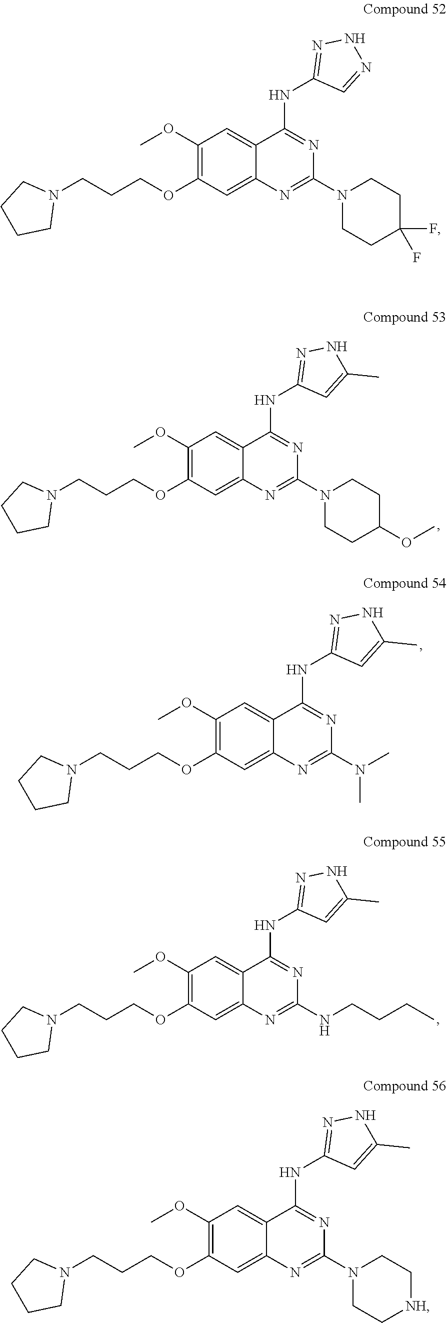 [SMILES: COc1cc2c(Nc3cn[nH]n3)nc(N3CCC(F)(F)CC3)nc2cc1OCCCN1CCCC1 ; COc1cc2c(Nc3cc(C)[nH]n3)nc(N3CCC(OC)CC3)nc2cc1OCCCN1CCCC1 ; COc1cc2c(Nc3cc(C)[nH]n3)nc(N(C)C)nc2cc1OCCCN1CCCC1 ; CCCCNc1nc(Nc2cc(C)[nH]n2)c2cc(OC)c(OCCCN3CCCC3)cc2n1 ; COc1cc2c(Nc3cc(C)[nH]n3)nc(N3CCNCC3)nc2cc1OCCCN1CCCC1]

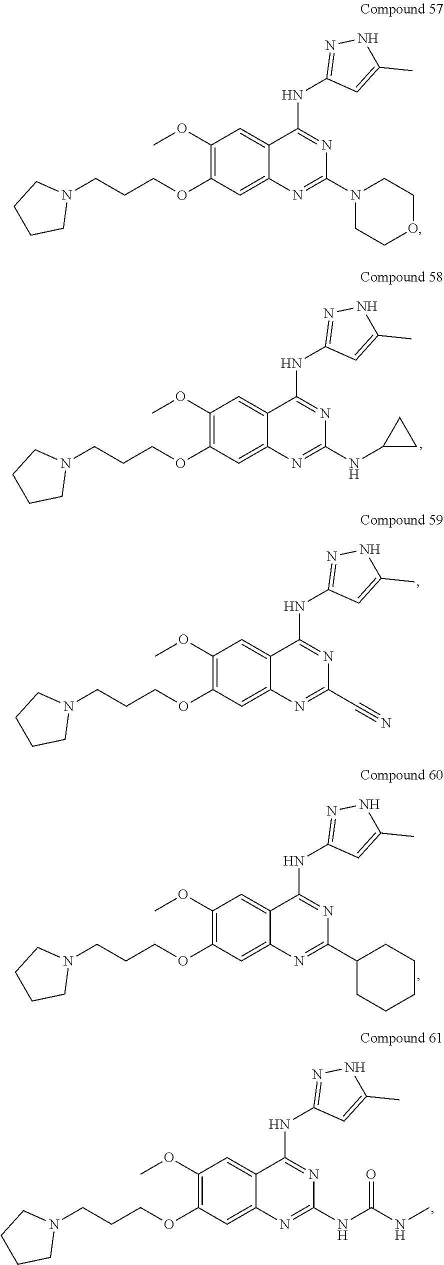 [SMILES: COc1cc2c(Nc3cc(C)[nH]n3)nc(N3CCOCC3)nc2cc1OCCCN1CCCC1 ; COc1cc2c(Nc3cc(C)[nH]n3)nc(NC3CC3)nc2cc1OCCCN1CCCC1 ; COc1cc2c(Nc3cc(C)[nH]n3)nc(C#N)nc2cc1OCCCN1CCCC1 ; COc1cc2c(Nc3cc(C)[nH]n3)nc(C3CCCCC3)nc2cc1OCCCN1CCCC1 ; CNC(=O)Nc1nc(Nc2cc(C)[nH]n2)c2cc(OC)c(OCCCN3CCCC3)cc2n1]

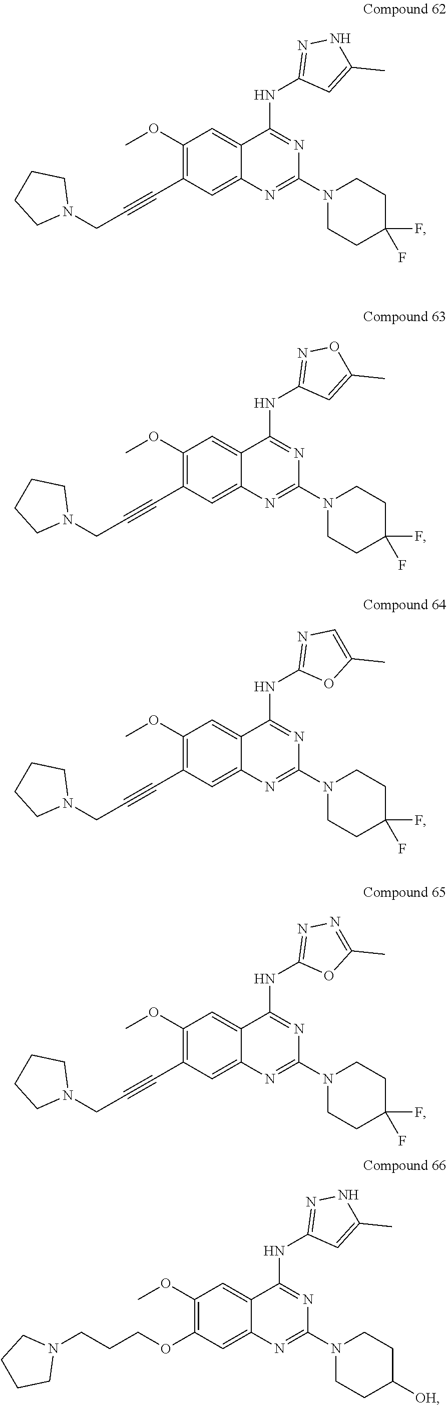 [SMILES: COc1cc2c(Nc3cc(C)[nH]n3)nc(N3CCC(F)(F)CC3)nc2cc1C#CCN1CCCC1 ; COc1cc2c(Nc3cc(C)on3)nc(N3CCC(F)(F)CC3)nc2cc1C#CCN1CCCC1 ; COc1cc2c(Nc3ncc(C)o3)nc(N3CCC(F)(F)CC3)nc2cc1C#CCN1CCCC1 ; COc1cc2c(Nc3nnc(C)o3)nc(N3CCC(F)(F)CC3)nc2cc1C#CCN1CCCC1 ; COc1cc2c(Nc3cc(C)[nH]n3)nc(N3CCC(O)CC3)nc2cc1OCCCN1CCCC1]

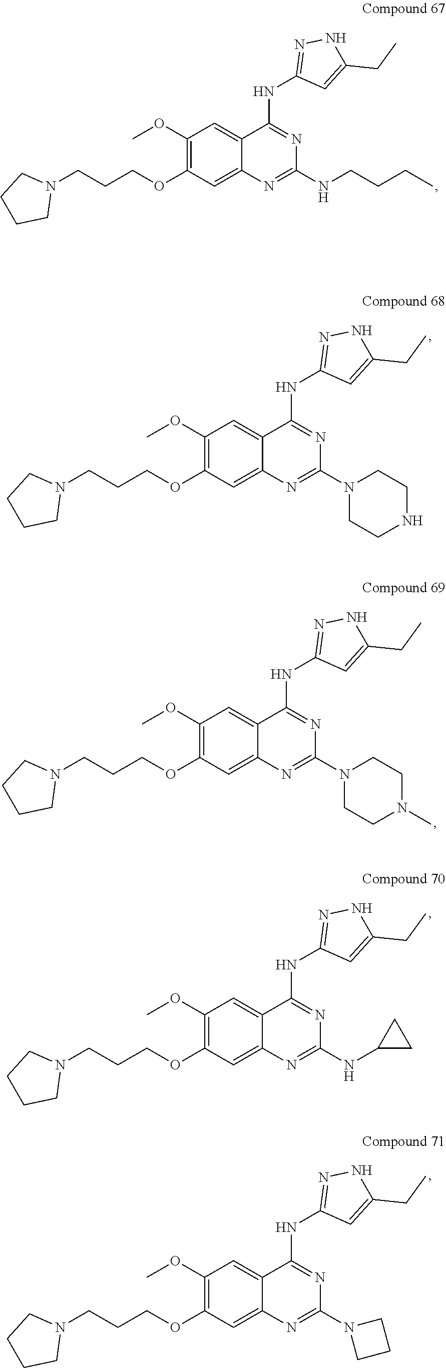 [SMILES: CCCCNc1nc(Nc2cc(CC)[nH]n2)c2cc(OC)c(OCCCN3CCCC3)cc2n1 ; CCc1cc(Nc2nc(N3CCNCC3)nc3cc(OCCCN4CCCC4)c(OC)cc23)n[nH]1 ; CCc1cc(Nc2nc(N3CCN(C)CC3)nc3cc(OCCCN4CCCC4)c(OC)cc23)n[nH]1 ; CCc1cc(Nc2nc(NC3CC3)nc3cc(OCCCN4CCCC4)c(OC)cc23)n[nH]1 ; CCc1cc(Nc2nc(N3CCC3)nc3cc(OCCCN4CCCC4)c(OC)cc23)n[nH]1]

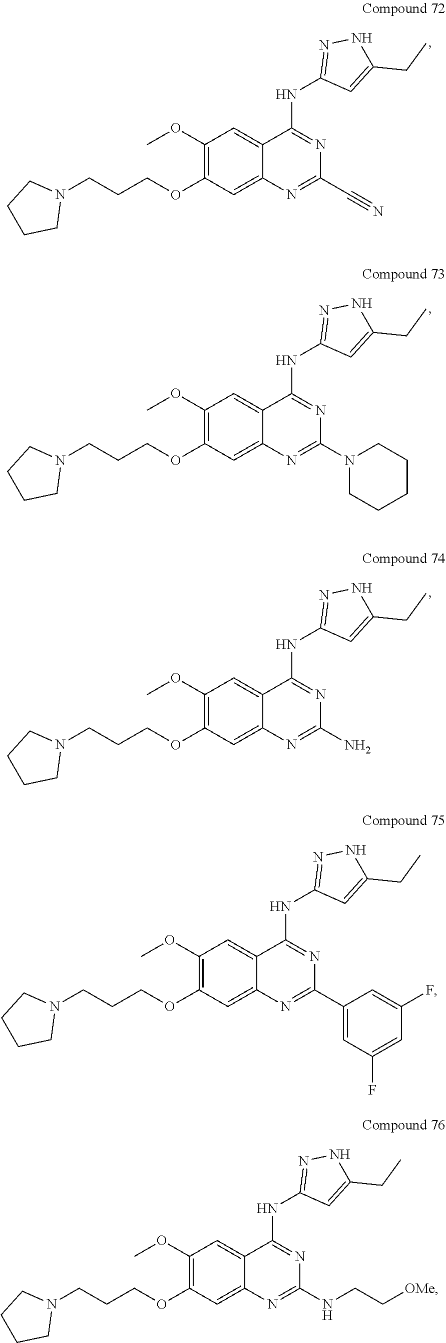 [SMILES: CCc1cc(Nc2nc(C#N)nc3cc(OCCCN4CCCC4)c(OC)cc23)n[nH]1 ; CCc1cc(Nc2nc(N3CCCCC3)nc3cc(OCCCN4CCCC4)c(OC)cc23)n[nH]1 ; CCc1cc(Nc2nc(N)nc3cc(OCCCN4CCCC4)c(OC)cc23)n[nH]1 ; CCc1cc(Nc2nc(-c3cc(F)cc(F)c3)nc3cc(OCCCN4CCCC4)c(OC)cc23)n[nH]1 ; CCc1cc(Nc2nc(NCCOC)nc3cc(OCCCN4CCCC4)c(OC)cc23)n[nH]1]

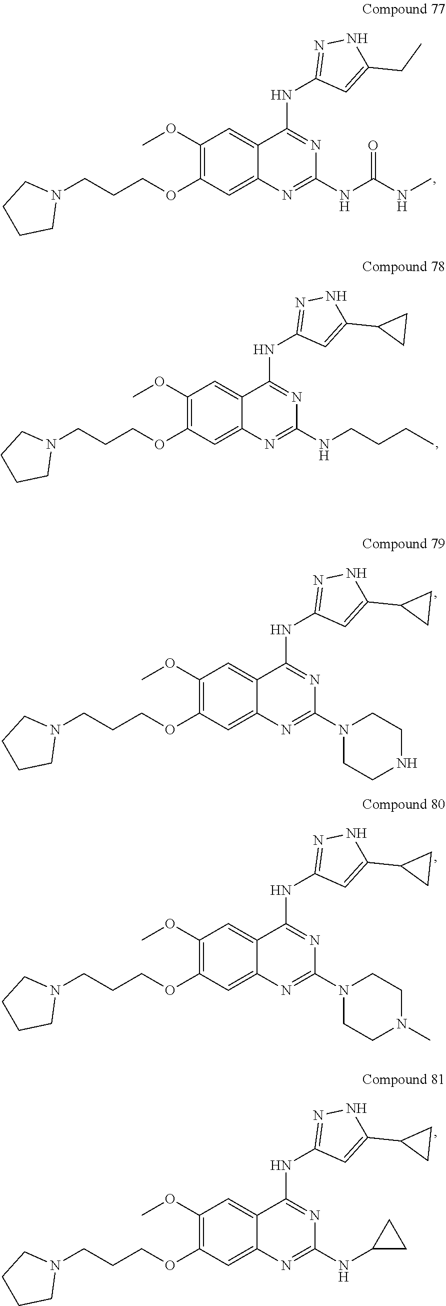 [SMILES: CCc1cc(Nc2nc(NC(=O)NC)nc3cc(OCCCN4CCCC4)c(OC)cc23)n[nH]1 ; CCCCNc1nc(Nc2cc(C3CC3)[nH]n2)c2cc(OC)c(OCCCN3CCCC3)cc2n1 ; COc1cc2c(Nc3cc(C4CC4)[nH]n3)nc(N3CCNCC3)nc2cc1OCCCN1CCCC1 ; COc1cc2c(Nc3cc(C4CC4)[nH]n3)nc(N3CCN(C)CC3)nc2cc1OCCCN1CCCC1 ; COc1cc2c(Nc3cc(C4CC4)[nH]n3)nc(NC3CC3)nc2cc1OCCCN1CCCC1]

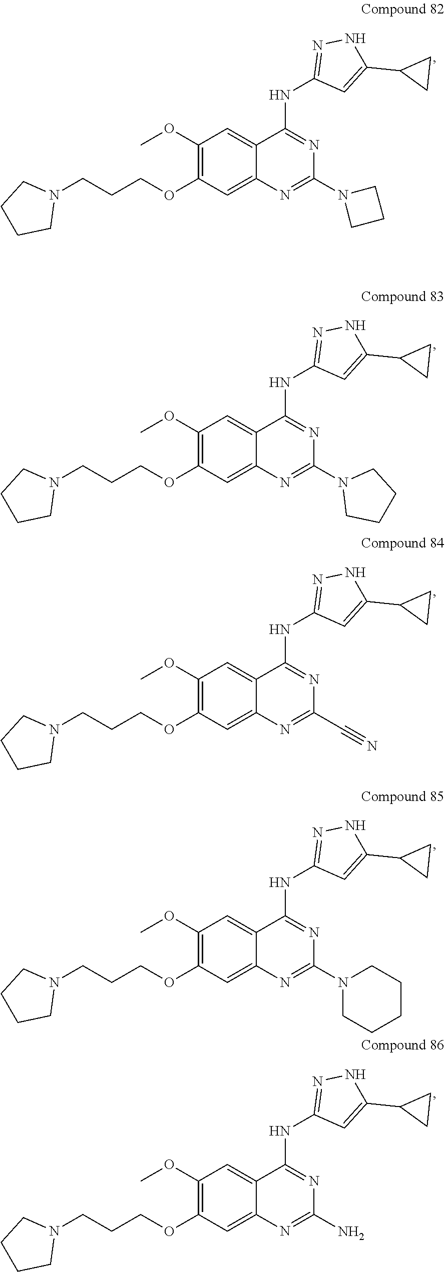 [SMILES: COc1cc2c(Nc3cc(C4CC4)[nH]n3)nc(N3CCC3)nc2cc1OCCCN1CCCC1 ; COc1cc2c(Nc3cc(C4CC4)[nH]n3)nc(N3CCCC3)nc2cc1OCCCN1CCCC1 ; COc1cc2c(Nc3cc(C4CC4)[nH]n3)nc(C#N)nc2cc1OCCCN1CCCC1 ; COc1cc2c(Nc3cc(C4CC4)[nH]n3)nc(N3CCCCC3)nc2cc1OCCCN1CCCC1 ; COc1cc2c(Nc3cc(C4CC4)[nH]n3)nc(N)nc2cc1OCCCN1CCCC1]

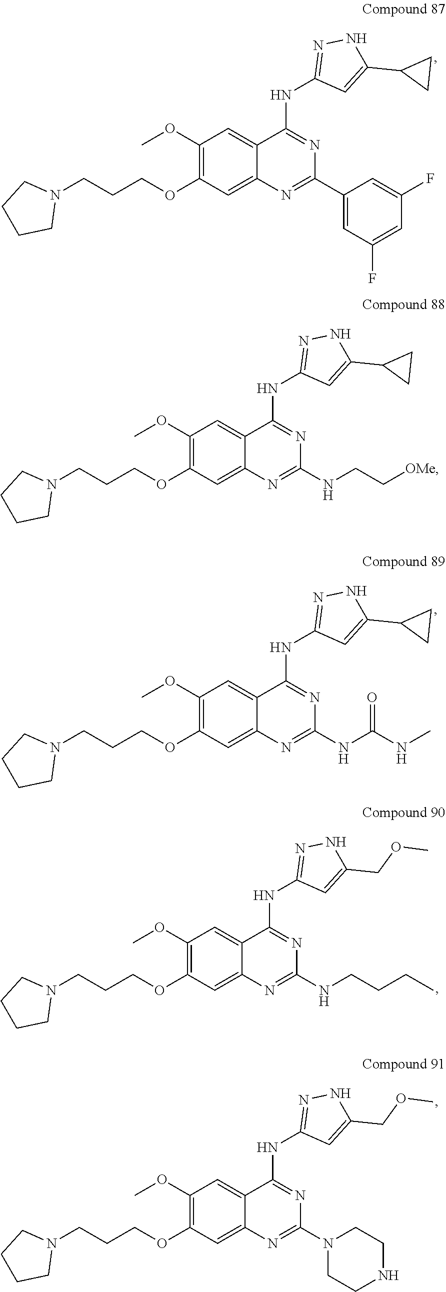 [SMILES: COc1cc2c(Nc3cc(C4CC4)[nH]n3)nc(-c3cc(F)cc(F)c3)nc2cc1OCCCN1CCCC1 ; COCCNc1nc(Nc2cc(C3CC3)[nH]n2)c2cc(OC)c(OCCCN3CCCC3)cc2n1 ; CNC(=O)Nc1nc(Nc2cc(C3CC3)[nH]n2)c2cc(OC)c(OCCCN3CCCC3)cc2n1 ; CCCCNc1nc(Nc2cc(COC)[nH]n2)c2cc(OC)c(OCCCN3CCCC3)cc2n1 ; COCc1cc(Nc2nc(N3CCNCC3)nc3cc(OCCCN4CCCC4)c(OC)cc23)n[nH]1]

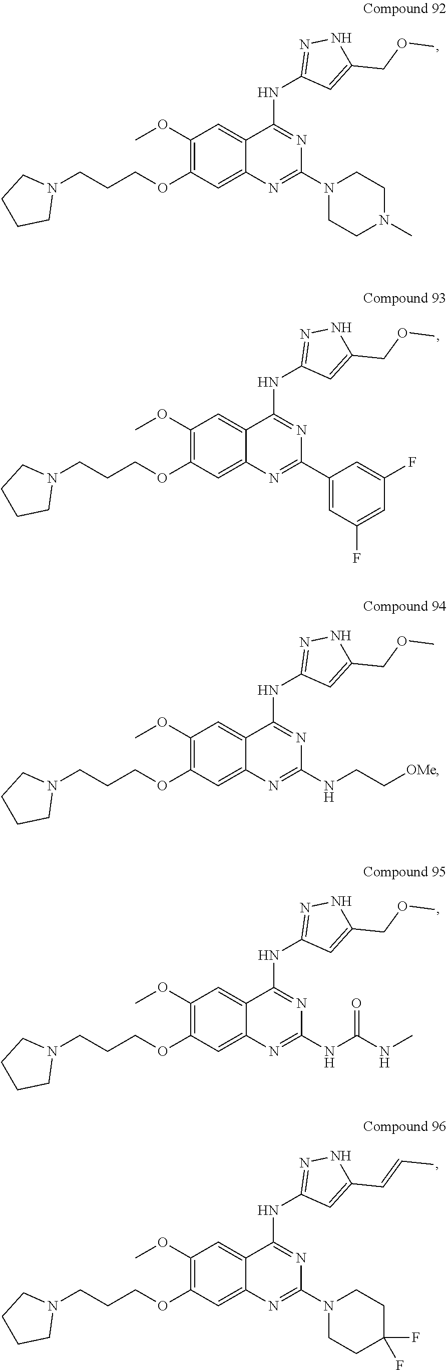 [SMILES: COCc1cc(Nc2nc(N3CCN(C)CC3)nc3cc(OCCCN4CCCC4)c(OC)cc23)n[nH]1 ; COCc1cc(Nc2nc(-c3cc(F)cc(F)c3)nc3cc(OCCCN4CCCC4)c(OC)cc23)n[nH]1 ; COCCNc1nc(Nc2cc(COC)[nH]n2)c2cc(OC)c(OCCCN3CCCC3)cc2n1 ; CNC(=O)Nc1nc(Nc2cc(COC)[nH]n2)c2cc(OC)c(OCCCN3CCCC3)cc2n1 ; C/C=C/c1cc(Nc2nc(N3CCC(F)(F)CC3)nc3cc(OCCCN4CCCC4)c(OC)cc23)n[nH]1]

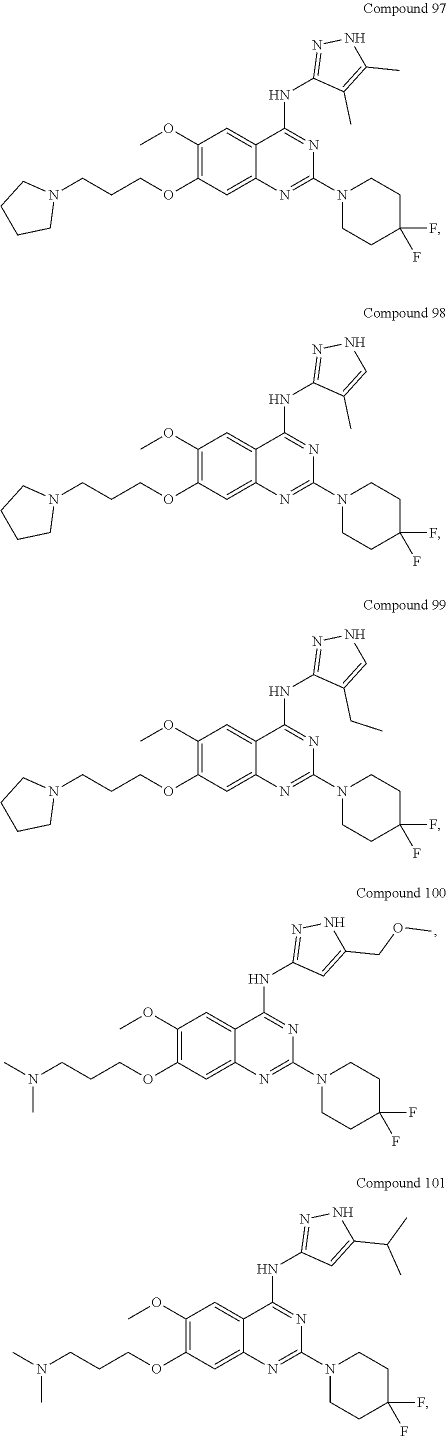

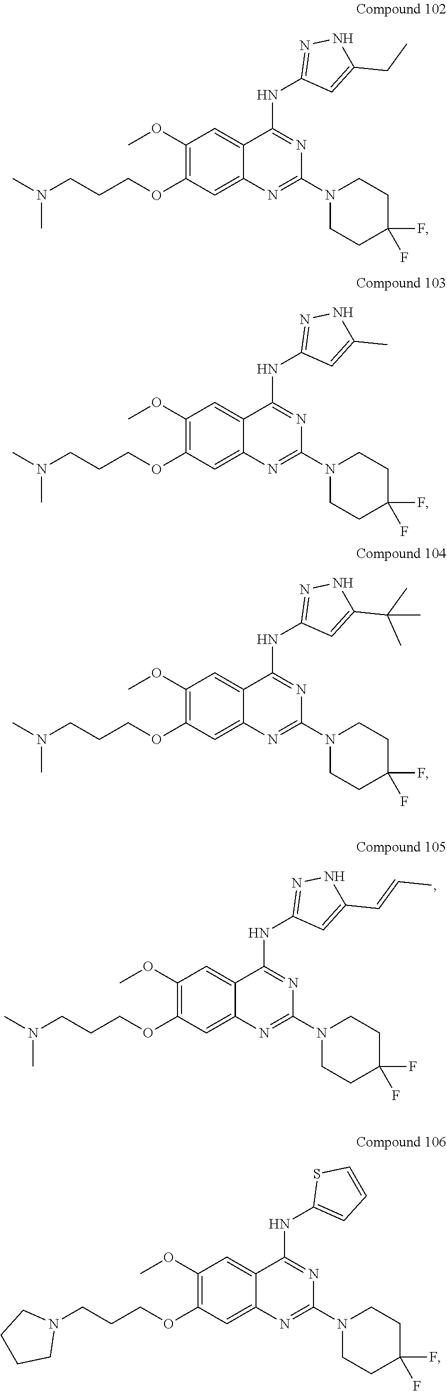 [SMILES: CCc1cc(Nc2nc(N3CCC(F)(F)CC3)nc3cc(OCCCN(C)C)c(OC)cc23)n[nH]1 ; COc1cc2c(Nc3cc(C)[nH]n3)nc(N3CCC(F)(F)CC3)nc2cc1OCCCN(C)C ; COc1cc2c(Nc3cc(C(C)(C)C)[nH]n3)nc(N3CCC(F)(F)CC3)nc2cc1OCCCN(C)C ; C/C=C/c1cc(Nc2nc(N3CCC(F)(F)CC3)nc3cc(OCCCN(C)C)c(OC)cc23)n[nH]1 ; COc1cc2c(Nc3cccs3)nc(N3CCC(F)(F)CC3)nc2cc1OCCCN1CCCC1]

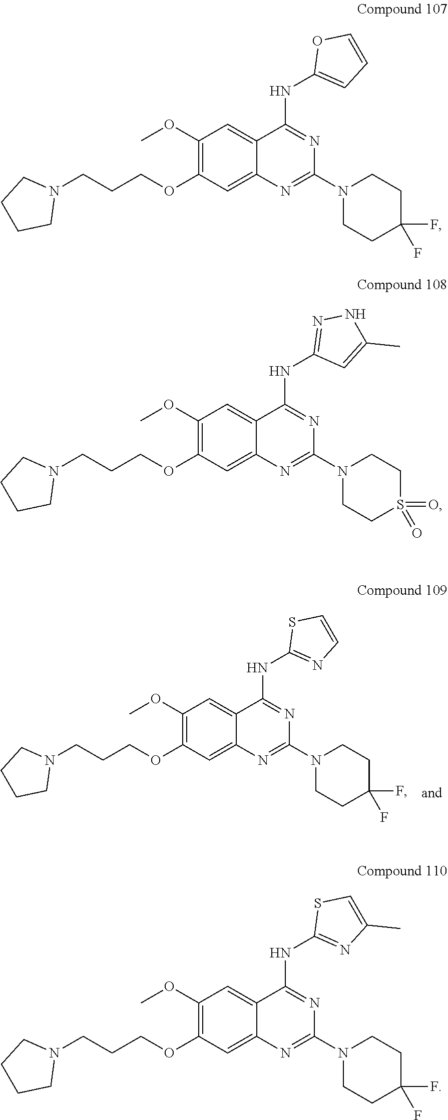 [SMILES: COc1cc2c(Nc3ccco3)nc(N3CCC(F)(F)CC3)nc2cc1OCCCN1CCCC1 ; COc1cc2c(Nc3cc(C)[nH]n3)nc(N3CCS(=O)(=O)CC3)nc2cc1OCCCN1CCCC1 ; COc1cc2c(Nc3nccs3)nc(N3CCC(F)(F)CC3)nc2cc1OCCCN1CCCC1 ; COc1cc2c(Nc3nc(C)cs3)nc(N3CCC(F)(F)CC3)nc2cc1OCCCN1CCCC1]

33. The compound of any one of claims 1 to 32, where, when substituted, the optional substitutions of the R¹ are selected from one or more of amino, —OH, optionally substituted C₁-C₆ alkyl, optionally substituted C₁-C₆ alkoxy, and halogen.

34. The compound of any one of claims 1 to 33, where, when substituted, the optional substitutions of the R² are selected from one or more of amino, —OH, optionally substituted C₁-C₆ alkyl, optionally substituted C₁-C₆ alkoxy, and halogen.

35. The compound of any one of claims 1 to 34, where, when substituted, the optional substitutions of the R^{a} are selected from one or more of amino, —OH, optionally substituted C₁-C₆ alkyl, optionally substituted C₁-C₆ alkoxy, and halogen.

36. A pharmaceutical composition comprising a therapeutically effective amount of a compound of any one of claims 1 to 35 and a pharmaceutically acceptable excipient.

37. A method of inhibiting a kinase enzyme comprising administering the compound of any one of claims 1 to 35 or the composition of claim 36 to a subject in need of treatment.

38. The method of 37, wherein the kinase is selected from the group consisting of: CLK1, CLK2, CLK3, CLK4, FMS, JNK1, JNK2, JNK3, PLK4, FLT3, FLT3 (D835V), FLT3 (ITD), FLT3 (F₆₉₁L), FLT3 (N₈₄₁I), FLT3 (D835H), FLT3 (D835Y), FLT3 (K663Q), FLT3 (N₈₄₁₁), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, LTK, abl, Akt, Aurora-A, Auroa-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, Zap70, or combinations thereof.

39. The method of claim 38, wherein the kinase is selected from the group consisting of: CLK1, CLK2, CLK3, CLK4, FMS, JNK1, JNK2, JNK3, PLK4, FLT3, FLT3 (D835V), FLT3 (ITD), FLT3 (F₆₉₁L), FLT3 (N₈₄₁I), FLT3 (D835H), FLT3 (D835Y), FLT3 (K663Q), FLT3 (N₈₄₁₁), MYLK4, NUAK2, CSFIR, DAPK3, RIOK2, HIPK1, ALK, MYLK, EGFR, FGFR1, FGFR2, FGFR3, FGFR4, FGFR5, VEGFR, JAK1, ABL1, DAPK2, and LTK.

40. The method of claim 38, wherein the kinase is selected from the group consisting of: Akt, Aurora-A, Aurora-B, Aurora-C, ATK, bcr-abl, Blk, Brk, Btk, c-Kit, c-Met, s-Src, c-fms, CDK1, CDK2 CDK4, CDK6, CDK7, CDK8, CDK9, CDK10, rRaf1, CSFIR, CSK, EGFR, ErbB2, ErbB3, ErbB4, ERK, Fak, fes, Fgr, fit-1, FLK-4, Fps, Fyn, Hck, HER, Hck, IGF-1R, INS-R, Jak, KDR, Lck, Lyn, MEK, p38, PDGFR, PIK, PKC, PYK2, Ros, Tie1, Tie2, Trk, Yes, and Zap70.

41. The method of claim 38, wherein the kinase is selected from the group consisting of CLK1, CLK4, PLK4, FLT3, and JNK1.

42. A method of treating a kinase-related disease comprising administering the compound of any one of claims 1 to 35 or the composition of claim 36 to a subject in need of treatment.

43. The method of claim 42, wherein the kinase-related disease is selected from cancer, autoimmune disease, and Duchenne muscular dystrophy.

44. A method of synthesizing the compound of any one of claims 1 to 35, a method of preparing a compound of Formula (II): 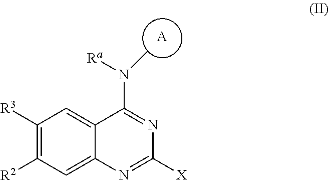
comprising mixing a compound of Formula (IIp): 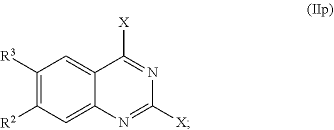
with a compound of having the following structure: 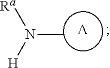
wherein:
X is a halogen atom;
R² is-OR⁶ or optionally substituted (heterocyclyl)alkynyl;
R⁶ is selected from the group consisting of methyl, optionally substituted 2-10 membered heteroalkyl, and (heterocyclyl)alkyl; and
R³ is selected from the group consisting of hydrogen, halogen, and —OMe;
R^{a} is hydrogen or optionally substituted C₁-C₁₀ alkyl; and
the A ring is an optionally substituted heteroaryl.

45. A method of synthesizing the compound of any one of claims 1 to 35, a method of preparing a compound of Formula (I): 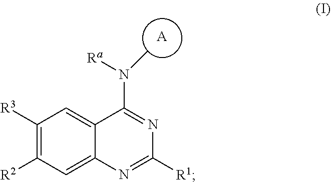
comprising mixing a compound of represented by H—R¹ with a compound of Formula (II) 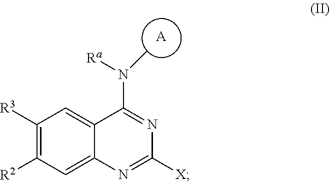
wherein:
X is a halogen atom;
R¹ is selected from the group consisting of optionally substituted 6-10 membered aryl, optionally substituted 3-10 membered heterocyclyl, optionally substituted 5-10 membered heteroaryl, optionally substituted carbamide, —CN, and —NR⁴R⁵;
each of R⁴ and R⁵ is independently selected from hydrogen, optionally substituted C₁₋₆ alkyl, or optionally substituted C₃₋₆ carbocyclyl; or alternatively, R⁴ and R⁵ taken together form an optionally substituted 3-10 membered heterocyclyl;
R² is-OR⁶ or optionally substituted (heterocyclyl)alkynyl;
R⁶ is selected from the group consisting of methyl, optionally substituted 2-10 membered heteroalkyl, and (heterocyclyl)alkyl; and
R³ is selected from the group consisting of hydrogen, halogen, and —OMe;
R^{a} is hydrogen or optionally substituted C₁-C₁₀ alkyl; and
the A ring is an optionally substituted heteroaryl.
